(19) United States
(12) Patent Application Publication
(10) Pub. No.: US 2020/0270622 A1
(43) Pub. Date: Aug. 27, 2020

(54) TISSUE-PREFERRED PROMOTERS AND METHODS OF USE

(71) Applicant: PIONEER HI-BRED INTERNATIONAL, INC., JOHNSTON, IA (US)

(72) Inventors: ALEXANDRE DA SILVA CONCEICAO, WILMINGTON, DE (US); ALLISON M FINCH, WILMINGTON, DE (US); WILLIAM JAMES GORDON-KAMM, URBANDALE, IA (US); THEODORE M KLEIN, WILMINGTON, DE (US); KEITH S LOWE, JOHNSTON, IA (US)

(73) Assignee: PIONEER HI-BRED INTERNATIONAL, INC., JOHNSTON, IA (US)

(21) Appl. No.: 16/650,334

(22) PCT Filed: Sep. 19, 2018
(86) PCT No.: PCT/US18/51697

§ 371 Date: Mar. 24, 2020

Related U.S. Application Data
(60) Provisional application No. 62/562,663, filed on Sep. 25, 2017.

(51) Int. Cl. C12N 15/82

(52) U.S. Cl. CPC C12N 15/8234; C12N 15/8241

(57) ABSTRACT

Compositions and methods for regulating expression of heterologous nucleotide sequences in a plant are provided. Compositions include tissue-preferred promoters operably linked to morphogenic genes. Also provided is a method for expressing a heterologous nucleotide sequence in a plant using a promoter sequence disclosed herein operably linked to a morphogenic gene. DNA constructs comprising a promoter operably linked to a morphogenic gene and further comprising a heterologous nucleotide sequence of interest are also provided.

### CROSS-REFERENCE TO RELATED APPLICATIONS

This application claims the benefit of PCT Application Serial Number PCT/US2018/051697, filed Sep. 19, 2018, which claims the benefit of U.S. Provisional Application No. 62/562,663, filed Sep. 25, 2017, both of which are hereby incorporated herein by reference in their entireties.

### FIELD OF THE DISCLOSURE

The present disclosure relates to the field of plant molecular biology, more particularly to regulation of gene expression in plants.

### REFERENCE TO SEQUENCE LISTING SUBMITTED ELECTRONICALLY

The official copy of the sequence listing is submitted electronically via EFS-Web as an ASCII formatted sequence listing with a file named 20200324_7453-US-PCT_ST25 created on Mar. 24, 2020 and having a size of 995,640 bytes and is filed concurrently with the specification. The sequence listing contained in this ASCII formatted document is part of the specification and is herein incorporated by reference in its entirety.

### BACKGROUND OF THE DISCLOSURE

Expression of heterologous DNA sequences in a plant host is dependent upon the presence of operably linked promoters, including promoters, that are functional within the plant host. Choice of the promoter sequence will determine when and where within the organism the heterologous DNA sequence is expressed. Where expression in specific tissues or organs is desired, tissue-preferred promoters may be used. Where gene expression in response to a stimulus is desired, inducible promoters are the regulatory element of choice. In contrast, where continuous expression is desired throughout the cells of a plant, constitutive promoters are utilized. Additional regulatory sequences upstream and/or downstream from the core promoter sequence may be included in expression constructs of transformation vectors to bring about varying levels of expression of heterologous nucleotide sequences in a transgenic plant.

Frequently it is desirable to express a DNA sequence in particular tissues or organs of a plant. For example, use of tissue-preferred promoters operably linked to morphogenic genes that promote cell proliferation are useful for the efficient recovery of transgenic events during the transformation process. Such tissue-preferred promoters also have utility in expressing trait genes and/or pathogen-resistance proteins in the desired plant tissue to enhance plant yield and resistance to pathogens. Alternatively, it might be desirable to inhibit expression of a native DNA sequence within a plant's tissues to achieve a desired phenotype. In this case, such inhibition might be accomplished with transformation of the plant to comprise a tissue-preferred promoter operably linked to an antisense nucleotide sequence, such that expression of the antisense sequence produces an RNA transcript that interferes with translation of the mRNA of the native DNA sequence.

Additionally, it may be desirable to express a DNA sequence in plant tissues that are in a particular growth or developmental phase such as, for example, cell division or elongation. Such a DNA sequence may be used to promote or inhibit plant growth processes, thereby affecting the growth rate or architecture of the plant.

Thus, there is a need for the isolation and characterization of tissue-preferred promoters, particularly promoters that can serve as regulatory elements for the controlled expression of growth stimulating genes, including morphogenic genes, that provide a strong burst of expression of the genes to stimulate in vitro growth and morphogenesis immediately after Agrobacterium-mediated transformation, which then diminishes and is virtually “off” in the tissues of a plant where ectopic overexpression would cause abnormal growth and development.

### SUMMARY OF THE DISCLOSURE

Compositions and methods for regulating gene expression in a plant are provided. More particularly, the promoters of the disclosure confer tissue-preferred expression in the epidermis L1 (outer layers) of plant tissue. Certain aspects of the disclosure include a nucleic acid molecule comprising a morphogenic gene cassette comprising a tissue-preferred promoter having a nucleotide sequence selected from the group consisting of a nucleotide sequence of at least one of SEQ ID NOS: 1-59, 108-110, 124-126, 149-152, and 189, wherein the nucleotide sequence initiates transcription in a plant cell; a nucleotide sequence having at least 95% identical to at least one of SEQ ID NOS: 1-59, 108-110, 124-126, 149-152, and 189, wherein the nucleotide sequence initiates transcription in a plant cell; a nucleotide sequence having at least 70% identity to at least one of SEQ ID NOS: 1-59, 108-110, 124-126, 149-152, and 189, wherein the nucleotide sequence initiates transcription in a plant cell; a fragment or variant of the nucleotide sequence of at least one of SEQ ID NOS: 1-59, 108-110, 124-126, 149-152, and 189, wherein the fragment or variant of the nucleotide sequence initiates transcription in a plant cell; and at least 100 contiguous nucleotides of a nucleotide sequence of at least one of SEQ ID NOS: 1-59, 108-110, 124-126, 149-152, and 189, wherein the at least 100 contiguous nucleotides of the nucleotide sequence initiates transcription in a plant cell; wherein the tissue-preferred promoter is operably linked to a morphogenic gene. Also included are expression cassettes comprising a morphogenic gene cassette. Vectors comprising an expression cassette comprising a morphogenic gene cassette are also included.

Further, plant cells and plants comprising an expression cassette comprising a morphogenic gene cassette are provided comprising a tissue-preferred promoter having a nucleotide sequence selected from the group consisting of a nucleotide sequence of at least one of SEQ ID NOS: 1-59, 108-110, 124-126, 149-152, and 189, wherein the nucleotide sequence initiates transcription in a plant cell; a nucleotide sequence having at least 95% identical to at least one of SEQ ID NOS: 1-59, 108-110, 124-126, 149-152, and 189, wherein the nucleotide sequence initiates transcription in a plant cell; a nucleotide sequence having at least 70% identity to at least one of SEQ ID NOS: 1-59, 108-110, 124-126, 149-152, and 189, wherein the nucleotide sequence initiates transcription in a plant cell; a fragment or variant of the nucleotide sequence of at least one of SEQ ID NOS: 1-59, 108-110, 124-126, 149-152, and 189, wherein the fragment or variant of the nucleotide sequence initiates transcription in a plant cell; and at least 100 contiguous nucleotides of a nucleotide sequence of at least one of SEQ ID NOS: 1-59, 108-110, 124-126, 149-152, and 189, wherein the at least 100 contiguous nucleotides of the nucleotide sequence initiates transcription in a plant cell; wherein the tissue-preferred promoter is operably linked to a morphogenic gene. Plant cells and plants comprising an expression cassette comprising a morphogenic gene cassette include monocots, dicots, and gymnosperms. Plant cells and plants comprising an expression cassette comprising a morphogenic gene cassette include monocots, dicots, and gymnosperms including, but not limited to, maize, alfalfa, sorghum, rice, millet, soybean, wheat, cotton, sunflower, barley, oats, rye, flax, sugarcane, banana, cassava, common bean, cowpea, tomato, potato, beet, grape, Eucalyptus, poplar, pine, douglas fir, citrus, papaya, cacao, cucumber, apple, Capsicum, bamboo, Triticale, melon, and Brassica are also provided. The present disclosure also provides a plant cell or plant comprising an expression cassette comprising a morphogenic gene cassette, wherein the morphogenic gene of the morphogenic gene cassette encodes a WUS/WOX homeobox polypeptide, wherein the WUS/WOX homeobox polypeptide comprises an amino acid sequence of any of SEQ ID NOS: 61, 63, 65, 67, 69, 71, 73, 75, 77, 79, 81, 83, 85, 87, 89, 91, 93, 95, 97, 99, 101, 103, 105, 107, 128, 130, 132, 134, 136, 138, 140, 142, 144, 146, or 148; or wherein the WUS/WOX homeobox polypeptide is encoded by a nucleotide sequence of any of SEQ ID NOS: 60, 62, 64, 66, 68, 70, 72, 74, 76, 78, 80, 82, 84, 86, 88, 90, 92, 94, 96, 98, 100, 102, 104, 106, 127, 129, 131, 133, 135, 137, 139, 141, 143, 145, or 147. Also provided is a plant cell or plant comprising an expression cassette comprising a morphogenic gene cassette, wherein the morphogenic gene of the morphogenic gene cassette encodes a gene product involved in plant metabolism, organ development, stem cell development, cell growth stimulation, organogenesis, regeneration, somatic embryogenesis initiation, accelerated somatic embryo maturation, initiation and/or development of the apical meristem, initiation and/or development of shoot meristem, initiation and/or development of shoots, or a combination thereof.

The present disclosure also provides plant cells and plants comprising an expression cassette comprising a morphogenic gene cassette, wherein the expression cassette further comprises a trait gene cassette comprising a heterologous polynucleotide encoding a gene product conferring nutritional enhancement, increased yield, abiotic stress tolerance, drought tolerance, cold tolerance, herbicide tolerance, pest resistance, pathogen resistance, insect resistance, nitrogen use efficiency (NUE), disease resistance, or an ability to alter a metabolic pathway. The present disclosure also provides plant cells and plants comprising an expression cassette comprising a morphogenic gene cassette, wherein the expression cassette further comprises a a trait gene cassette comprising heterologous polynucleotide encoding a gene product conferring nutritional enhancement, increased yield, abiotic stress tolerance, drought tolerance, cold tolerance, herbicide tolerance, pest resistance, pathogen resistance, insect resistance, nitrogen use efficiency (NUE), disease resistance, or an ability to alter a metabolic pathway and a site specific recombinase cassette comprising a nucleotide sequence encoding a site-specific recombinase selected from the group consisting of FLP, FLPe, KD, Cre, SSV1, lambda Int, phi C31 Int, HK022, R, B2, B3, Gin, Tn1721, CinH, ParA, Tn5053, Bxb1, TP907-1, or U153, wherein the site-specific recombinase is operably linked to a constitutive promoter, an inducible promoter, a tissue-specific promoter, or a developmentally regulated promoter. Also provides are constitutive promoters, inducible promoters, tissue-specific promoters, and developmentally regulated promoters selected from the group consisting of UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, ZM-ADF PRO (ALT2), AXIG1, DR5, XVE, GLB1, OLE, LTP2, HSP17.7, HSP26, HSP18A, AT-HSP811, AT-HSP811L, GM-HSP173B, promoters activated by tetracycline, ethamethsulfuron or chlorsulfuron, PLTP, PLTP1, PLTP2, PLTP3, SDR, LGL, LEA-14A, or LEA-D34. Also provided are plant cells and plants wherein the morphogenic gene cassette and the site-specific recombinase cassette of the expression cassette are transiently expressed in the plant cells and plants and the trait gene cassette of the expression cassette is stably incorporated into the genome of the plant cells and plants. Also provided are plant cells and plants wherein the morphogenic gene cassette and the site-specific recombinase cassette of the expression cassette are excised from the plant cells and plants and the trait gene cassette of the expression cassette is stably incorporated into the genome of the plant cells and plants. A seed of a plant is also provided wherein the seed comprises the trait gene cassette of the espression cassette.

The disclosure further provides an expression cassette comprising a recombinant polynucleotide comprising a nucleotide sequence capable of initiating transcription in a plant or a plant cell, wherein the nucleotide sequence has at least 100 contiguous nucleotides of a nucleotide sequence selected from the group consisting of at least one of SEQ ID NOS: 1-59, 108-110, 124-126, 149-152, and 189, wherein the nucleotide sequence capable of initiating transcription is operably linked to a morphogenic gene.

Also provided is an expression cassette comprising a recombinant polynucleotide comprising a functional fragment or variant capable of initiating transcription in a plant or a plant cell, wherein the functional fragment or variant is derived from a nucleotide sequence selected from the group consisting of at least one of SEQ ID NOS: 1-59, 108-110, 124-126, 149-152, and 189, wherein the functional fragment or variant capable of initiating transcription is operably linked to a morphogenic gene.

The disclosure also provides an expression cassette comprising a recombinant polynucleotide comprising a nucleotide sequence capable of initiating transcription in a plant or a plant cell, wherein the nucleotide sequence has at least 70% identity to a nucleotide sequence selected from the group consisting of at least one of SEQ ID NOS: 1-59, 108-110, 124-126, 149-152, and 189, wherein the nucleotide sequence capable of initiating transcription is operably linked to a morphogenic gene.

Also provided is an expression cassette comprising a recombinant polynucleotide comprising a nucleotide sequence capable of initiating transcription in a plant or a plant cell, wherein the nucleotide sequence has at least 95% identity to a nucleotide sequence selected from the group consisting of at least one of SEQ ID NOS: 1-59, 108-110, 124-126, 149-152, and 189, wherein the nucleotide sequence capable of initiating transcription is operably linked to a morphogenic gene.

An expression cassette comprising a recombinant polynucleotide comprising a nucleotide sequence capable of initiating transcription in a plant or a plant cell, wherein the nucleotide sequence is selected from the group consisting of at least one of SEQ ID NOS: 1-59, 108-110, 124-126, 149-152, and 189, wherein the nucleotide sequence capable of initiating transcription is operably linked to a morphogenic gene is also provided.

Also provided are methods for producing a transgenic plant, the method comprising transforming a plant cell with a recombinant expression cassette comprising (a) a tissue-preferred promoter cassette, wherein the tissue-preferred promoter cassette comprises a nucleotide sequence selected from the group consisting of: at least one of SEQ ID NOS: 1-59, 108-110, 124-126, 149-152, and 189, wherein the nucleotide sequence initiates transcription in the plant cell; a nucleotide sequence that is at least 95% identical to at least one of SEQ ID NOS: 1-59, 108-110, 124-126, 149-152, and 189, wherein the nucleotide sequence initiates transcription in the plant cell; a nucleotide sequence that is at least 70% identical to at least one of SEQ ID NOS: 1-59, 108-110, 124-126, 149-152, and 189, wherein the nucleotide sequence initiates transcription in the plant cell; a nucleotide sequence that is a fragment or variant of at least one of SEQ ID NOS: 1-59, 108-110, 124-126, 149-152, and 189, wherein the nucleotide sequence initiates transcription in the plant cell; or a nucleotide sequence that is at least a 100-bp fragment of at least one of SEQ ID NOS: 1-59, 108-110, 124-126, 149-152, and 189, wherein the nucleotide sequence initiates transcription in the plant cell, wherein the nucleotide sequence is operably linked to a morphogenic gene and (b) a trait gene cassette comprising a heterologous polynucleotide of interest encoding a gene product conferring nutritional enhancement, increased yield, abiotic stress tolerance, drought tolerance, cold tolerance, herbicide tolerance, pest resistance, pathogen resistance, insect resistance, nitrogen use efficiency (NUE), disease resistance, or an ability to alter a metabolic pathway; selecting a transgenic plant cell having the recombinant expression cassette; and regenerating the transgenic plant from the transgenic plant cell.

Also provided are monocot plant cells, dicot plant cells, and gymnosperm plant cells useful in the methods for producing a transgenic plant of the disclosure. Plant cells useful in the methods of the disclosure include monocots, dicots, and gymnosperms including, but not limited to, maize, alfalfa, sorghum, rice, millet, soybean, wheat, cotton, sunflower, barley, oats, rye, flax, sugarcane, banana, cassava, common bean, cowpea, tomato, potato, beet, grape, Eucalyptus, poplar, pine, douglas fir, citrus, papaya, cacao, cucumber, apple, Capsicum, bamboo, Triticale, melon, and Brassica are also provided.

The present disclosure also provides methods for producing a transgenic plant, the method comprising transforming a plant cell with a recombinant expression cassette comprising a tissue-preferred promoter cassette disclosed herein, wherein the morphogenic gene encodes a WUS/WOX homeobox polypeptide, wherein the WUS/WOX homeobox polypeptide comprises an amino acid sequence of any of SEQ ID NOS: 61, 63, 65, 67, 69, 71, 73, 75, 77, 79, 81, 83, 85, 87, 89, 91, 93, 95, 97, 99, 101, 103, 105, 107, 128, 130, 132, 134, 136, 138, 140, 142, 144, 146, or 148; or wherein the WUS/WOX homeobox polypeptide is encoded by a nucleotide sequence of any of SEQ ID NOS: 60, 62, 64, 66, 68, 70, 72, 74, 76, 78, 80, 82, 84, 86, 88, 90, 92, 94, 96, 98, 100, 102, 104, 106, 127, 129, 131, 133, 135, 137, 139, 141, 143, 145, or 147. Also provided are methods for producing a transgenic plant, the method comprising transforming a plant cell with a recombinant expression cassette comprising a tissue-preferred promoter cassette disclosed herein, wherein the morphogenic gene encodes a gene product involved in plant metabolism, organ development, stem cell development, cell growth stimulation, organogenesis, regeneration, somatic embryogenesis initiation, accelerated somatic embryo maturation, initiation and/or development of the apical meristem, initiation and/or development of shoot meristem, initiation and/or development of shoots, or a combination thereof.

Also provided are methods for producing a transgenic plant, the method comprising transforming a plant cell with a recombinant expression cassette comprising a tissue-preferred promoter cassette disclosed herein, wherein the recombinant expression cassette further comprises a site-specific recombinase cassette comprising a nucleotide sequence encoding a site-specific recombinase selected from the group consisting of FLP, FLPe, KD, Cre, SSV1, lambda Int, phi C31 Int, HK022, R, B2, B3, Gin, Tn1721, CinH, ParA, Tn5053, Bxb1, TP907-1, or U153, wherein the site-specific recombinase is operably linked to a constitutive promoter, an inducible promoter, a tissue-specific promoter, or a developmentally regulated promoter. Also provided are methods for producing a transgenic plant, the method comprising transforming a plant cell with a recombinant expression cassette comprising a tissue-preferred promoter cassette and a site-specific recombinase cassette disclosed herein, wherein the constitutive promoter, the inducible promoter, the tissue-specific promoter, or the developmentally regulated promoter is selected from the group consisting of UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, ZM-ADF PRO (ALT2), AXIG1, DR5, XVE, GLB1, OLE, LTP2, HSP17.7, HSP26, HSP18A, AT-HSP811, AT-HSP811L, GM-HSP173B, promoters activated by tetracycline, ethamethsulfuron or chlorsulfuron, PLTP, PLTP1, PLTP2, PLTP3, SDR, LGL, LEA-14A, or LEA-D34.

Also provided are methods for producing a transgenic plant, wherein the method further comprises excising the tissue-preferred promoter cassette and the site-specific recombinase cassette from the recombinant expression cassette. Transgenic plants produced by the methods disclosed herein are also provided. Seed containing the trait gene cassette of the recombinant expression cassette produced from the transgenic plants produced by the methods disclosed herein are also provided.

Also provided are methods for producing a transgenic plant, wherein the tissue-preferred promoter cassette comprises a first T-DNA and the trait gene cassette comprises a second T-DNA. Also provided are methods for producing a transgenic plant, wherein the first T-DNA and the second T-DNA reside in the same bacterial strain for transforming the plant cell. Also provided are methods for producing a transgenic plant, wherein the methods further comprise segregating the first T-DNA away from the second T-DNA. Transgenic plants so produced are also provided. Seed of the transgenic plants so produced, wherein the seed comprises the trait gene cassette of the recombinant expression cassette are also provided.

Also provided are methods for producing a transgenic plant, wherein the first T-DNA resides in a first bacterial strain and the second T-DNA resides in a second bacterial strain and the first bacterial strain and the second bacterial strain are mixed in a ratio for transforming the plant cell. Also provided are methods for producing a transgenic plant, wherein the methods further comprise segregating the first T-DNA away from the second T-DNA. Transgenic plants so produced are also provided. Seed of the transgenic plants so produced, wherein the seed comprises the trait gene cassette of the recombinant expression cassette are also provided.

Methods of improving somatic embryo maturation efficiency, the method comprising transforming a plant cell with a recombinant expression cassette comprising (a) a tissue-preferred promoter cassette, wherein the tissue-preferred promoter cassette comprises a nucleotide sequence selected from the group consisting of: at least one of SEQ ID NOS: 1-59, 108-110, 124-126, 149-152, and 189, wherein the nucleotide sequence initiates transcription in the plant cell; a nucleotide sequence that is at least 95% identical to at least one of SEQ ID NOS: 1-59, 108-110, 124-126, 149-152, and 189, wherein the nucleotide sequence initiates transcription in the plant cell; a nucleotide sequence that is at least 70% identical to at least one of SEQ ID NOS: 1-59, 108-110, 124-126, 149-152, and 189, wherein the nucleotide sequence initiates transcription in the plant cell; a nucleotide sequence that is a fragment or variant of at least one of SEQ ID NOS: 1-59, 108-110, 124-126, 149-152, and 189, wherein the nucleotide sequence initiates transcription in the plant cell; or a nucleotide sequence that is at least a 100-bp fragment of at least one of SEQ ID NOS: 1-59, 108-110, 124-126, 149-152, and 189, wherein the nucleotide sequence initiates transcription in the plant cell, wherein the nucleotide sequence is operably linked to a morphogenic gene and (b) a trait gene cassette comprising a heterologous polynucleotide of interest encoding a gene product conferring nutritional enhancement, increased yield, abiotic stress tolerance, drought tolerance, cold tolerance, herbicide tolerance, pest resistance, pathogen resistance, insect resistance, nitrogen use efficiency (NUE), disease resistance, or an ability to alter a metabolic pathway; selecting a transgenic plant cell having the recombinant expression cassette; and regenerating the transgenic plant from the transgenic plant cell, wherein the recombinant expression cassette results in improved somatic embryo maturation efficiency when compared to a transgenic plant cell not comprising the recombinant expression cassette.

Also provided are monocot plant cells, dicot plant cells, and gymnosperm plant cells useful in the methods of improving somatic embryo maturation efficiency of the disclosure. Plant cells useful in the methods of the disclosure include monocots, dicots, and gymnosperms including, but not limited to, maize, alfalfa, sorghum, rice, millet, soybean, wheat, cotton, sunflower, barley, oats, rye, flax, sugarcane, banana, cassava, common bean, cowpea, tomato, potato, beet, grape, Eucalyptus, poplar, pine, douglas fir, citrus, papaya, cacao, cucumber, apple, Capsicum, bamboo, Triticale, melon, and Brassica are also provided. The present disclosure also provides methods of improving somatic embryo maturation efficiency, the method comprising transforming a plant cell with a recombinant expression cassette comprising a tissue-preferred promoter cassette disclosed herein, wherein the morphogenic gene encodes a WUS/WOX homeobox polypeptide, wherein the WUS/WOX homeobox polypeptide comprises an amino acid sequence of any of SEQ ID NOS: 61, 63, 65, 67, 69, 71, 73, 75, 77, 79, 81, 83, 85, 87, 89, 91, 93, 95, 97, 99, 101, 103, 105, 107, 128, 130, 132, 134, 136, 138, 140, 142, 144, 146, or 148; or wherein the WUS/WOX homeobox polypeptide is encoded by a nucleotide sequence of any of SEQ ID NOS: 60, 62, 64, 66, 68, 70, 72, 74, 76, 78, 80, 82, 84, 86, 88, 90, 92, 94, 96, 98, 100, 102, 104, 106, 127, 129, 131, 133, 135, 137, 139, 141, 143, 145, or 147. Also provided are methods of improving somatic embryo maturation efficiency, the method comprising transforming a plant cell with a recombinant expression cassette comprising a tissue-preferred promoter cassette disclosed herein, wherein the morphogenic gene encodes a gene product involved in plant metabolism, organ development, stem cell development, cell growth stimulation, organogenesis, regeneration, somatic embryogenesis initiation, accelerated somatic embryo maturation, initiation and/or development of the apical meristem, initiation and/or development of shoot meristem, initiation and/or development of shoots, or a combination thereof.

Also provided are methods of improving somatic embryo maturation efficiency, the method comprising transforming a plant cell with a recombinant expression cassette comprising a tissue-preferred promoter cassette disclosed herein, wherein the recombinant expression cassette further comprises a site-specific recombinase cassette comprising a nucleotide sequence encoding a site-specific recombinase selected from the group consisting of FLP, FLPe, KD, Cre, SSV1, lambda Int, phi C31 Int, HK022, R, B2, B3, Gin, Tn1721, CinH, ParA, Tn5053, Bxb1, TP907-1, or U153, wherein the site-specific recombinase is operably linked to a constitutive promoter, an inducible promoter, a tissue-specific promoter, or a developmentally regulated promoter. Also provided are methods of improving somatic embryo maturation efficiency, the method comprising transforming a plant cell with a recombinant expression cassette comprising a tissue-preferred promoter cassette and a site-specific recombinase cassette disclosed herein, wherein the constitutive promoter, the inducible promoter, the tissue-specific promoter, or the developmentally regulated promoter is selected from the group consisting of UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, ZM-ADF PRO (ALT2), AXIG1, DR5, XVE, GLB1, OLE, LTP2, HSP17.7, HSP26, HSP18A, AT-HSP811, AT-HSP811L, GM-HSP173B, promoters activated by tetracycline, ethamethsulfuron or chlorsulfuron, PLTP, PLTP1, PLTP2, PLTP3, SDR, LGL, LEA-14A, or LEA-D34.

Also provided are methods of improving somatic embryo maturation efficiency, wherein the method further comprises excising the tissue-preferred promoter cassette and the site-specific recombinase cassette from the recombinant expression cassette. Transgenic plants produced by the methods disclosed herein are also provided. Seed containing the trait gene cassette of the recombinant expression cassette produced from the transgenic plants produced by the methods disclosed herein are also provided.

Also provided are methods of improving somatic embryo maturation efficiency, wherein the tissue-preferred promoter cassette comprises a first T-DNA and the trait gene cassette comprises a second T-DNA. Also provided are methods of improving somatic embryo maturation efficiency, wherein the first T-DNA and the second T-DNA reside in the same bacterial strain for transforming the plant cell. Also provided are methods of improving somatic embryo maturation efficiency, wherein the methods further comprise segregating the first T-DNA away from the second T-DNA. Transgenic plants so produced are also provided. Seed of the transgenic plants so produced, wherein the seed comprises the trait gene cassette of the recombinant expression cassette are also provided.

Also provided are methods of improving somatic embryo maturation efficiency, wherein the first T-DNA resides in a first bacterial strain and the second T-DNA resides in a second bacterial strain and the first bacterial strain and the second bacterial strain are mixed in a ratio for transforming the plant cell. Also provided are methods of improving somatic embryo maturation efficiency, wherein the methods further comprise segregating the first T-DNA away from the second T-DNA. Transgenic plants so produced are also provided. Seed of the transgenic plants so produced, wherein the seed comprises the trait gene cassette of the recombinant expression cassette are also provided.

Also provided are methods for producing a transgenic dicot plant or a transgenic gymnosperm plant, the method comprising transforming a dicot plant cell or a gymnosperm plant cell with a recombinant expression cassette comprising (a) a tissue-preferred promoter cassette, wherein the tissue-preferred promoter cassette comprises a nucleotide sequence selected from the group consisting of: at least one of SEQ ID NOS: 1-59, 108-110, 124-126, 149-152, and 189, wherein the nucleotide sequence initiates transcription in the plant cell; a nucleotide sequence that is at least 95% identical to at least one of SEQ ID NOS: 1-59, 108-110, 124-126, 149-152, and 189, wherein the nucleotide sequence initiates transcription in the plant cell; a nucleotide sequence that is at least 70% identical to at least one of SEQ ID NOS: 1-59, 108-110, 124-126, 149-152, and 189, wherein the nucleotide sequence initiates transcription in the plant cell; a nucleotide sequence that is a fragment or variant of at least one of SEQ ID NOS: 1-59, 108-110, 124-126, 149-152, and 189, wherein the nucleotide sequence initiates transcription in the plant cell; or a nucleotide sequence that is at least a 100-bp fragment of at least one of SEQ ID NOS: 1-59, 108-110, 124-126, 149-152, and 189, wherein the nucleotide sequence initiates transcription in the plant cell, wherein the nucleotide sequence initiating transcription in the plant cell is operably linked to a nucleotide sequence encoding a WUS/WOX homeobox polypeptide and (b) a trait gene cassette comprising a heterologous polynucleotide of interest encoding a gene product conferring nutritional enhancement, increased yield, abiotic stress tolerance, drought tolerance, cold tolerance, herbicide tolerance, pest resistance, pathogen resistance, insect resistance, nitrogen use efficiency (NUE), disease resistance, or an ability to alter a metabolic pathway; allowing expression of the recombinant expression cassette in each transformed plant cell to form a somatic embryo or a shoot; and germinating the somatic embryo or culturing the shoot to form the transgenic dicot plant or the transgenic gymnosperm plant wherein the transgenic dicot plant or the transgenic gymnosperm plant comprises the heterologous polynucleotide of interest. The disclosed method also provides the somatic embryo or the shoot formation within about 21 to about 28 days after initiation of transforming the dicot cell or the gymnosperm cell.

Also provided are gymnosperm plant cells useful in the methods for producing a transgenic plant of the disclosure. Gymnosperm plant cells useful in the methods of the disclosure including, but not limited to, pine and douglas fir are also provided. Also provided are dicot plant cells useful in the methods for producing a transgenic plant of the disclosure. Dicot plant cells useful in the methods of the disclosure including, but not limited to, alfalfa, soybean, cotton, sunflower, flax, cassava, common bean, cowpea, tomato, potato, beet, grape, Eucalyptus, poplar, citrus, papaya, cacao, cucumber, apple, Capsicum, melon, or Brassica are also provided.

The present disclosure also provides methods for producing a transgenic dicot plant or a transgenic gymnosperm plant, the method comprising transforming a dicot plant cell or a gymnosperm plant cell with a recombinant expression cassette comprising a tissue-preferred promoter cassette disclosed herein, wherein the WUS/WOX homeobox polypeptide comprises an amino acid sequence of any of SEQ ID NOS: 61, 63, 65, 67, 69, 71, 73, 75, 77, 79, 81, 83, 85, 87, 89, 91, 93, 95, 97, 99, 101, 103, 105, 107, 128, 130, 132, 134, 136, 138, 140, 142, 144, 146, or 148; or wherein the WUS/WOX homeobox polypeptide is encoded by a nucleotide sequence of any of SEQ ID NOS: 60, 62, 64, 66, 68, 70, 72, 74, 76, 78, 80, 82, 84, 86, 88, 90, 92, 94, 96, 98, 100, 102, 104, 106, 127, 129, 131, 133, 135, 137, 139, 141, 143, 145, or 147. Also provided are methods for producing a transgenic plant, the method comprising transforming a plant cell with a recombinant expression cassette comprising a tissue-preferred promoter cassette disclosed herein, wherein the nucleotide sequence encoding the WUS/WOX homeobox polypeptide encodes a gene product involved in plant metabolism, organ development, stem cell development, cell growth stimulation, organogenesis, regeneration, somatic embryogenesis initiation, accelerated somatic embryo maturation, initiation and/or development of the apical meristem, initiation and/or development of shoot meristem, initiation and/or development of shoots, or a combination thereof.

Also provided are methods for producing a a transgenic dicot plant or a transgenic gymnosperm plant, the method comprising transforming a dicot plant cell or a gymnosperm plant cell with a recombinant expression cassette comprising a tissue-preferred promoter cassette disclosed herein, wherein the recombinant expression cassette further comprises a site-specific recombinase cassette comprising a nucleotide sequence encoding a site-specific recombinase selected from the group consisting of FLP, FLPe, KD, Cre, SSV1, lambda Int, phi C31 Int, HK022, R, B2, B3, Gin, Tn1721, CinH, ParA, Tn5053, Bxb1, TP907-1, or U153, wherein the site-specific recombinase is operably linked to a constitutive promoter, an inducible promoter, a tissue-specific promoter, or a developmentally regulated promoter. Also provided are methods for producing a transgenic dicot plant or a transgenic gymnosperm plant, the method comprising transforming a dicot plant cell or a gymnosperm plant cell with a recombinant expression cassette comprising a tissue-preferred promoter cassette and a site-specific recombinase cassette disclosed herein, wherein the constitutive promoter, the inducible promoter, the tissue-specific promoter, or the developmentally regulated promoter is selected from the group consisting of UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, ZM-ADF PRO (ALT2), AXIG1, DR5, XVE, GLB1, OLE, LTP2, HSP17.7, HSP26, HSP18A, AT-HSP811, AT-HSP811L, GM-HSP173B, promoters activated by tetracycline, ethamethsulfuron or chlorsulfuron, PLTP, PLTP1, PLTP2, PLTP3, SDR, LGL, LEA-14A, or LEA-D34.

Also provided are methods for producing a transgenic dicot plant or a transgenic gymnosperm plant, wherein the method further comprises excising the tissue-preferred promoter cassette and the site-specific recombinase cassette from the recombinant expression cassette. Transgenic dicot plants or transgenic gymnosperm plantsproduced by the methods disclosed herein are also provided. Seed containing the trait gene cassette of the recombinant expression cassette produced from the transgenic dicot plants or the transgenic gymnosperm plants produced by the methods disclosed herein are also provided.

Also provided are methods for producing transgenic dicot plants or transgenic gymnosperm plants, wherein the tissue-preferred promoter cassette comprises a first T-DNA and the trait gene cassette comprises a second T-DNA. Also provided are methods for producing a transgenic dicot plant or a transgenic gymnosperm plant, wherein the first T-DNA and the second T-DNA reside in the same bacterial strain for transforming the plant cell. Also provided are methods for producing a transgenic dicot plant or a transgenic gymnosperm plant, wherein the methods further comprise segregating the first T-DNA away from the second T-DNA. Transgenic plants so produced are also provided. Seed of the transgenic dicot plants or transgenic gymnosperm plants so produced, wherein the seed comprises the trait gene cassette of the recombinant expression cassette are also provided.

Also provided are methods for producing a transgenic dicot plant or a transgenic gymnosperm plant, wherein the first T-DNA resides in a first bacterial strain and the second T-DNA resides in a second bacterial strain and the first bacterial strain and the second bacterial strain are mixed in a ratio for transforming the dicot plant cell or the gymnosperm plant cell. Also provided are methods for producing a transgenic dicot plant or a transgenic gymnosperm plant, wherein the methods further comprise segregating the first T-DNA away from the second T-DNA. Transgenic plants so produced are also provided. Seed of the transgenic dicot plants or transgenic gymnosperm plants so produced, wherein the seed comprises the trait gene cassette of the recombinant expression cassette are also provided.

Also provided are methods for producing a transgenic dicot plant or a transgenic gymnosperm plant, comprising: (a) transforming a cell of a dicot explant or a gymnosperm explant with a recombinant expression cassette comprising a trait gene cassette comprising a heterologous gene of interest and a morphogenic gene cassette comprising a nucleotide sequence encoding a WUS/WOX homeobox polypeptide; (b) allowing expression of the recombinant expression cassette of (a) in each transformed cell to form a somatic embryo or a shoot; and (c) germinating the somatic embryo or culturing the shoot to form the transgenic dicot plant or the transgenic gymnosperm plant. The disclosed method also provides the somatic embryo or the shoot formation within about 21 to about 28 days after initiation of transforming the dicot cell or the gymnosperm cell.

Also provided are dicot plant cells and gymnosperm plant cells useful in the methods for producing a transgenic dicot plant or a transgenic gymnosperm plant of the disclosure. Plant cells useful in the methods of the disclosure include dicots and gymnosperms including, but not limited to, alfalfa, soybean, cotton, sunflower, flax, cassava, common bean, cowpea, tomato, potato, beet, grape, Eucalyptus, poplar, pine, douglas fir, citrus, papaya, cacao, cucumber, apple, Capsicum, melon, or Brassica.

The present disclosure also provides methods for producing a transgenic dicot plant or a transgenic gymnosperm plant, wherein the WUS/WOX homeobox polypeptide comprises an amino acid sequence of any of SEQ ID NOS: 61, 63, 65, 67, 69, 71, 73, 75, 77, 79, 81, 83, 85, 87, 89, 91, 93, 95, 97, 99, 101, 103, 105, 107, 128, 130, 132, 134, 136, 138, 140, 142, 144, 146, or 148; or wherein the WUS/WOX homeobox polypeptide is encoded by a nucleotide sequence of any of SEQ ID NOS: 60, 62, 64, 66, 68, 70, 72, 74, 76, 78, 80, 82, 84, 86, 88, 90, 92, 94, 96, 98, 100, 102, 104, 106, 127, 129, 131, 133, 135, 137, 139, 141, 143, 145, or 147. Also provided are methods for producing a transgenic dicot plant or a transgenic gymnosperm plant, wherein the WUS/WOX homeobox polypeptide is encoded by a nucleotide that encodes a gene product involved in plant metabolism, organ development, stem cell development, cell growth stimulation, organogenesis, regeneration, somatic embryogenesis initiation, accelerated somatic embryo maturation, initiation and/or development of the apical meristem, initiation and/or development of shoot meristem, initiation and/or development of shoots, or a combination thereof.

Also provided are methods for producing a transgenic dicot plant or a transgenic gymnosperm plant, wherein the the WUS/WOX homeobox polypeptide is operably linked to a promoter selected from the group consisting of a constitutive promoter, an inducible promoter, a tissue-specific promoter, or a developmentally regulated promoter. Also provided are methods for producing a transgenic dicot plant or a transgenic gymnosperm plant, wherein the constitutive promoter, the inducible promoter, the tissue-specific promoter, or the developmentally regulated promoter is selected from the group consisting of UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, ZM-ADF PRO (ALT2), AXIG1, DR5, XVE, GLB1, OLE, LTP2, HSP17.7, HSP26, HSP18A, AT-HSP811, AT-HSP811L, GM-HSP173B, promoters activated by tetracycline, ethamethsulfuron or chlorsulfuron, PLTP, PLTP1, PLTP2, PLTP3, SDR, LGL, LEA-14A, LEA-D34, at least one of SEQ ID NOS: 1-59, 108-110, 124-126, 149-152, and 189, a nucleotide sequence that is at least 95% identical to at least one of SEQ ID NOS: 1-59, 108-110, 124-126, 149-152, and 189, a nucleotide sequence that is at least 70% identical to at least one of SEQ ID NOS: 1-59, 108-110, 124-126, 149-152, and 189, a fragment or variant of at least one of SEQ ID NOS: 1-59, 108-110, 124-126, 149-152, and 189, or at least a 100-bp fragment of at least one of SEQ ID NOS: 1-59, 108-110, 124-126, 149-152, and 189.

Also provided are methods for producing a transgenic dicot plant or a transgenic gymnosperm plant, wherein the heterologous polynucleotide of interest encodes a gene product conferring nutritional enhancement, increased yield, abiotic stress tolerance, drought tolerance, cold tolerance, herbicide tolerance, pest resistance, pathogen resistance, insect resistance, nitrogen use efficiency (NUE), disease resistance, or an ability to alter a metabolic pathway.

Also provided are methods for producing a a transgenic dicot plant or a transgenic gymnosperm plant, wherein the recombinant expression cassette further comprises a site-specific recombinase cassette comprising a nucleotide sequence encoding a site-specific recombinase selected from the group consisting of FLP, FLPe, KD, Cre, SSV1, lambda Int, phi C31 Int, HK022, R, B2, B3, Gin, Tn1721, CinH, ParA, Tn5053, Bxb1, TP907-1, or U153, wherein the site-specific recombinase is operably linked to a constitutive promoter, an inducible promoter, a tissue-specific promoter, or a developmentally regulated promoter. Also provided are methods for producing a transgenic dicot plant or a transgenic gymnosperm plant, wherein the constitutive promoter, the inducible promoter, the tissue-specific promoter, or the developmentally regulated promoter is selected from the group consisting of UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, ZM-ADF PRO (ALT2), AXIG1, DR5, XVE, GLB1, OLE, LTP2, HSP17.7, HSP26, HSP18A, AT-HSP811, AT-HSP811L, GM-HSP173B, promoters activated by tetracycline, ethamethsulfuron or chlorsulfuron, PLTP, PLTP1, PLTP2, PLTP3, SDR, LGL, LEA-14A, or LEA-D34.

Also provided are methods for producing a transgenic dicot plant or a transgenic gymnosperm plant, wherein the method further comprises excising the morphogenic gene cassett and the site-specific recombinase cassette from the recombinant expression cassette. Transgenic dicot plant or transgenic gymnosperm plant produced by the methods disclosed herein are also provided. Seed containing the trait gene cassette of the recombinant expression cassette produced from the transgenic dicot plants or the transgenic gymnosperm plants produced by the methods disclosed herein are also provided.

Also provided are methods for producing a transgenic dicot plant or a transgenic gymnosperm plant, wherein the morphogenic gene cassette comprises a first T-DNA and the trait gene cassette comprises a second T-DNA. Also provided are methods for producing a transgenic dicot plant or a transgenic gymnosperm plant, wherein the first T-DNA and the second T-DNA reside in the same bacterial strain for transforming the plant cell. Also provided are methods for producing a transgenic dicot plant or a transgenic gymnosperm plant, wherein the methods further comprise segregating the first T-DNA away from the second T-DNA. Transgenic dicot plants and transgenic gymnosperm plants so produced are also provided. Seed of the transgenic dicot plants and transgenic gymnosperm plants so produced, wherein the seed comprises the trait gene cassette of the recombinant expression cassette are also provided.

Also provided are methods for producing a transgenic dicot plant or a transgenic gymnosperm plant, wherein the first T-DNA resides in a first bacterial strain and the second T-DNA resides in a second bacterial strain and the first bacterial strain and the second bacterial strain are mixed in a ratio for transforming the plant cell. Also provided are methods for producing a transgenic dicot plant or a transgenic gymnosperm plant, wherein the methods further comprise segregating the first T-DNA away from the second T-DNA. Transgenic plants so produced are also provided. Seed of the transgenic dicot plants and transgenic gymnosperm plants so produced, wherein the seed comprises the trait gene cassette of the recombinant expression cassette are also provided.

Also provided are methods for producing a transgenic dicot plant or a transgenic gymnosperm plant, wherein germinating comprises transferring the somatic embryo or shoot to a maturation medium or germination medium and forming the transgenic dicot plant of the transgenic gymnosperm plant.

### DESCRIPTION OF THE FIGURES

FIG. 1 shows the median somatic embryo maturation efficiency of promoters GM-HBSTART2 (HBS2; SEQ ID NO:108), GM-HBSTART3 (HBS3; SEQ ID NO:1), GM-LTP3 (LTP3; SEQ ID NO:124), GM-MATE1 (MATE1; SEQ ID NO:109), GM-NED1 (NED1; SEQ ID NO:110) driving WUS expression (HBS2 (PHP80734; SEQ ID NO:113), HBS3 (PHP81343; SEQ ID NO:116), LTP3 (PHP80730; SEQ ID NO:112), MATE1 (PHP80736; SEQ ID NO:114) and NED1 (PHP81060; SEQ ID NO:115)) and the TAG-RFP control (no WUS gene) (PHP80728; SEQ ID NO:111).

FIG. 2A, FIG. 2B, and FIG. 2C pictures were taken under white light. FIG. 2D, FIG. 2E, and FIG. 2F pictures were taken under fluorescent light to show the transgenic events. FIG. 2A, FIG. 2B, FIG. 2C, FIG. 2D, FIG. 2E, and FIG. 2F pictures were taken at the same time (5-6 weeks after Agrobacterium-infection). The somatic embryos shown in FIG. 2A and FIG. 2D, the TAG-RFP control treatment (Control TAG-RFP; PHP80728; SEQ ID NO:111), are small and underdeveloped. The somatic embryos shown in FIG. 2B and FIG. 2E, the LTP3 PRO::WUS (PHP80730; SEQ ID NO:112) treatment are approximately 2-fold larger than those in the TAG-RFP control treatment. The somatic embryos shown in FIG. 2C and FIG. 2F, the HBS3 PRO::WUS (PHP81343; SEQ ID NO:116) treatment are approximately 5-fold larger than those in the TAG-RFP control treatment.

FIG. 3A and FIG. 3B illustrate normal development and seed set of GM-HBS3 PRO::WUS events.

### DETAILED DESCRIPTION

The disclosures herein will be described more fully hereinafter with reference to the accompanying drawings, in which some, but not all possible aspects are shown. Indeed, disclosures may be embodied in many different forms and should not be construed as limited to the aspects set forth herein; rather, these aspects are provided so that this disclosure will satisfy applicable legal requirements.

Many modifications and other aspects disclosed herein will come to mind to one skilled in the art to which the disclosed compositions and methods pertain having the benefit of the teachings presented in the following descriptions and the associated drawings. Therefore, it is to be understood that the disclosures are not to be limited to the specific aspects disclosed and that modifications and other aspects are intended to be included within the scope of the appended claims. Although specific terms are employed herein, they are used in a generic and descriptive sense only and not for purposes of limitation.

It is also to be understood that the terminology used herein is for the purpose of describing particular aspects only and is not intended to be limiting. As used in the specification and in the claims, the term “comprising” can include the aspect of “consisting of.” Unless defined otherwise, all technical and scientific terms used herein have the same meaning as commonly understood by one of ordinary skill in the art to which the disclosed compositions and methods belong. In this specification and in the claims which follow, reference will be made to a number of terms which shall be defined herein.

Regenerable plant structure is defined as a multicellular structure capable of forming a fully functional fertile plant, such as, but not limited to, shoot meristem, shoots, somatic embryos, embryogenic callus, somatic meristems, and/or organogenic callus.

Somatic embryo is defined as a multicellular structure that progresses through developmental stages that are similar to the development of a zygotic embryo, including formation of globular and transition-stage embryos, formation of an embryo axis and a scutellum, and accumulation of lipids and starch. Single somatic embryos derived from a zygotic embryo germinate to produce single non-chimeric plants, which may originally derive from a single-cell.

Embryogenic callus is defined as a friable or non-friable mixture of undifferentiated or partially undifferentiated cells which subtend proliferating primary and secondary somatic embryos capable of regenerating into mature fertile plants.

Somatic meristem is defined as a multicellular structure that is similar to the apical meristem which is part of a seed-derived embryo, characterized as having an undifferentiated apical dome flanked by leaf primorida and subtended by vascular initials, the apical dome giving rise to an above-ground vegetative plant. Such somatic meristems can form single or fused clusters of meristems.

Organogenic callus is defined as a compact mixture of differentiated growing plant structures, including but not limited to apical meristems, root meristems, leaves and roots.

Germination is the growth of a regenerable structure to form a plantlet which continues growing to produce a plant.

A transgenic plant is defined as a mature, fertile plant that contains a transgene.

The disclosure relates to compositions and methods drawn to a nucleic acid molecule comprising a tissue-preferred promoter operably linked to a morphogenic gene and methods of their use. The nucleic acid molecule compositions of the disclosure comprise nucleotide sequences for tissue-preferred promoters known as GM-HBSTART3 (SEQ ID NO: 1), GM-HBSTART3 (TRUNCATED) (SEQ ID NO: 2), AT-ML1 (SEQ ID NO: 3), GM-ML1-Like (SEQ ID NO: 4), GM-ML1-Like (TRUNCATED) (SEQ ID NO: 5), ZM-HBSTART3 (SEQ ID NO: 6), OS-HBSTART3 (SEQ ID NO: 7), AT-PDF1 P2 (SEQ ID NO: 8), GM-PDF1 (SEQ ID NO: 9), GM-PDF1 (TRUNCATED) (SEQ ID NO: 10), SB-PDF1 (SEQ ID NO: 11), OS-PDF1 (SEQ ID NO: 12), OS-PDF1 (TRUNCATED) (SEQ ID NO: 13), PT-PDF1 (SEQ ID NO: 14), PT-PDF1 (TRUNCATED) (SEQ ID NO: 15), SI-PDF1 (SEQ ID NO: 16), SI-PDF1 (TRUNCATED) (SEQ ID NO: 17), AT-PDF2 (SEQ ID NO: 18), GM-PDF2 (SEQ ID NO: 19), GM-PDF2 (TRUNCATED) (SEQ ID NO: 20), ZM-GL1 (SEQ ID NO: 21), AT-PDF2a (SEQ ID NO: 22), AT-PDF2a (TRUNCATED) (SEQ ID NO: 23), GM-PDF2a (SEQ ID NO: 24), GM-PDF2a (TRUNCATED) (SEQ ID NO: 25), OS-PDF2 (SEQ ID NO: 26), OS-PDF2 (TRUNCATED) (SEQ ID NO: 27), PT-PDF2 (SEQ ID NO: 28), PT-PDF2 (TRUNCATED) (SEQ ID NO: 29), VV-PDF2 (SEQ ID NO: 30), VV-PDF2 (TRUNCATED) (SEQ ID NO: 31), ZM-PDF2 (SEQ ID NO: 32), SI-PDF2 (SEQ ID NO: 33), SI-PDF2 (TRUNCATED) (SEQ ID NO: 34), VV-PDF2a (SEQ ID NO: 35), PT-PDF2a (SEQ ID NO: 36), PT-PDF2a (TRUNCATED) (SEQ ID NO: 37), MT-PDF2 (SEQ ID NO: 38), MT-PDF2 (TRUNCATED) (SEQ ID NO: 39), AT-HDG2 (SEQ ID NO: 40), GM-HDG2 (SEQ ID NO: 41), GM-HDG2 (TRUNCATED) (SEQ ID NO: 42), SB-HDG2 (SEQ ID NO: 43), SB-HDG2 (TRUNCATED) (SEQ ID NO: 44), AT-CER6 (SEQ ID NO: 45), AT-CER60 (SEQ ID NO: 46), AT-CER60 (TRUNCATED) (SEQ ID NO: 47), GM-CER6 (SEQ ID NO: 48), GM-CER6 (TRUNCATED) (SEQ ID NO: 49), PT-CER6 (SEQ ID NO: 50), PT-CER6 (TRUNCATED) (SEQ ID NO: 51), VV-CER6 (SEQ ID NO: 52), VV-CER6 (TRUNCATED) (SEQ ID NO: 53), SB-CER6 (SEQ ID NO: 54), ZM-CER6 (SEQ ID NO: 55), SI-CER6 (SEQ ID NO: 56), SI-CER6 (TRUNCATED) (SEQ ID NO: 57), OS-CER6 (SEQ ID NO: 58), OS-CER6 (TRUNCATED) (SEQ ID NO: 59), GM-HBSTART2 (SEQ ID NO: 108), GM-MATE1 (SEQ ID NO: 109), GM-NED1 (SEQ ID NO: 110), GM-LTP3 (SEQ ID NO:124), AT-ML1 (TRUNCATED) (SEQ ID NO: 125), AT-CER6 (TRUNCATED1) (SEQ ID NO: 126), AT-PDF1 (TRUNCATED) (SEQ ID NO: 149), AT-PDF2 (TRUNCATED) (SEQ ID NO: 150), AT-HDG2 (TRUNCATED) (SEQ ID NO: 151), AT-ANL2 (SEQ ID NO: 152), and AT-CER6 (TRUNCATED2) (SEQ ID NO: 189) operably linked to a morphogenic gene. The present disclosure provides for nucleic acid molecules comprising at least one of the nucleotide sequences set forth in SEQ ID NOS: 1-59, 108-110, 124-126, 149-152, and 189 and fragments and variants thereof operably linked to a morphogenic gene. The compositions further comprise expression cassettes, DNA constructs, and vectors comprising nucleic acid molecules comprising a nucleotide sequence of at least one of the nucleotide sequences set forth in SEQ ID NOS: 1-59, 108-110, 124-126, 149-152, and 189 operably linked to a morphogenic gene.

As used herein the term “tissue-preferred promoter disclosed herein” means a nucleotide sequence selected from the group consisting of at least one of SEQ ID NOS: 1-59, 108-110, 124-126, 149-152, and 189, wherein the nucleotide sequence initiates transcription in a plant cell; a nucleotide sequence that is at least 95% identical to at least one of SEQ ID NOS: 1-59, 108-110, 124-126, 149-152, and 189, wherein the nucleotide sequence initiates transcription in a plant cell; a nucleotide sequence having at least 70% identity to at least one of SEQ ID NOS: 1-59, 108-110, 124-126, 149-152, and 189, wherein the nucleotide sequence initiates transcription in a plant cell; a fragment or variant of the nucleotide sequence of at least one of SEQ ID NOS: 1-59, 108-110, 124-126, 149-152, and 189, wherein the fragment or variant initiates transcription in a plant cell; at least 100 contiguous nucleotides of a nucleotide sequence of at least one of SEQ ID NOS: 1-59, 108-110, 124-126, 149-152, and 189, wherein the at least 100 contiguous nucleotides of the nucleotide sequence initiates transcription in a plant cell; and at least a 100-bp fragment of the nucleotide sequence of at least one of SEQ ID NOS: 1-59, 108-110, 124-126, 149-152, and 189, wherein the at least a 100-bp fragment of the nucleotide sequence initiates transcription in a plant cell.

As used herein, the term “morphogenic gene” means a gene that when ectopically expressed stimulates formation of a somatically-derived structure that can produce a plant. More precisely, ectopic expression of the morphogenic gene stimulates the de novo formation of a somatic embryo or an organogenic structure, such as a shoot meristem, that can produce a plant. This stimulated de novo formation occurs either in the cell in which the morphogenic gene is expressed, or in a neighboring cell. A morphogenic gene can be a transcription factor that regulates expression of other genes, or a gene that influences hormone levels in a plant tissue, both of which can stimulate morphogenic changes. A morphogenic gene may be stably incorporated into the genome of a plant or it may be transiently expressed. A tissue-preferred promoter of the disclosure is used to express a morphogenic gene involved in plant metabolism, organ development, stem cell development, cell growth stimulation, organogenesis, regeneration, somatic embryogenesis initiation, accelerated somatic embryo maturation, initiation and/or development of the apical meristem, initiation and/or development of shoot meristem, initiation and/or development of shoots, or a combination thereof, such as WUS/WOX genes (WUS1, WUS2, WUS3, WOX2A, WOX4, WOX5, or WOX9) see U.S. Pat. Nos. 7,348,468 and 7,256,322 and United States Patent Application publications 20170121722 and 20070271628; Laux et al. (1996) Development 122:87-96; and Mayer et al. (1998) Cell 95:805-815; van der Graaff et al., 2009, Genome Biology 10:248; Dolzblasz et al., 2016, Mol. Plant 19:1028-39. Modulation of WUS/WOX is expected to modulate plant and/or plant tissue phenotype including plant metabolism, organ development, stem cell development, cell growth stimulation, organogenesis, regeneration, somatic embryogenesis initiation, accelerated somatic embryo maturation, initiation and/or development of the apical meristem, initiation and/or development of shoot meristem, initiation and/or development of shoots, or a combination thereof. Expression of Arabidopsis WUS can induce stem cells in vegetative tissues, which can differentiate into somatic embryos (Zuo, et al. (2002) Plant J 30:349-359). Also of interest in this regard would be a MYB118 gene (see U.S. Pat. No. 7,148,402), MYB115 gene (see Wang et al. (2008) Cell Research 224-235), a BABYBOOM gene (BBM; see Boutilier et al. (2002) Plant Cell 14:1737-1749), or a CLAVATA gene (see, for example, U.S. Pat. No. 7,179,963).

Morphogenic genes useful in the present disclosure include, but are not limited to, WUS/WOX genes known in the art as well as those disclosed herein. Morphogenic genes include but are not limited to the WUS/WOX genes disclosed herein including AT-WUS (SEQ ID NO: 60), LJ-W (SEQ ID NO: 62), GM-W (SEQ ID NO: 64), CS-WUS (SEQ ID NO: 66), CR-WUS (SEQ ID NO: 68), AA-WUS (SEQ ID NO: 70), RS-WUS (SEQ ID NO: 72), BN-WUS (SEQ ID NO: 74), BO-WU (SEQ ID NO: 76), HA-WUS (SEQ ID NO: 78), PT-WUS (SEQ ID NO: 80), VV-WUS (SEQ ID NO: 82), AT-WUS (soy optimized) (SEQ ID NO: 84), LJ-WUS (soy optimized) (SEQ ID NO: 86), MT-WUS (soy optimized) (SEQ ID NO: 88), PY-WUS (soy optimized) (SEQ ID NO: 90), PV-WUS (soy optimized) (SEQ ID NO: 92), ZM-WUS1 (SEQ ID NO: 94), ZM-WUS2 (SEQ ID NO: 96), ZM-WUS3 (SEQ ID NO: 98), ZM-WOX2A (SEQ ID NO: 100), ZM-WOX4 (SEQ ID NO: 102), ZM-WOX5A (SEQ ID NO: 104), ZM-WOX9 (SEQ ID NO: 106), GG-WUS (SEQ ID NO: 127), MD-WUS (SEQ ID NO: 129), ME-WUS (SEQ ID NO: 131), KF-WUS (SEQ ID NO: 133), GH-WUS (SEQ ID NO: 135), ZOSMA-WUS (SEQ ID NO: 137), AMBTR-WUS (SEQ ID NO: 139), AC-WUS (SEQ ID NO: 141), AH-WUS (SEQ ID NO: 143), CUCSA-WUS (SEQ ID NO: 145), and PINTA-WUS (SEQ ID NO: 147).

The WUS/WOX genes disclosed herein encode WUS/WOX homeobox polypeptides including AT-WUS (SEQ ID NO: 61), LJ-W (SEQ ID NO: 63), GM-W (SEQ ID NO: 65), CS-WUS (SEQ ID NO: 67), CR-WUS (SEQ ID NO: 69), AA-WUS (SEQ ID NO: 71), RS-WUS (SEQ ID NO: 73), BN-WUS (SEQ ID NO: 75), BO-WU (SEQ ID NO: 77), HA-WUS (SEQ ID NO: 79), PT-WUS (SEQ ID NO: 81), VV-WUS (SEQ ID NO: 83), AT-WUS (SEQ ID NO: 85), LJ-WUS (SEQ ID NO: 87), MT-WUS (SEQ ID NO: 89), PY-WUS (SEQ ID NO: 91), PV-WUS (SEQ ID NO: 93), ZM-WUS1 (SEQ ID NO: 95), ZM-WUS2 (SEQ ID NO: 97), ZM-WUS3 (SEQ ID NO: 99), ZM-WOX2A (SEQ ID NO: 101), ZM-WOX4 (SEQ ID NO: 103), ZM-WOX5A (SEQ ID NO: 105), ZM-WOX9 (SEQ ID NO: 107), GG-WUS (SEQ ID NO: 128), MD-WUS (SEQ ID NO: 130), ME-WUS (SEQ ID NO: 132), KF-WUS (SEQ ID NO: 134), GH-WUS (SEQ ID NO: 136), ZOSMA-WUS (SEQ ID NO: 138), AMBTR-WUS (SEQ ID NO: 130), AC-WUS (SEQ ID NO: 142), AH-WUS (SEQ ID NO: 144), CUCSA-WUS (SEQ ID NO: 146), and PINTA-WUS (SEQ ID NO: 148).

The WUS/WOX genes disclosed herein include those encoding a WUS/WOX homeobox polypeptide, wherein the WUS/WOX homeobox polypeptide comprises an amino acid sequence of any of SEQ ID NOS: 61, 63, 65, 67, 69, 71, 73, 75, 77, 79, 81, 83, 85, 87, 89, 91, 93, 95, 97, 99, 101, 103, 105, 107, 128, 130, 132, 134, 136, 138, 140, 142, 144, 146, or 148; or wherein the WUS/WOX homeobox polypeptide is encoded by a nucleotide sequence of any of SEQ ID NOS: 60, 62, 64, 66, 68, 70, 72, 74, 76, 78, 80, 82, 84, 86, 88, 90, 92, 94, 96, 98, 100, 102, 104, 106, 127, 129, 131, 133, 135, 137, 139, 141, 143, 145, or 147.

Other morphogenic genes useful in the present disclosure include, but are not limited to, LEC1 (Lotan et al., 1998, Cell 93:1195-1205), LEC2 (Stone et al., 2008, PNAS 105:3151-3156; Belide et al., 2013, Plant Cell Tiss. Organ Cult 113:543-553), KN1/STM (Sinha et al., 1993. Genes Dev 7:787-795), the IPT gene from Agrobacterium (Ebinuma and Komamine, 2001, In vitro Cell. Dev Biol-Plant 37:103-113), MONOPTEROS-DELTA (Ckurshumova et al., 2014, New Phytol. 204:556-566), the Agrobacterium AV-6b gene (Wabiko and Minemura 1996, Plant Physiol. 112:939-951), the combination of the Agrobacterium IAA-h and IAA-m genes (Endo et al., 2002, Plant Cell Rep., 20:923-928), the Arabidopsis SERK gene (Hecht et al., 2001, Plant Physiol. 127:803-816), the Arabiopsis AGL15 gene (Harding et al., 2003, Plant Physiol. 133:653-663).

As used herein, the term “transcription factor” means a protein that controls the rate of transcription of specific genes by binding to the DNA sequence of the promoter and either up-regulating or down-regulating expression. Examples of transcription factors, which are also morphogenic genes, include members of the AP2/EREBP family (including the BBM (ODP2), plethora and aintegumenta sub-families, CAAT-box binding proteins such as LEC1 and HAP3, and members of the MYB, bHLH, NAC, MADS, bZIP and WRKY families.

In an aspect, the recombinant expression cassette or construct comprises a nucleotide sequence encoding a WUS/WOX homeobox polypeptide. In various aspects, expression of a nucleotide sequence encoding a WUS/WOX homeobox occurs for from about 21 to about 28 days after initiation of transformation.

In an aspect, the nucleotide sequence encoding the WUS/WOX homeobox polypeptide can be targeted for excision by a site-specific recombinase. Thus, the expression of the nucleotide sequence encoding the WUS/WOX homeobox polypeptide can be controlled by excision at a desired time post-transformation. It is understood that when a site-specific recombinase is used to control the expression of the nucleotide sequence encoding the WUS/WOX homeobox polypeptide, the expression construct comprises appropriate site-specific excision sites flanking the polynucleotide sequences to be excised, e.g., Cre lox sites if Cre recombinase is utilized. It is not necessary that the site-specific recombinase be co-located on the expression construct comprising the nucleotide sequence encoding the WUS/WOX homeobox polypeptide. However, in an aspect, the expression construct further comprises a nucleotide sequence encoding a site-specific recombinase.

The site-specific recombinase used to control expression of the nucleotide sequence encoding the WUS/WOX homeobox polypeptide can be chosen from a variety of suitable site-specific recombinases. For examples, in various aspects, the site-specific recombinase is FLP, FLPe, KD, Cre, SSV1, lambda Int, phi C31 Int, HK022, R, B2 (Nern et al., (2011) PNAS Vol. 108, No. 34 pp 14198-14203), B3 (Nern et al., (2011) PNAS Vol. 108, No. 34 pp 14198-14203), Gin, Tn1721, CinH, ParA, Tn5053, Bxb1, TP907-1, or U153. The site-specific recombinase can be a destabilized fusion polypeptide. The destabilized fusion polypeptide can be TETR(G17A)˜CRE or ESR(G17A)˜CRE.

In an aspect, the nucleotide sequence encoding a site-specific recombinase is operably linked to a constitutive promoter, an inducible promoter, a tissue-specific promoter, or a developmentally-regulated promoter. Suitable constitutive promoters, inducible promoters, tissue-specific promoters, and developmentally-regulated promotersinclude UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, ZM-ADF PRO (ALT2), AXIG1, DR5, XVE, GLB1, OLE, LTP2 (Kalla et al., 1994. Plant J. 6:849-860 and U.S. Pat. No. 5,525,716), HSP17.7, HSP26, HSP18A, AT-HSP811, AT-HSP811L, GM-HSP173B, promoters activated by tetracycline, ethamethsulfuron or chlorsulfuron, PLTP, PLTP1, PLTP2, PLTP3, SDR, LGL, LEA-14A, or LEA-D34.

In an aspect, the chemically inducible promoter operably linked to the site-specific recombinase is XVE. The chemically-inducible promoter can be repressed by the tetraycline repressor (TETR), the ethametsulfuron repressor (ESR), or the chlorsulfuron repressor (CR), and de-repression occurs upon addition of tetracycline-related or sulfonylurea ligands. The repressor can be TETR and the tetracycline-related ligand is doxycycline or anhydrotetracycline. (Gatz, C., Frohberg, C. and Wendenburg, R. (1992) Stringent repression and homogeneous de-repression by tetracycline of a modified CaMV 35S promoter in intact transgenic tobacco plants, Plant J. 2, 397-404). Alternatively, the repressor can be ESR and the sulfonylurea ligand is ethametsulfuron, chlorsulfuron, metsulfuron-methyl, sulfometuron methyl, chlorimuron ethyl, nicosulfuron, primisulfuron, tribenuron, sulfosulfuron, trifloxysulfuron, foramsulfuron, iodosulfuron, prosulfuron, thifensulfuron, rimsulfuron, mesosulfuron, or halosulfuron (US20110287936 incorporated herein by reference in its entirety).

In an aspect, when the expression construct comprises site-specific recombinase excision sites, the nucleotide sequence encoding the WUS/WOX homeobox polypeptide can be operably linked to an auxin inducible promoter, a developmentally regulated promoter, a tissue-specific promoter, or a constitutive promoter. Exemplary auxin inducible promoters, developmentally regulated promoters, tissue-specific promoters, and constitutive promoters useful in this context include UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, ZM-ADF PRO (ALT2), AXIG1 (U.S. Pat. No. 6,838,593 incorporated herein by reference in its entirety), DR5, XVE, GLB1, OLE, LTP2, HSP17.7, HSP26, HSP18A, AT-HSP811 (Takahashi, T, et al., (1992) Plant Physiol. 99 (2): 383-390), AT-HSP811L (Takahashi, T, et al., (1992) Plant Physiol. 99 (2): 383-390), GM-HSP173B (Schöffl, F., et al. (1984) EMBO J. 3(11): 2491-2497), promoters activated by tetracycline, ethamethsulfuron or chlorsulfuron, PLTP, PLTP1, PLTP2, PLTP3, SDR, LGL, LEA-14A, LEA-D34, at least one of SEQ ID NOS: 1-59, 108-110, 124-126, 149-152, and 189, a nucleotide sequence that is at least 95% identical to at least one of SEQ ID NOS: 1-59, 108-110, 124-126, 149-152, and 189, a nucleotide sequence that is at least 70% identical to at least one of SEQ ID NOS: 1-59, 108-110, 124-126, 149-152, and 189, a fragment or variant of at least one of SEQ ID NOS: 1-59, 108-110, 124-126, 149-152, and 189, or at least a 100-bp fragment of at least one of SEQ ID NOS: 1-59, 108-110, 124-126, 149-152, and 189.

The appropriate duration for expression of the nucleotide sequence encoding a WUS/WOX homeobox polypeptide can be achieved by use of a tissue-preferred promter disclosed hereinincluding, but not limited to at least one of SEQ ID NOS: 1-59, 108-110, 124-126, 149-152, and 189, a nucleotide sequence that is at least 95% identical to at least one of SEQ ID NOS: 1-59, 108-110, 124-126, 149-152, and 189, a nucleotide sequence that is at least 70% identical to at least one of SEQ ID NOS: 1-59, 108-110, 124-126, 149-152, and 189, a fragment or variant of at least one of SEQ ID NOS: 1-59, 108-110, 124-126, 149-152, and 189, or at least a 100-bp fragment of at least one of SEQ ID NOS: 1-59, 108-110, 124-126, 149-152, and 189. When using a morphogenic gene cassette and a trait gene cassette to produce transgenic plants it is desirable to have the ability to segregate the morphogenic gene locus away from the trait gene locus in co-transformed plants to provide transgenic plants containing only the trait gene. This can be accomplished using an Agrobacterium tumefaciens two T-DNA binary system, with two variations on this general theme (see Miller et al., 2002). For example, in the first, a two T-DNA vector, where expression cassettes for morphogenic genes and herbicide selection (i.e. HRA) are contained within a first T-DNA and the trait gene cassette is contained within a second T-DNA, where both T-DNA's reside on a single binary vector. When a plant cell is transformed by an Agrobacterium containing the two T-DNA plasmid, a high percentage of transformed cells contain both T-DNA's that have integrated into different genomic locations (for example, onto different chromosomes). In the second method, for example, two Agrobacterium strains, each containing one of the two T-DNA's (either the morphogenic gene T-DNA or the trait gene T-DNA), are mixed together in a ratio, and the mixture is used for transformation. After transformation using this mixed Agrobacterium method, it is observed at a high frequency that recovered transgenic events contain both T-DNA's, often at separate genomic locations. For both co-transformation methods, it is observed that in a large proportion of the produced transgenic events, the two T-DNA loci segregate independently and progeny T1 plants can be readily identified in which the T-DNA loci have segregated away from each other, resulting in the recovery of progeny seed that contain the trait genes with no morphogenic genes/herbicide genes. See, Miller et al. Transgenic Res 11(4):381-96.

The methods provided herein rely upon the use of bacteria-mediated and/or biolistic-mediated gene transfer to produce regenerable plant cells having an incorporated nucleotide sequence of interest. Bacterial strains useful in the methods of the disclosure include, but are not limited to, a disarmed Agrobacteria, an Ochrobactrum bacteria or a Rhizobiaceae bacteria. In an aspect, the different bacterial strains are selected from (i) a disarmed Agrobacteria and an Ochrobactrum bacteria, (ii) a disarmed Agrobacteria and a Rhizobiaceae bacteria, and (iii) a Rhizobiaceae bacteria and an Ochrobactrum bacteria.

Disarmed Agrobacteria useful in the present methods include, but are not limited to, AGL-1, EHA105, GV3101, LBA4404, and LBA4404 THY-.

Ochrobactrum bacterial strains useful in the present methods include, but are not limited to, Ochrobactrum haywardense H1 NRRL Deposit B-67078, Ochrobactrum cytisi, Ochrobactrum daejeonense, Ochrobactrum oryzae, Ochrobactrum tritici LBNL124-A-10, HTG3-C-07, Ochrobactrum pecoris, Ochrobactrum ciceri, Ochrobactrum gallinifaecis, Ochrobactrum grignonense, Ochrobactrum guangzhouense, Ochrobactrum haematophilum, Ochrobactrum intermedium, Ochrobactrum lupini, Ochrobactrum pituitosum, Ochrobactrum pseudintermedium, Ochrobactrum pseudogrignonense, Ochrobactrum rhizosphaerae, Ochrobactrum thiophenivorans, and Ochrobactrum tritici.

Rhizobiaceae bacterial strains useful in the present methods include, but are not limited to, Rhizobium lusitanum, Rhizobium rhizogenes, Agrobacterium rubi, Rhizobium multihospitium, Rhizobium tropici, Rhizobium miluonense, Rhizobium leguminosarum, Rhizobium leguminosarum bv. trifolii, Rhizobium leguminosarum bv. phaseoli, Rhizobium leguminosarum. bv. viciae, Rhizobium leguminosarum Madison, Rhizobium leguminosarum USDA2370, Rhizobium leguminosarum USDA2408, Rhizobium leguminosarum USDA2668, Rhizobium leguminosarum 2370G, Rhizobium leguminosarum 2370LBA, Rhizobium leguminosarum 2048G, Rhizobium leguminosarum 2048LBA, Rhizobium leguminosarum bv. phaseoli 2668G, Rhizobium leguminosarum bv. phaseoli 2668LBA, Rhizobium leguminosarum RL542C, Rhizobium etli USDA 9032, Rhizobium etli bv. phaseoli, Rhizobium endophyticum, Rhizobium tibeticum, Rhizobium etli, Rhizobium pisi, Rhizobium phaseoli, Rhizobium fabae, Rhizobium hainanense, Arthrobacter viscosus, Rhizobium alamii, Rhizobium mesosinicum, Rhizobium sullae, Rhizobium indigoferae, Rhizobium gallicum, Rhizobium yanglingense, Rhizobium mongolense, Rhizobium oryzae, Rhizobium loessense, Rhizobium tubonense, Rhizobium cellulosilyticum, Rhizobium soli, Neorhizobium galegae, Neorhizobium vignae, Neorhizobium huautlense, Neorhizobium alkalisoli, Aureimonas altamirensis, Aureimonas frigidaquae, Aureimonas ureilytica. Aurantimonas coralicida, Fulvimarina pelagi, Martelella mediterranea, Allorhizobium undicola, Allorhizobium vitis, Allorhizobium borbor, Beijerinckia fluminensis, Agrobacterium larrymoorei, Agrobacterium radiobacter, Rhizobium selenitireducens corrig. Rhizobium rosettiformans, Rhizobium daejeonense, Rhizobium aggregatum, Pararhizobium capsulatum, Pararhizobium giardinii, Ensifer mexicanus, Ensifer terangae, Ensifer saheli, Ensifer kostiensis, Ensifer kummerowiae, Ensifer fredii, Sinorhizobium americanum, Ensifer arboris, Ensifer garamanticus, Ensifer meliloti, Ensifer numidicus, Ensifer adhaerens, Sinorhizobium sp., Sinorhizobium meliloti SD630, Sinorhizobium meliloti USDA1002, Sinorhizobium fredii USDA205, Sinorhizobium fredii SF542G, Sinorhizobium fredii SF4404, and Sinorhizobium fredii SM542C. See U.S. Pat. No. 9,365,859 incorporated herin by reference in its entirety.

In an aspect, the first bacterial strain and the second bacterial strain are present in a 50:50 ratio. In an aspect, the first bacterial strain and the second bacterial strain are present in a 25:75 ratio. In an aspect, the first bacterial strain and the second bacterial strain are present in a 10:90 ratio. In an aspect, the first bacterial strain and the second bacterial strain are present in a 5:95 ratio. In an aspect, the first bacterial strain and the second bacterial strain are present in a 1:99 ratio. In an aspect, the first bacterial strain and the second bacterial strain are different bacterial strains.

The promoters of the present disclosure include nucleotide sequences that allow initiation of transcription in a plant. In specific aspects, the promoters allow initiation of transcription in a tissue-preferred manner. Constructs of the disclosure comprise a tissue-preferred promoter disclosed herein operably linked to a morphogenic gene.

The tissue-preferred promoters disclosed herein also find use in the construction of expression cassettes or vectors for subsequent expression of a heterologous polynucleotide or a polynucleotide of interest in a plant of interest or as probes for the isolation of other promoters. The present disclosure provides for isolated DNA constructs comprising the promoter nucleotide sequences set forth in at least one of SEQ ID NOS: 1-59, 108-110, 124-126, 149-152, and 189 operably linked to morphogenic gene and optionally further comprising a heterologous polynucleotide or polynucleotide of interest.

Aspects of the disclosure include a nucleic acid molecule comprising a promoter having a nucleotide sequence selected from the group consisting of: at least one of SEQ ID NOS: 1-59, 108-110, 124-126, 149-152, and 189, wherein the nucleotide sequence initiates transcription in a plant cell; a nucleotide sequence that is at least 95% identical to at least one of SEQ ID NOS: 1-59, 108-110, 124-126, 149-152, and 189, wherein the nucleotide sequence initiates transcription in a plant cell; a nucleotide sequence having at least 70% identity to at least one of SEQ ID NOS: 1-59, 108-110, 124-126, 149-152, and 189, wherein the nucleotide sequence initiates transcription in a plant cell; a fragment or variant of the nucleotide sequence of at least one of SEQ ID NOS: 1-59, 108-110, 124-126, 149-152, and 189, wherein the fragment or variant initiates transcription in a plant cell; at least 100 contiguous nucleotides of a nucleotide sequence of at least one of SEQ ID NOS: 1-59, 108-110, 124-126, 149-152, and 189, wherein the at least 100 contiguous nucleotides of the nucleotide sequence initiates transcription in a plant cell; and at least a 100-bp fragment of the nucleotide sequence of at least one of SEQ ID NOS: 1-59, 108-110, 124-126, 149-152, and 189, wherein the at least a 100-bp fragment of the nucleotide sequence initiates transcription in a plant cell; wherein the promoter is operably linked to a morphogenic gene and optionally further comprising a heterologous polynucleotide or a polynucleotide of interest. Also embodied is an expression cassette comprising the promoter containing the nucleic acid, a vector comprising the expression cassette, and a plant cell comprising the expression cassette. Further aspects include a plant cell or plant wherein the expression cassette is transiently expressed or stably integrated into the genome of the plant cell or plant, whether monocot or dicot plant cells or plants, and a plant comprising the described expression cassette, whether monocot or dicot plant, the monocot or dicot plant cell or plant including maize, alfalfa, sorghum, rice, millet, soybean, wheat, cotton, sunflower, barley, oats, rye, flax, sugarcane, banana, cassava, common bean, cowpea, tomato, potato, beet, grape, Eucalyptus, poplar, pine, douglas fir, citrus, papaya, cacao, cucumber, apple, Capsicum, bamboo, Triticale, melon, and Brassica.

Also embodied is a plant with the described expression cassette stably incorporated into the genome of the plant, a seed of the plant, wherein the seed comprises the expression cassette. Further embodied is a plant wherein a gene or gene product of a heterologous polynucleotide or a polynucleotide of interest confers nutritional enhancement, increased yield, abiotic stress tolerance, drought tolerance, cold tolerance, herbicide tolerance, pest resistance, pathogen resistance, insect resistance, nitrogen use efficiency (NUE), disease resistance, or an ability to alter a metabolic pathway. A plant wherein expression of a heterologous polynucleotide or a polynucleotide of interest alters the phenotype of said plant is also embodied. Also embodied is an expression cassette comprising a recombinant polynucleotide comprising a functional fragment having promoter activity, wherein the fragment is derived from a nucleotide sequence selected from the group consisting of SEQ ID NOS: 1-59, 108-110, 124-126, 149-152, and 189, wherein the functional fragment having promoter activity is operably linked to a morphogenic gene.

The disclosure encompasses isolated or substantially purified nucleic acid compositions. An “isolated” or “purified” nucleic acid molecule or biologically active portion thereof is substantially free of other cellular material or culture medium when produced by recombinant techniques or substantially free of chemical precursors or other chemicals when chemically synthesized. An “isolated” nucleic acid is substantially free of sequences (including protein encoding sequences) that naturally flank the nucleic acid (i.e., sequences located at the 5′ and 3′ ends of the nucleic acid) in the genomic DNA of the organism from which the nucleic acid is derived. For example, in various aspects, the isolated nucleic acid molecule can contain less than about 5 kb, 4 kb, 3 kb, 2 kb, 1 kb, 0.5 kb or 0.1 kb of nucleotide sequences that naturally flank the nucleic acid molecule in genomic DNA of the cell from which the nucleic acid is derived. The sequences of the disclosure may be isolated from the 5′ untranslated region flanking their respective transcription initiation sites.

Fragments and variants of the disclosed promoter nucleotide sequences are also encompassed by the present disclosure. Fragments and variants of the promoter sequences of at least one of SEQ ID NOS: 1-59, 108-110, 124-126, 149-152, and 189 may be used in the DNA constructs of the disclosure. As used herein, the term “fragment” refers to a portion of the nucleic acid sequence. Fragments of promoter sequences retain the biological activity of initiating transcription, such as driving transcription in a constitutive manner. Alternatively, fragments of a nucleotide sequence that are useful as hybridization probes may not necessarily retain biological activity. Fragments of a nucleotide sequence for the promoters disclosed herein may range from at least about 20, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, 2525, 2550, 2575, 2600, 2625, 2650, 2675, 2700, 2725, 2750, 2775, 2800, 2825, 2850, 2875, 2900, 2925, 2950, 2975, 3000, 3025, 3050, 3075, 3100, 3125, 3150, 3175, 3200, 3225, 3250, 3275, 3300, 3325, 3350, 3375, 3400, 3425, 3450, 3475, 3500, 3525, 3550, 3575, 3600, 3625, 3650, 3675, 3700, 3725, 3750, 3775, 3800, 3825, 3850, 3875, 3900, 3925, 3950, 3975, 4000, 4025, 4050, 4075, 4100, 4125, 4150, 4175, 4200, 4225, 4250, 4275, 4300, 4325, 4350, 4375, 4400, 4425, 4450, 4475, 4500, 4525, 4550, 4575, 4600, 4625, 4650, 4675, 4700, 4725, 4750, 4775, 4800, 4825, 4850, 4875, 4900, 4925, 4950, 4975, 5000, 5025, 5050, 5075, 5100, 5125, 5150, 5175, 5200, 5225, 5250, 5275, 5300, 5325, 5350, 5375, 5400, 5425, 5450, 5475, 5500, 5525, 5550, 5575, 5600, 5625, 5650, 5675, 5700, 5725, 5750, 5775, 5800, 5825, 5850, 5875, 5900, 5925, 5950, 5975, 6000, 6025, 6050, 6075, 6100, 6125, 6150, 6175, 6200, or 6225 nucleotides, and up to the full length of at least one of SEQ ID NOS: 1-59, 108-110, 124-126, 149-152, and 189. A biologically active portion of a promoter can be prepared by isolating a portion of the promoter sequences of the disclosure, and assessing the transcription activity of the portion.

As used herein, the term “variants” is means sequences having substantial similarity with a promoter sequence disclosed herein. A variant comprises a deletion and/or addition of one or more nucleotides at one or more internal sites within the native polynucleotide and/or a substitution of one or more nucleotides at one or more sites in the native polynucleotide. As used herein, a “native” nucleotide sequence comprises a naturally occurring nucleotide sequence. For nucleotide sequences, naturally occurring variants can be identified with the use of well-known molecular biology techniques, such as, for example, with polymerase chain reaction (PCR) and hybridization techniques as outlined herein.

Variant nucleotide sequences also include synthetically derived nucleotide sequences, such as those generated, for example, by using site-directed mutagenesis. Generally, variants of a nucleotide sequence disclosed herein will have at least 40%, 50%, 60%, 65%, 70%, 75%, 80%, 85%, 90%, 91%, 92%, 93%, 94%, to 95%, 96%, 97%, 98%, 99% or more sequence identity to that nucleotide sequence as determined by sequence alignment programs described elsewhere herein using default parameters. Biologically active variants of a nucleotide sequence disclosed herein are also encompassed. Biologically active variants include, for example, the native promoter sequences of a nucleotide sequence disclosed herein having one or more nucleotide substitutions, deletions or insertions. Promoter activity may be measured by using techniques such as Northern blot analysis, reporter activity measurements taken from transcriptional fusions, and the like. See, for example, Sambrook, et al., (1989) Molecular Cloning: A Laboratory Manual (2d ed., Cold Spring Harbor Laboratory Press, Cold Spring Harbor, N.Y.), hereinafter “Sambrook,” herein incorporated by reference in its entirety. Alternatively, levels of a reporter gene such as green fluorescent protein (GFP) or yellow fluorescent protein (YFP) or the like produced under the control of a promoter fragment or variant can be measured. See, for example, Matz et al. (1999) Nature Biotechnology 17:969-973; U.S. Pat. No. 6,072,050, herein incorporated by reference in its entirety; Nagai, et al., (2002) Nature Biotechnology 20(1):87-90. Variant nucleotide sequences also encompass sequences derived from a mutagenic and recombinogenic procedure such as DNA shuffling. With such a procedure, one or more different nucleotide sequences for the promoter can be manipulated to create a new promoter. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer, (1994) Proc. Natl. Acad. Sci. USA 91:10747-10751; Stemmer, (1994) Nature 370:389 391; Crameri, et al., (1997) Nature Biotech. 15:436-438; Moore, et al., (1997)J Mol. Biol. 272:336-347; Zhang, et al., (1997) Proc. Natl. Acad. Sci. USA 94:4504-4509; Crameri, et al., (1998) Nature 391:288-291 and U.S. Pat. Nos. 5,605,793 and 5,837,458, herein incorporated by reference in their entirety.

Methods for mutagenesis and nucleotide sequence alterations are well known in the art. See, for example, Kunkel, (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein, herein incorporated by reference in their entirety.

The nucleotide sequences of the disclosure can be used to isolate corresponding sequences from other organisms, particularly other plants, more particularly other monocots or dicots. In this manner, methods such as PCR, hybridization and the like can be used to identify such sequences based on their sequence homology to the sequences set forth herein. Sequences isolated based on their sequence identity to the entire sequences set forth herein or to fragments thereof are encompassed by the present disclosure.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in, Sambrook, supra. See also, Innis, et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York), herein incorporated by reference in their entirety. Known methods of PCR include, but are not limited to, methods using paired primers, nested primers, single specific primers, degenerate primers, gene-specific primers, vector-specific primers, partially-mismatched primers and the like.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides and may be labeled with a detectable group such as ³²P or any other detectable marker. Thus, for example, probes for hybridization can be made by labeling synthetic oligonucleotides based on the promoters of the disclosure. Methods for preparation of probes for hybridization and for construction of genomic libraries are generally known in the art and are disclosed in Sambrook, supra.

For example, an entire promoter sequence disclosed herein, or one or more portions thereof, may be used as a probe capable of specifically hybridizing to corresponding promoter sequences and messenger RNAs. To achieve specific hybridization under a variety of conditions, such probes include sequences that are unique among promoter sequences and are generally at least about 10 nucleotides in length or at least about 20 nucleotides in length. Such probes may be used to amplify corresponding promoter sequences from a chosen plant by PCR. This technique may be used to isolate additional coding sequences from a desired organism or as a diagnostic assay to determine the presence of coding sequences in an organism. Hybridization techniques include hybridization screening of plated DNA libraries (either plaques or colonies, see, for example, Sambrook, supra).

Hybridization of such sequences may be carried out under stringent conditions. The terms “stringent conditions” or “stringent hybridization conditions” are intended to mean conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, optimally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C. and a wash in 1 times to 2 times SSC (20 times SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C. and a wash in 0.5 times to 1 times SSC at 55 to 60° C. Exemplary high stringency conditions include hybridization in 50% formamide, 1 M NaCl, 1% SDS at 37° C., and a final wash in 0.1 times SSC at 60 to 65° C. for a duration of at least 30 minutes. Duration of hybridization is generally less than about 24 hours, usually about 4 to about 12 hours. The duration of the wash time will be at least a length of time sufficient to reach equilibrium.

Specificity is typically the function of post-hybridization washes, the critical factors being the ionic strength and temperature of the final wash solution. For DNA-DNA hybrids, the thermal melting point (Tₘ) can be approximated from the equation of Meinkoth and Wahl, (1984) Anal. Biochem 138:267 284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching, thus, Tₘ, hybridization, and/or wash conditions can be adjusted to hybridize to sequences of the desired identity. For example, if sequences with 90% identity are sought, the Tₘ can be decreased 10° C. Generally, stringent conditions are selected to be about 5° C. lower than the Tₘ for the specific sequence and its complement at a defined ionic strength and pH. However, severely stringent conditions can utilize a hybridization and/or wash at 1, 2, 3 or 4° C. lower than the Tₘ; moderately stringent conditions can utilize a hybridization and/or wash at 6, 7, 8, 9 or 10° C. lower than the Tₘ; low stringency conditions can utilize a hybridization and/or wash at 11, 12, 13, 14, 15 or 20° C. lower than the Tₘ. Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen, (1993) Laboratory Techniques in Biochemistry and Molecular Biology—Hybridization with Nucleic Acid Probes, Part I, Chapter 2 (Elsevier, New York); and Ausubel, et al., eds. (1995) Current Protocols in Molecular Biology, Chapter 2 (Greene Publishing and Wiley-Interscience, New York), herein incorporated by reference in their entirety. See also, Sambrook supra. Thus, isolated sequences that have promoter activity and which hybridize under stringent conditions to the promoter sequences disclosed herein or to fragments thereof, are encompassed by the present disclosure.

In general, sequences that have promoter activity and hybridize to the promoter sequences disclosed herein will be at least 40% to 50% homologous, about 60%, 70%, 80%, 85%, 90%, 95% to 98% homologous or more with the disclosed sequences. That is, the sequence similarity of sequences may range, sharing at least about 40% to 50%, about 60% to 70%, and about 80%, 85%, 90%, 95% to 98% sequence similarity.

The following terms are used to describe the sequence relationships between two or more nucleic acids or polynucleotides: (a) “reference sequence”, (b) “comparison window”, (c) “sequence identity”, (d) “percentage of sequence identity” and (e) “substantial identity”.

As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” refers to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller, (1988) CABIOS 4:11-17; the algorithm of Smith, et al., (1981) Adv. Appl. Math. 2:482; the algorithm of Needleman and Wunsch, (1970) J. Mol. Biol. 48:443-453; the algorithm of Pearson and Lipman, (1988) Proc. Natl. Acad. Sci. 85:2444-2448; the algorithm of Karlin and Altschul, (1990) Proc. Natl. Acad. Sci. USA 872:264, modified as in Karlin and Altschul, (1993) Proc. Natl. Acad. Sci. USA 90:5873-5877, herein incorporated by reference in their entirety.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA and TFASTA in the GCG Wisconsin Genetics Software Package®, Version 10 (available from Accelrys Inc., 9685 Scranton Road, San Diego, Calif., USA). Alignments using these programs can be performed using the default parameters. The CLUSTAL program is well described by Higgins, et al., (1988) Gene 73:237-244; Higgins, et al., (1989) CABIOS 5:151-153; Corpet, et al., (1988) Nucleic Acids Res. 16:10881-90; Huang, et al., (1992) CABIOS 8:155-65; and Pearson, et al., (1994) Meth. Mol. Biol. 24:307-331, herein incorporated by reference in their entirety. The ALIGN program is based on the algorithm of Myers and Miller, (1988) supra. A PAM120 weight residue table, a gap length penalty of 12, and a gap penalty of 4 can be used with the ALIGN program when comparing amino acid sequences. The BLAST programs of Altschul, et al., (1990) J. Mol. Biol. 215:403, herein incorporated by reference in its entirety, are based on the algorithm of Karlin and Altschul, (1990) supra. BLAST nucleotide searches can be performed with the BLASTN program, score=100, word length=12, to obtain nucleotide sequences homologous to a nucleotide sequence encoding a protein of the disclosure. BLAST protein searches can be performed with the BLASTX program, score=50, word length=3, to obtain amino acid sequences homologous to a protein or polypeptide of the disclosure. To obtain gapped alignments for comparison purposes, Gapped BLAST (in BLAST 2.0) can be utilized as described in Altschul, et al., (1997) Nucleic Acids Res. 25:3389, herein incorporated by reference in its entirety. Alternatively, PSI-BLAST (in BLAST 2.0) can be used to perform an iterated search that detects distant relationships between molecules. See, Altschul, et al., (1997) supra. When utilizing BLAST, Gapped BLAST, PSI-BLAST, the default parameters of the respective programs (e.g., BLASTN for nucleotide sequences, BLASTX for proteins) can be used. See, the web site for the National Center for Biotechnology Information on the World Wide Web at ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. As used herein, “equivalent program” is any sequence comparison program that, for any two sequences in question, generates an alignment having identical nucleotide or amino acid residue matches and an identical percent sequence identity when compared to the corresponding alignment generated by GAP Version 10.

The GAP program uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package® for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the Quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package® is BLOSUM62 (see, Henikoff and Henikoff, (1989) Proc. Natl. Acad. Sci. USA 89:10915, herein incorporated by reference in its entirety).

As used herein, “sequence identity” or “identity” in the context of two nucleic acid or polypeptide sequences refers to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of one and a non-conservative substitution is given a score of zero, a conservative substitution is given a score between zero and one. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70% sequence identity, optimally at least 80%, more optimally at least 90% and most optimally at least 95%, compared to a reference sequence using an alignment program using standard parameters. One of skill in the art will recognize that these values can be appropriately adjusted to determine corresponding identity of proteins encoded by two nucleotide sequences by considering codon degeneracy, amino acid similarity, reading frame positioning and the like. Substantial identity of amino acid sequences for these purposes normally means sequence identity of at least 60%, 70%, 80%, 90% and at least 95%.

Another indication that nucleotide sequences are substantially identical is if two molecules hybridize to each other under stringent conditions. Generally, stringent conditions are selected to be about 5° C. lower than the Tₘ for the specific sequence at a defined ionic strength and pH. However, stringent conditions encompass temperatures in the range of about 1° C. to about 20° C. lower than the Tₘ, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

The tissue-preferred promoters disclosed herein, as well as variants and fragments thereof, are useful for genetic engineering of plants, e.g. to produce a transformed or transgenic plant, to express a phenotype of interest. As used herein, the terms “transformed plant” and “transgenic plant” refer to a plant that comprises within its genome a heterologous polynucleotide. Generally, the heterologous polynucleotide is stably integrated within the genome of a transgenic or transformed plant such that the polynucleotide is passed on to successive generations. The heterologous polynucleotide may be integrated into the genome alone or as part of a recombinant DNA construct. It is to be understood that as used herein the term “transgenic” includes any cell, cell line, callus, tissue, plant part or plant the genotype of which has been altered by the presence of a heterologous nucleic acid including those transgenics initially so altered as well as those created by sexual crosses or asexual propagation from the initial transgenic.

A transgenic “event” is produced by transformation of plant cells with a heterologous DNA construct, including a nucleic acid expression cassette that comprises a gene of interest, the regeneration of a population of plants resulting from the insertion of the transferred gene into the genome of the plant and selection of a plant characterized by insertion into a particular genome location. An event is characterized phenotypically by the expression of the inserted gene. At the genetic level, an event is part of the genetic makeup of a plant. The term “event” also refers to progeny produced by a sexual cross between the transformant and another plant wherein the progeny include the heterologous DNA.

The term “plant” refers to whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores.

Plant parts include differentiated and undifferentiated tissues including, but not limited to the following: roots, stems, shoots, leaves, pollen, seeds, tumor tissue and various forms of cells and culture (e.g., single cells, protoplasts, embryos and callus tissue). The plant tissue may be in a plant or in a plant organ, tissue or cell culture.

The present disclosure also includes plants obtained by any of the disclosed methods or compositions herein. The present disclosure also includes seeds from a plant obtained by any of the disclosed methods or compositions herein. The term “plant” refers to whole plants, plant organs (e.g., leaves, stems, roots, etc.), plant tissues, plant cells, plant parts, seeds, propagules, embryos and progeny of the same. Plant cells can be differentiated or undifferentiated (e.g. callus, undifferentiated callus, immature and mature embryos, immature zygotic embryo, immature cotyledon, embryonic axis, suspension culture cells, protoplasts, leaf, leaf cells, root cells, phloem cells and pollen). Plant cells include, without limitation, cells from seeds, suspension cultures, explants, immature embryos, embryos, zygotic embryos, somatic embryos, embryogenic callus, meristem, somatic meristems, organogenic callus, protoplasts, embryos derived from mature ear-derived seed, leaf bases, leaves from mature plants, leaf tips, immature influorescences, tassel, immature ear, silks, cotyledons, immature cotyledons, embryonic axes, meristematic regions, callus tissue, cells from leaves, cells from stems, cells from roots, cells from shoots, gametophytes, sporophytes, pollen and microspores. Plant parts include differentiated and undifferentiated tissues including, but not limited to, roots, stems, shoots, leaves, pollen, seeds, tumor tissue and various forms of cells in culture (e. g., single cells, protoplasts, embryos, and callus tissue). The plant tissue may be in a plant or in a plant organ, tissue, or cell culture. Grain is intended to mean the mature seed produced by commercial growers for purposes other than growing or reproducing the species. Progeny, variants and mutants of the regenerated plants are also included within the scope of the disclosure, provided these progeny, variants and mutants comprise the introduced polynucleotides.

The present disclosure may be used for transformation of any plant species, including, but not limited to, monocots and dicots including, but not limited to maize, alfalfa, sorghum, rice, millet, soybean, wheat, cotton, sunflower, barley, oats, rye, flax, sugarcane, banana, cassava, common bean, cowpea, tomato, potato, beet, grape, Eucalyptus, poplar, pine, douglas fir, citrus, papaya, cacao, cucumber, apple, Capsicum, bamboo, Triticale, melon, and Brassica. Monocots include, but are not limited to, barley, maize (corn), millet (e.g., pearl millet (Pennisetum glaucum), proso millet (Panicum miliaceum), foxtail millet (Setaria italica), finger millet (Eleusine coracana)), oats, rice, rye, Setaria sp., sorghum, triticale, or wheat, or leaf and stem crops, including, but not limited to, bamboo, marram grass, meadow-grass, reeds, ryegrass, sugarcane; lawn grasses, ornamental grasses, and other grasses such as switchgrass and turf grass. Alternatively, dicot plants used in the present disclosure, include, but are not limited to, kale, cauliflower, broccoli, mustard plant, cabbage, pea, clover, alfalfa, broad bean, tomato, peanut, cassava, soybean, canola, sunflower, safflower, tobacco, Arabidopsis, or cotton.

Higher plants, e.g., classes of Angiospermae and Gymnospermae may be used the present disclosure. Plants of suitable species useful in the present disclosure may come from the family Acanthaceae, Alliaceae, Alstroemeriaceae, Amaryllidaceae, Apocynaceae, Arecaceae, Asteraceae, Berberidaceae, Bixaceae, Brassicaceae, Bromeliaceae, Cannabaceae, Caryophyllaceae, Cephalotaxaceae, Chenopodiaceae, Colchicaceae, Cucurbitaceae, Dioscoreaceae, Ephedraceae, Erythroxylaceae, Euphorbiaceae, Fabaceae, Lamiaceae, Linaceae, Lycopodiaceae, Malvaceae, Melanthiaceae, Musaceae, Myrtaceae, Nyssaceae, Papaveraceae, Pinaceae, Plantaginaceae, Poaceae, Rosaceae, Rubiaceae, Salicaceae, Sapindaceae, Solanaceae, Taxaceae, Theaceae, and Vitaceae. Plants from members of the genus Abelmoschus, Abies, Acer, Agrostis, Allium, Alstroemeria, Ananas, Andrographis, Andropogon, Artemisia, Arundo, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Dianthus, Digitalis, Dioscorea, Elaeis, Ephedra, Erianthus, Erythroxylum, Eucalyptus, Festuca, Fragaria, Galanthus, Glycine, Gossypium, Helianthus, Hevea, Hordeum, Hyoscyamus, Jatropha, Lactuca, Linum, Lolium, Lupinus, Lycopersicon, Lycopodium, Manihot, Medicago, Mentha, Miscanthus, Musa, Nicotiana, Oryza, Panicum, Papaver, Parthenium, Pennisetum, Petunia, Phalaris, Phleum, Pinus, Poa, Poinsettia, Populus, Rauwolfia, Ricinus, Rosa, Saccharum, Salix, Sanguinaria, Scopolia, Secale, Solanum, Sorghum, Spartina, Spinacea, Tanacetum, Taxus, Theobroma, Triticosecale, Triticum, Uniola, Veratrum, Vinca, Vitis, and Zea may be used in the methods of the disclosure.

Plants important or interesting for agriculture, horticulture, biomass production (for production of liquid fuel molecules and other chemicals), and/or forestry may be used in the methods of the disclosure. Non-limiting examples include, for instance, Panicum virgatum (switchgrass), Miscanthus giganteus (miscanthus), Saccharum spp. (sugarcane, energycane), Populus balsamifera (poplar), cotton (Gossypium barbadense, Gossypium hirsutum), Helianthus annuus (sunflower), Medicago sativa (alfalfa), Beta vulgaris (sugarbeet), sorghum (Sorghum bicolor, Sorghum vulgare), Erianthus spp., Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Arundo donax (giant reed), Secale cereale (rye), Salix spp. (willow), Eucalyptus spp. (eucalyptus, including E. grandis (and its hybrids, known as “urograndis”), E. globulus, E. camaldulensis, E. tereticornis, E.viminalis, E. nitens, E. saligna and E. urophylla), Triticosecale spp. (triticum—wheat X rye), Bamboo, Carthamus tinctorius (safflower), Jatropha curcas (jatropha), Ricinus communis (castor), Elaeis guineensis (palm), Linum usitatissimum (flax), Manihot esculenta (cassava), Lycopersicon esculentum (tomato), Lactuca sativa (lettuce), Phaseolus vulgaris (green beans), Phaseolus limensis (lima beans), Lathyrus spp. (peas), Musa paradisiaca (banana), Solanum tuberosum (potato), Brassica spp. (B. napus (canola), B. rapa, B. juncea), Brassica oleracea (broccoli, cauliflower, brussel sprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Arachis hypogaea (peanuts), Ipomoea batatus (sweet potato), Cocos nucifera (coconut), Citrus spp. (citrus trees), Persea americana (avocado), fig (Ficus casica), guava (Psidium guajava), mango (Mangifera indica), olive (Olea europaea), Carica papaya (papaya), Anacardium occidentale (cashew), Macadamia integrifolia (macadamia tree), Prunus amygdalus (almond), Allium cepa (onion), Cucumis melo (musk melon), Cucumis sativus (cucumber), Cucumis scantalupensis (cantaloupe), Cucurbita maxima (squash), Cucurbita moschata (squash), Spinacea oleracea (spinach), Citrullus lanatus (watermelon), Abelmoschus esculentus (okra), Solanum melongena (eggplant), Cyamopsis tetragonoloba (guar bean), Ceratonia siliqua (locust bean), Trigonella foenum-graecum (fenugreek), Vigna radiata (mung bean), Vigna unguiculata (cowpea), Vicia faba (fava bean), Cicer arietinum (chickpea), Lens culinaris (lentil), Papaver somniferum (opium poppy), Papaver orientale, Taxus baccata, Taxus brevifolia, Artemisia annua, Cannabis sativa, Camptotheca acuminate, Catharanthus roseus, Vinca rosea, Cinchona officinalis, Colchicum autumnale, Veratrum californica., Digitalis lanata, Digitalis purpurea, Dioscorea spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana (achiote), Alstroemeria spp., Rosa spp. (rose), Rhododendron spp. (azalea), Macrophylla hydrangea (hydrangea), Hibiscus rosasanensis (hibiscus), Tulipa spp. (tulips), Narcissus spp. (daffodils), Petunia hybrida (petunias), Dianthus caryophyllus (carnation), Euphorbia pulcherrima (poinsettia), chrysanthemum, Nicotiana tabacum (tobacco), Lupinus albus (lupin), Uniola paniculata (oats), bentgrass (Agrostis spp.), Populus tremuloides (aspen), Pinus spp. (pine), Abies spp. (fir), Acer spp. (maple), Hordeum vulgare (barley), Poa pratensis (bluegrass), Lolium spp. (ryegrass), Phleum pratense (timothy), and conifers.

Conifers may be used in the present disclosure and include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Eastern or Canadian hemlock (Tsuga canadensis); Western hemlock (Tsuga heterophylla); Mountain hemlock (Tsuga mertensiana); Tamarack or Larch (Larix occidentalis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Turf grasses may be used in the present disclosure and include, but are not limited to: annual bluegrass (Poa annua); annual ryegrass (Lolium multiflorum); Canada bluegrass (Poa compressa); colonial bentgrass (Agrostis tenuis); creeping bentgrass (Agrostis palustris); crested wheatgrass (Agropyron desertorum); fairway wheatgrass (Agropyron cristatum); hard fescue (Festuca longifolia); Kentucky bluegrass (Poa pratensis); orchardgrass (Dactylis glomerata); perennial ryegrass (Lolium perenne); red fescue (Festuca rubra); redtop (Agrostis alba); rough bluegrass (Poa trivialis); sheep fescue (Festuca ovina); smooth bromegrass (Bromus inermis); timothy (Phleum pratense); velvet bentgrass (Agrostis canina); weeping alkaligrass (Puccinellia distans); western wheatgrass (Agropyron smithii); St. Augustine grass (Stenotaphrum secundatum); zoysia grass (Zoysia spp.); Bahia grass (Paspalum notatum); carpet grass (Axonopus affinis); centipede grass (Eremochloa ophiuroides); kikuyu grass (Pennisetum clandesinum); seashore paspalum (Paspalum vaginatum); blue gramma (Bouteloua gracilis); buffalo grass (Buchloe dactyloids); sideoats gramma (Bouteloua curtipendula).

In specific aspects, plants of the present disclosure are crop plants (for example, corn, alfalfa, sunflower, Brassica, soybean, cotton, safflower, peanut, rice. sorghum, wheat, millet, tobacco, etc.). Other plants useful in the present disclosure include barley, oats, rye, flax, sugarcane, banana, cassava, common bean, cowpea, tomato, potato, beet, grape, Eucalyptus, poplar, pine, douglas fir, citrus, papaya, cacao, cucumber, apple, Capsicum, bamboo, Triticale, and melon.

Additional heterologous coding sequences, heterologous polynucleotides, and polynucleotides of interest expressed by a promoter sequence of the disclosure may be used for varying the phenotype of a plant. Various changes in phenotype are of interest including modifying expression of a gene in a plant, altering a plant's pathogen or insect defense mechanism, increasing a plant's tolerance to herbicides, altering plant development to respond to environmental stress, modulating the plant's response to salt, temperature (hot and cold), drought and the like. These results can be achieved by the expression of a heterologous nucleotide sequence of interest comprising an appropriate gene product. In specific aspects, the heterologous nucleotide sequence of interest is an endogenous plant sequence whose expression level is increased in the plant or plant part. Results can be achieved by providing for altered expression of one or more endogenous gene products, particularly hormones, receptors, signaling molecules, enzymes, transporters or cofactors or by affecting nutrient uptake in the plant. Tissue-preferred expression as provided by the promoters disclosed herein can alter gene product expression. These changes result in a change in phenotype of the transformed plant. In certain aspects, since the expression pattern is tissue-preferred, the expression patterns are useful for many types of screening.

General categories of nucleotide sequences of interest for the present disclosure include, for example, those genes involved in information, such as zinc fingers, those involved in communication, such as kinases and those involved in housekeeping, such as heat shock proteins. More specific categories of transgenes, for example, include genes encoding important traits for agronomics, insect resistance, disease resistance, herbicide resistance, environmental stress resistance (altered tolerance to cold, salt, drought, etc.) and grain characteristics. Still other categories of transgenes include genes for inducing expression of exogenous products such as enzymes, cofactors, and hormones from plants and other eukaryotes as well as prokaryotic organisms. It is recognized that any gene or polynucleotide of interest can be operably linked to a promoter of the disclosure and expressed in a plant.

Multiple genes of interest can be operably linked to a promoter of the disclosure and expressed in a plant, for example the WUS/WOX genes can be stacked with insect resistance traits which can also be stacked with one or more additional input traits (e.g., herbicide resistance, fungal resistance, virus resistance, stress tolerance, disease resistance, male sterility, stalk strength, and the like) or output traits (e.g., increased yield, modified starches, improved oil profile, balanced amino acids, high lysine or methionine, increased digestibility, improved fiber quality, drought resistance, nutritional enhancement, and the like).

A promoter of the disclosure can be operably linked to agronomically important traits that affect quality of grain, such as levels (increasing content of oleic acid) and types of oils, saturated and unsaturated, quality and quantity of essential amino acids, increasing levels of lysine and sulfur, levels of cellulose, and starch and protein content. A promoter of the disclosure can be operably linked to genes providing hordothionin protein modifications in corn which are described in U.S. Pat. Nos. 5,990,389; 5,885,801; 5,885,802 and 5,703,049; herein incorporated by reference in their entirety. Another example of a gene to which a promoter of the disclosure can be operably linked to is a lysine and/or sulfur rich seed protein encoded by the soybean 2S albumin described in U.S. Pat. No. 5,850,016, filed Mar. 20, 1996 and the chymotrypsin inhibitor from barley, Williamson, et al., (1987) Eur. J. Biochem 165:99-106, the disclosures of which are herein incorporated by reference in their entirety.

A promoter of the disclosure can be operably linked to insect resistance genes that encode resistance to pests that have great yield drag such as rootworm, cutworm, European corn borer and the like. Such genes include, for example, Bacillus thuringiensis toxic protein genes, U.S. Pat. Nos. 5,366,892; 5,747,450; 5,736,514; 5,723,756; 5,593,881 and Geiser, et al., (1986) Gene 48:109, the disclosures of which are herein incorporated by reference in their entirety. Genes encoding disease resistance traits that can be operably linked to a promoter of the disclosure include, for example, detoxification genes, such as those which detoxify fumonisin (U.S. Pat. No. 5,792,931); avirulence (avr) and disease resistance (R) genes (Jones, et al., (1994) Science 266:789; Martin, et al., (1993) Science 262:1432; and Mindrinos, et al., (1994) Cell 78:1089), herein incorporated by reference in their entirety.

Herbicide resistance traits that can be operably linked to a promoter of the disclosure include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), genes coding for resistance to glyphosate (e.g., the EPSPS gene and the GAT gene; see, for example, US Patent Application Publication Number 2004/0082770 and WO 03/092360, herein incorporated by reference in their entirety) or other such genes known in the art. The bar gene encodes resistance to the herbicide basta, the nptII gene encodes resistance to the antibiotics kanamycin and geneticin and the ALS-gene mutants encode resistance to the herbicide chlorsulfuron any and all of which can be operably linked to a promoter of the disclosure.

Glyphosate resistance is imparted by mutant 5-enolpyruvl-3-phosphikimate synthase (EPSP) and aroA genes which can be operably linked to a promoter of the disclosure. See, for example, U.S. Pat. No. 4,940,835 to Shah, et al., which discloses the nucleotide sequence of a form of EPSPS which can confer glyphosate resistance. U.S. Pat. No. 5,627,061 to Barry, et al., also describes genes encoding EPSPS enzymes which can be operably linked to a promoter of the disclosure. See also, U.S. Pat. Nos. 6,248,876 B1; 6,040,497; 5,804,425; 5,633,435; 5,145,783; 4,971,908; 5,312,910; 5,188,642; 4,940,835; 5,866,775; 6,225,114 B1; 6,130,366; 5,310,667; 4,535,060; 4,769,061; 5,633,448; 5,510,471; Re. 36,449; RE 37,287 E and 5,491,288 and international publications WO 97/04103; WO 97/04114; WO 00/66746; WO 01/66704; WO 00/66747 and WO 00/66748, which are incorporated herein by reference in their entirety. Glyphosate resistance is also imparted to plants that express a gene which can be operably linked to a promoter of the disclosure that encodes a glyphosate oxido-reductase enzyme as described more fully in U.S. Pat. Nos. 5,776,760 and 5,463,175, which are incorporated herein by reference in their entirety. Glyphosate resistance can also be imparted to plants by the over expression of genes which can be operably linked to a promoter of the disclosure encoding glyphosate N-acetyltransferase. See, for example, U.S. patent application Ser. Nos. 11/405,845 and 10/427,692, herein incorporated by reference in their entirety.

Sterility genes operably linked to a promoter of the disclosure can also be encoded in a DNA construct and provide an alternative to physical detasseling. Examples of genes used in such ways include male tissue-preferred genes and genes with male sterility phenotypes such as QM, described in U.S. Pat. No. 5,583,210, herein incorporated by reference in its entirety. Other genes which can be operably linked to a promoter of the disclosure include kinases and those encoding compounds toxic to either male or female gametophytic development.

Commercial traits can also be encoded on a gene or genes operably linked to a promoter of the disclosure that could increase for example, starch for ethanol production, or provide expression of proteins. Another important commercial use of transformed plants is the production of polymers and bioplastics such as described in U.S. Pat. No. 5,602,321, herein incorporated by reference in its entirety. Genes such as β-Ketothiolase, PHBase (polyhydroxybutyrate synthase), and acetoacetyl-CoA reductase which can be operably linked to a promoter of the disclosure (see, Schubert, et al., (1988) J. Bacteriol. 170:5837-5847, herein incorporated by reference in its entirety) facilitate expression of polyhydroxyalkanoates (PHAs).

Examples of other applicable genes and their associated phenotype which can be operably linked to a promoter of the disclosure include the gene which encodes viral coat protein and/or RNA, or other viral or plant genes that confer viral resistance; genes that confer fungal resistance; genes that promote yield improvement; and genes that provide for resistance to stress, such as cold, dehydration resulting from drought, heat and salinity, toxic metal or trace elements or the like.

By way of illustration, without intending to be limiting, the following is a list of other examples of the types of genes which can be operably linked to a promoter sequence of the disclosure.

1. Transgenes That Confer Resistance To Insects Or Disease And That Encode:

(A) Plant disease resistance genes. Plant defenses are often activated by specific interaction between the product of a disease resistance gene (R) in the plant and the product of a corresponding avirulence (Avr) gene in the pathogen. A plant variety can be transformed with a cloned resistance gene to engineer plants that are resistant to specific pathogen strains. See, for example Jones, et al., (1994) Science 266:789 (cloning of the tomato Cf-9 gene for resistance to Cladosporium fulvum); Martin, et al., (1993) Science 262:1432 (tomato Pto gene for resistance to Pseudomonas syringae pv. tomato encodes a protein kinase); Mindrinos, et al., (1994) Cell 78:1089 (Arabidopsis RSP2 gene for resistance to Pseudomonas syringae); McDowell and Woffenden, (2003) Trends Biotechnol. 21(4):178-83 and Toyoda, et al., (2002) Transgenic Res. 11(6):567-82, herein incorporated by reference in their entirety. A plant resistant to a disease is one that is more resistant to a pathogen as compared to the wild type plant.

(B) A Bacillus thuringiensis protein, a derivative thereof or a synthetic polypeptide modeled thereon. See, for example, Geiser, et al., (1986) Gene 48:109, who disclose the cloning and nucleotide sequence of a Bt delta-endotoxin gene. Moreover, DNA molecules encoding delta-endotoxin genes can be purchased from American Type Culture Collection (Rockville, Md.), for example, under ATCC Accession Numbers 40098, 67136, 31995 and 31998. Other examples of Bacillus thuringiensis transgenes being genetically engineered are given in the following patents and patent applications and hereby are incorporated by reference for this purpose: U.S. Pat. Nos. 5,188,960; 5,689,052; 5,880,275; WO 91/14778; WO 99/31248; WO 01/12731; WO 99/24581; WO 97/40162 and U.S. application Ser. Nos. 10/032,717; 10/414,637 and 10/606,320, herein incorporated by reference in their entirety.

(C) An insect-specific hormone or pheromone such as an ecdysteroid and juvenile hormone, a variant thereof, a mimetic based thereon, or an antagonist or agonist thereof. See, for example, the disclosure by Hammock, et al., (1990) Nature 344:458, of baculovirus expression of cloned juvenile hormone esterase, an inactivator of juvenile hormone, herein incorporated by reference in its entirety.

(D) An insect-specific peptide which, upon expression, disrupts the physiology of the affected pest. For example, see the disclosures of Regan, (1994) J. Biol. Chem. 269:9 (expression cloning yields DNA coding for insect diuretic hormone receptor); Pratt, et al., (1989) Biochem. Biophys. Res. Comm. 163:1243 (an allostatin is identified in Diploptera puntata); Chattopadhyay, et al., (2004) Critical Reviews in Microbiology 30(1):33-54; Zjawiony, (2004) J Nat Prod 67(2):300-310; Carlini and Grossi-de-Sa, (2002) Toxicon 40(11):1515-1539; Ussuf, et al., (2001) Curr Sci. 80(7):847-853 and Vasconcelos and Oliveira, (2004) Toxicon 44(4):385-403, herein incorporated by reference in their entirety. See also, U.S. Pat. No. 5,266,317 to Tomalski, et al., who disclose genes encoding insect-specific toxins, herein incorporated by reference in its entirety.

(E) An enzyme responsible for a hyperaccumulation of a monterpene, a sesquiterpene, a steroid, hydroxamic acid, a phenylpropanoid derivative or another non-protein molecule with insecticidal activity.

(F) An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See, PCT Application Number WO 93/02197 in the name of Scott, et al., which discloses the nucleotide sequence of a callase gene, herein incorporated by reference in its entirety. DNA molecules which contain chitinase-encoding sequences can be obtained, for example, from the ATCC under Accession Numbers 39637 and 67152. See also, Kramer, et al., (1993) Insect Biochem. Molec. Biol. 23:691, who teach the nucleotide sequence of a cDNA encoding tobacco hookworm chitinase, and Kawalleck, et al., (1993) Plant Molec. Biol. 21:673, who provide the nucleotide sequence of the parsley ub14-2 polyubiquitin gene, U.S. patent application Ser. Nos. 10/389,432, 10/692,367 and U.S. Pat. No. 6,563,020, herein incorporated by reference in their entirety.

(G) A molecule that stimulates signal transduction. For example, see the disclosure by Botella, et al., (1994) Plant Molec. Biol. 24:757, of nucleotide sequences for mung bean calmodulin cDNA clones, and Griess, et al., (1994) Plant Physiol. 104:1467, who provide the nucleotide sequence of a maize calmodulin cDNA clone, herein incorporated by reference in their entirety.

(H) A hydrophobic moment peptide. See, PCT Application Number WO 95/16776 and U.S. Pat. No. 5,580,852 (disclosure of peptide derivatives of Tachyplesin which inhibit fungal plant pathogens) and PCT Application Number WO 95/18855 and U.S. Pat. No. 5,607,914) (teaches synthetic antimicrobial peptides that confer disease resistance), herein incorporated by reference in their entirety.

(I) A membrane permease, a channel former or a channel blocker. For example, see the disclosure by Jaynes, et al., (1993) Plant Sci. 89:43, of heterologous expression of a cecropin-beta lytic peptide analog to render transgenic tobacco plants resistant to Pseudomonas solanacearum, herein incorporated by reference in its entirety.

(J) A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See, Beachy, et al., (1990) Ann. Rev. Phytopathol. 28:451, herein incorporated by reference in its entirety. Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.

(K) An insect-specific antibody or an immunotoxin derived therefrom. Thus, an antibody targeted to a critical metabolic function in the insect gut would inactivate an affected enzyme, killing the insect. Cf Taylor, et al., Abstract #497, SEVENTH INT'L SYMPOSIUM ON MOLECULAR PLANT-MICROBE INTERACTIONS (Edinburgh, Scotland, 1994) (enzymatic inactivation in transgenic tobacco via production of single-chain antibody fragments), herein incorporated by reference in its entirety.

(L) A virus-specific antibody. See, for example, Tavladoraki, et al., (1993) Nature 366:469, who show that transgenic plants expressing recombinant antibody genes are protected from virus attack, herein incorporated by reference in its entirety.

(M) A developmental-arrestive protein produced in nature by a pathogen or a parasite. Thus, fungal endo alpha-1,4-D-polygalacturonases facilitate fungal colonization and plant nutrient release by solubilizing plant cell wall homo-alpha-1,4-D-galacturonase. See, Lamb, et al., (1992) Bio/Technology 10:1436, herein incorporated by reference in its entirety. The cloning and characterization of a gene which encodes a bean endopolygalacturonase-inhibiting protein is described by Toubart, et al., (1992) Plant J. 2:367, herein incorporated by reference in its entirety.

(N) A developmental-arrestive protein produced in nature by a plant. For example, Logemann, et al., (1992) Bio/Technology 10:305, herein incorporated by reference in its entirety, have shown that transgenic plants expressing the barley ribosome-inactivating gene have an increased resistance to fungal disease.

(O) Genes involved in the Systemic Acquired Resistance (SAR) Response and/or the pathogenesis related genes. Briggs, (1995) Current Biology 5(2):128-131, Pieterse and Van Loon, (2004) Curr. Opin. Plant Bio. 7(4):456-64 and Somssich, (2003) Cell 113(7):815-6, herein incorporated by reference in their entirety.

(P) Antifungal genes (Cornelissen and Melchers, (1993) Pl. Physiol. 101:709-712 and Parijs, et al., (1991) Planta 183:258-264 and Bushnell, et al., (1998) Can. J. of Plant Path. 20(2):137-149. Also see, U.S. patent application Ser. No. 09/950,933, herein incorporated by reference in their entirety.

(Q) Detoxification genes, such as for fumonisin, beauvericin, moniliformin and zearalenone and their structurally related derivatives. For example, see, U.S. Pat. No. 5,792,931, herein incorporated by reference in its entirety.

(R) Cystatin and cysteine proteinase inhibitors. See, U.S. application Ser. No. 10/947,979, herein incorporated by reference in its entirety.

(S) Defensin genes. See, WO03/000863 and U.S. application Ser. No. 10/178,213, herein incorporated by reference in their entirety.

(T) Genes conferring resistance to nematodes. See, WO 03/033651 and Urwin, et. al., (1998) Planta 204:472-479, Williamson (1999) Curr Opin Plant Bio. 2(4):327-31, herein incorporated by reference in their entirety.

(U) Genes such as rcglconferring resistance to Anthracnose stalk rot, which is caused by the fungus Colletotrichum graminiola. See, Jung, et al., Generation-means analysis and quantitative trait locus mapping of Anthracnose Stalk Rot genes in Maize, Theor. Appl. Genet. (1994) 89:413-418, as well as, US Provisional Patent Application Number 60/675,664, herein incorporated by reference in their entirety.

2. Transgenes That Confer Resistance To A Herbicide, For Example:

(A) A herbicide that inhibits the growing point or meristem, such as an imidazolinone or a sulfonylurea. Exemplary genes in this category code for mutant ALS and AHAS enzyme as described, for example, by Lee, et al., (1988) EMBO J. 7:1241 and Miki, et al., (1990) Theor. Appl. Genet. 80:449, respectively. See also, U.S. Pat. Nos. 5,605,011; 5,013,659; 5,141,870; 5,767,361; 5,731,180; 5,304,732; 4,761,373; 5,331,107; 5,928,937 and 5,378,824 and international publication WO 96/33270, which are incorporated herein by reference in their entirety.

(B) Glyphosate (resistance imparted by mutant 5-enolpyruvl-3-phosphikimate synthase (EPSP) and aroA genes, respectively) and other phosphono compounds such as glufosinate (phosphinothricin acetyl transferase (PAT) and Streptomyces hygroscopicus phosphinothricin acetyl transferase (bar) genes) and pyridinoxy or phenoxy proprionic acids and cycloshexones (ACCase inhibitor-encoding genes). See, for example, U.S. Pat. No. 4,940,835 to Shah, et al., which discloses the nucleotide sequence of a form of EPSPS which can confer glyphosate resistance. U.S. Pat. No. 5,627,061 to Barry, et al., also describes genes encoding EPSPS enzymes. See also, U.S. Pat. Nos. 6,566,587; 6,338,961; 6,248,876 B1; 6,040,497; 5,804,425; 5,633,435; 5,145,783; 4,971,908; 5,312,910; 5,188,642; 4,940,835; 5,866,775; 6,225,114 B1; 6,130,366; 5,310,667; 4,535,060; 4,769,061; 5,633,448; 5,510,471; Re. 36,449; RE 37,287 E and 5,491,288 and international publications EP1173580; WO 01/66704; EP1173581 and EP1173582, which are incorporated herein by reference in their entirety. Glyphosate resistance is also imparted to plants that express a gene that encodes a glyphosate oxido-reductase enzyme as described more fully in U.S. Pat. Nos. 5,776,760 and 5,463,175, which are incorporated herein by reference in their entirety. In addition glyphosate resistance can be imparted to plants by the over expression of genes encoding glyphosate N-acetyltransferase. See, for example, U.S. patent application Ser. Nos. 11/405,845 and 10/427,692 and PCT Application Number US01/46227, herein incorporated by reference in their entirety. A DNA molecule encoding a mutant aroA gene can be obtained under ATCC Accession Number 39256 and the nucleotide sequence of the mutant gene is disclosed in U.S. Pat. No. 4,769,061 to Comai, herein incorporated by reference in its entirety. EP Patent Application Number 0 333 033 to Kumada, et al., and U.S. Pat. No. 4,975,374 to Goodman, et al., disclose nucleotide sequences of glutamine synthetase genes which confer resistance to herbicides such as L-phosphinothricin, herein incorporated by reference in their entirety. The nucleotide sequence of a phosphinothricin-acetyl-transferase gene is provided in EP Patent Numbers 0 242 246 and 0 242 236 to Leemans, et al., De Greef, et al., (1989) Bio/Technology 7:61 which describe the production of transgenic plants that express chimeric bar genes coding for phosphinothricin acetyl transferase activity, herein incorporated by reference in their entirety. See also, U.S. Pat. Nos. 5,969,213; 5,489,520; 5,550,318; 5,874,265; 5,919,675; 5,561,236; 5,648,477; 5,646,024; 6,177,616 B1 and 5,879,903, herein incorporated by reference in their entirety. Exemplary genes conferring resistance to phenoxy proprionic acids and cycloshexones, such as sethoxydim and haloxyfop, are the Acc1-S1, Acc1-S2 and Acc1-S3 genes described by Marshall, et al., (1992) Theor. Appl. Genet. 83:435, herein incorporated by reference in its entirety.

(C) A herbicide that inhibits photosynthesis, such as a triazine (psbA and gs+ genes) and a benzonitrile (nitrilase gene). Przibilla, et al., (1991) Plant Cell 3:169, herein incorporated by reference in its entirety, describe the transformation of Chlamydomonas with plasmids encoding mutant psbA genes. Nucleotide sequences for nitrilase genes are disclosed in U.S. Pat. No. 4,810,648 to Stalker, herein incorporated by reference in its entirety, and DNA molecules containing these genes are available under ATCC Accession Numbers 53435, 67441 and 67442. Cloning and expression of DNA coding for a glutathione S-transferase is described by Hayes, et al., (1992) Biochem. 1 285:173, herein incorporated by reference in its entirety.

(D) Acetohydroxy acid synthase, which has been found to make plants that express this enzyme resistant to multiple types of herbicides, has been introduced into a variety of plants (see, e.g., Hattori, et al., (1995) Mol Gen Genet 246:419, herein incorporated by reference in its entirety). Other genes that confer resistance to herbicides include: a gene encoding a chimeric protein of rat cytochrome P4507A1 and yeast NADPH-cytochrome P450 oxidoreductase (Shiota, et al., (1994) Plant Physiol. 106(1):17-23), genes for glutathione reductase and superoxide dismutase (Aono, et al., (1995) Plant Cell Physiol 36:1687, and genes for various phosphotransferases (Datta, et al., (1992) Plant Mol Biol 20:619), herein incorporated by reference in their entirety.

(E) Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306 B1; 6,282,837 B1 and 5,767,373; and international publication number WO 01/12825, herein incorporated by reference in their entirety.

3. Transgenes That Confer Or Contribute To an Altered Grain Characteristic, Such As:

(A) Altered fatty acids, for example, by

(1) Down-regulation of stearoyl-ACP desaturase to increase stearic acid content of the plant. See, Knultzon, et al., (1992) Proc. Natl. Acad. Sci. USA 89:2624 and WO99/64579 (Genes for Desaturases to Alter Lipid Profiles in Corn), herein incorporated by reference in their entirety,

(2) Elevating oleic acid via FAD-2 gene modification and/or decreasing linolenic acid via FAD-3 gene modification (see, U.S. Pat. Nos. 6,063,947; 6,323,392; 6,372,965 and WO 93/11245, herein incorporated by reference in their entirety),

(3) Altering conjugated linolenic or linoleic acid content, such as in WO 01/12800, herein incorporated by reference in its entirety,

(4) Altering LEC1, AGP, Dek1, Superal1, mi1ps, various 1pa genes such as 1pa1, 1pa3, hpt or hggt. For example, see, WO 02/42424, WO 98/22604, WO 03/011015, U.S. Pat. Nos. 6,423,886, 6,197,561, 6,825,397, US Patent Application Publication Numbers 2003/0079247, 2003/0204870, WO02/057439, WO03/011015 and Rivera-Madrid, et. al., (1995) Proc. Natl. Acad. Sci. 92:5620-5624, herein incorporated by reference in their entirety.

(B) Altered phosphorus content, for example, by the

(1) Introduction of a phytase-encoding gene would enhance breakdown of phytate, adding more free phosphate to the transformed plant. For example, see, Van Hartingsveldt, et al., (1993) Gene 127:87, for a disclosure of the nucleotide sequence of an Aspergillus niger phytase gene, herein incorporated by reference in its entirety.

(2) Up-regulation of a gene that reduces phytate content. In maize, this, for example, could be accomplished, by cloning and then re-introducing DNA associated with one or more of the alleles, such as the LPA alleles, identified in maize mutants characterized by low levels of phytic acid, such as in Raboy, et al., (1990) Maydica 35:383 and/or by altering inositol kinase activity as in WO 02/059324, US Patent Application Publication Number 2003/0009011, WO 03/027243, US Patent Application Publication Number 2003/0079247, WO 99/05298, U.S. Pat. Nos. 6,197,561, 6,291,224, 6,391,348, WO2002/059324, US Patent Application Publication Number 2003/0079247, WO98/45448, WO99/55882, WO01/04147, herein incorporated by reference in their entirety.

(C) Altered carbohydrates effected, for example, by altering a gene for an enzyme that affects the branching pattern of starch or a gene altering thioredoxin such as NTR and/or TRX (see, U.S. Pat. No. 6,531,648, which is incorporated by reference in its entirety) and/or a gamma zein knock out or mutant such as cs27 or TUSC27 or en27 (see, U.S. Pat. No. 6,858,778 and US Patent Application Publication Numbers 2005/0160488 and 2005/0204418; which are incorporated by reference in its entirety). See, Shiroza, et al., (1988) J. Bacteriol. 170:810 (nucleotide sequence of Streptococcus mutans fructosyltransferase gene), Steinmetz, et al., (1985) Mol. Gen. Genet. 200:220 (nucleotide sequence of Bacillus subtilis levansucrase gene), Pen, et al., (1992) Bio/Technology 10:292 (production of transgenic plants that express Bacillus licheniformis alpha-amylase), Elliot, et al., (1993) Plant Molec. Biol. 21:515 (nucleotide sequences of tomato invertase genes), Søgaard, et al., (1993) J. Biol. Chem. 268:22480 (site-directed mutagenesis of barley alpha-amylase gene) and Fisher, et al., (1993) Plant Physiol. 102:1045 (maize endosperm starch branching enzyme II), WO 99/10498 (improved digestibility and/or starch extraction through modification of UDP-D-xylose 4-epimerase, Fragile 1 and 2, Ref1, HCHL, C4H), U.S. Pat. No. 6,232,529 (method of producing high oil seed by modification of starch levels (AGP)), herein incorporated by reference in their entirety. The fatty acid modification genes mentioned above may also be used to affect starch content and/or composition through the interrelationship of the starch and oil pathways.

(D) Altered antioxidant content or composition, such as alteration of tocopherol or tocotrienols. For example, see U.S. Pat. No. 6,787,683, US Patent Application Publication Number 2004/0034886 and WO 00/68393 involving the manipulation of antioxidant levels through alteration of a phytl prenyl transferase (ppt), WO 03/082899 through alteration of a homogentisate geranyl geranyl transferase (hggt), herein incorporated by reference in their entirety.

(E) Altered essential seed amino acids. For example, see U.S. Pat. No. 6,127,600 (method of increasing accumulation of essential amino acids in seeds), U.S. Pat. No. 6,080,913 (binary methods of increasing accumulation of essential amino acids in seeds), U.S. Pat. No. 5,990,389 (high lysine), WO99/40209 (alteration of amino acid compositions in seeds), WO99/29882 (methods for altering amino acid content of proteins), U.S. Pat. No. 5,850,016 (alteration of amino acid compositions in seeds), WO98/20133 (proteins with enhanced levels of essential amino acids), U.S. Pat. No. 5,885,802 (high methionine), U.S. Pat. No. 5,885,801 (high threonine), U.S. Pat. No. 6,664,445 (plant amino acid biosynthetic enzymes), U.S. Pat. No. 6,459,019 (increased lysine and threonine), U.S. Pat. No. 6,441,274 (plant tryptophan synthase beta subunit), U.S. Pat. No. 6,346,403 (methionine metabolic enzymes), U.S. Pat. No. 5,939,599 (high sulfur), U.S. Pat. No. 5,912,414 (increased methionine), WO98/56935 (plant amino acid biosynthetic enzymes), WO98/45458 (engineered seed protein having higher percentage of essential amino acids), WO98/42831 (increased lysine), U.S. Pat. No. 5,633,436 (increasing sulfur amino acid content), U.S. Pat. No. 5,559,223 (synthetic storage proteins with defined structure containing programmable levels of essential amino acids for improvement of the nutritional value of plants), WO96/01905 (increased threonine), WO95/15392 (increased lysine), US Patent Application Publication Number 2003/0163838, US Patent Application Publication Number 2003/0150014, US Patent Application Publication Number 2004/0068767, U.S. Pat. No. 6,803,498, WO01/79516, and WO00/09706 (Ces A: cellulose synthase), U.S. Pat. No. 6,194,638 (hemicellulose), U.S. Pat. No. 6,399,859 and US Patent Application Publication Number 2004/0025203 (UDPGdH), U.S. Pat. No. 6,194,638 (RGP), herein incorporated by reference in their entirety.

4. Genes that create a site for site specific DNA integration

This includes the introduction of FRT sites that may be used in the FLP/FRT system and/or Lox sites that may be used in the Cre/Loxp system. For example, see Lyznik, et al., (2003) Plant Cell Rep 21:925-932 and WO 99/25821, which are hereby incorporated by reference in their entirety. Other systems that may be used include the Gin recombinase of phage Mu (Maeser, et al., 1991; Vicki Chandler, The Maize Handbook ch. 118 (Springer-Verlag 1994), the Pin recombinase of E. coli (Enomoto, et al., 1983), and the R/RS system of the pSR1 plasmid (Araki, et al., 1992), herein incorporated by reference in their entirety.

5. Genes that affect abiotic stress resistance (including but not limited to flowering, ear and seed development, enhancement of nitrogen utilization efficiency, altered nitrogen responsiveness, drought resistance or tolerance, cold resistance or tolerance, and salt resistance or tolerance) and increased yield under stress. For example, see, WO 00/73475 where water use efficiency is altered through alteration of malate; U.S. Pat. Nos. 5,892,009, 5,965,705, 5,929,305, 5,891,859, 6,417,428, 6,664,446, 6,706,866, 6,717,034, WO2000060089, WO2001026459, WO2001035725, WO2001034726, WO2001035727, WO2001036444, WO2001036597, WO2001036598, WO2002015675, WO2002017430, WO2002077185, WO2002079403, WO2003013227, WO2003013228, WO2003014327, WO2004031349, WO2004076638, WO9809521, and WO9938977 describing genes, including CBF genes and transcription factors effective in mitigating the negative effects of freezing, high salinity, and drought on plants, as well as conferring other positive effects on plant phenotype; US Patent Application Publication Number 2004/0148654 and WO01/36596 where abscisic acid is altered in plants resulting in improved plant phenotype such as increased yield and/or increased tolerance to abiotic stress; WO2000/006341, WO04/090143, U.S. patent application Ser. No. 10/817,483 and U.S. Pat. No. 6,992,237, where cytokinin expression is modified resulting in plants with increased stress tolerance, such as drought tolerance, and/or increased yield, herein incorporated by reference in their entirety. Also see WO0202776, WO2003052063, JP2002281975, U.S. Pat. No. 6,084,153, WO0164898, U.S. Pat. Nos. 6,177,275 and 6,107,547 (enhancement of nitrogen utilization and altered nitrogen responsiveness), herein incorporated by reference in their entirety. For ethylene alteration, see US Patent Application Publication Number 2004/0128719, US Patent Application Publication Number 2003/0166197 and WO200032761, herein incorporated by reference in their entirety. For plant transcription factors or transcriptional regulators of abiotic stress, see, e.g., US Patent Application Publication Number 2004/0098764 or US Patent Application Publication Number 2004/0078852, herein incorporated by reference in their entirety.

6. Other genes and transcription factors that affect plant growth and agronomic traits such as yield, flowering, plant growth and/or plant structure, can be introduced or introgressed into plants, see, e.g., WO97/49811 (LHY), WO98/56918 (ESD4), WO97/10339 and U.S. Pat. No. 6,573,430 (TFL), U.S. Pat. No. 6,713,663 (FT), WO96/14414 (CON), WO96/38560, WO01/21822 (VRN1), WO00/44918 (VRN2), WO99/49064 (GI), WO00/46358 (FRI), WO97/29123, U.S. Pat. Nos. 6,794,560, 6,307,126 (GAI), WO99/09174 (D8 and Rht) and WO2004076638 and WO2004031349 (transcription factors), herein incorporated by reference in their entirety.

The heterologous nucleotide sequence operably linked to a promoter sequence and its related biologically active fragments or variants disclosed herein may be an antisense sequence for a targeted gene. The terminology “antisense DNA nucleotide sequence” is intended to mean a sequence that is in inverse orientation to the 5′-to-3′ normal orientation of that nucleotide sequence. When delivered into a plant cell, expression of the antisense DNA sequence prevents normal expression of the DNA nucleotide sequence for the targeted gene. The antisense nucleotide sequence encodes an RNA transcript that is complementary to and capable of hybridizing to the endogenous messenger RNA (mRNA) produced by transcription of the DNA nucleotide sequence for the targeted gene. In this case, production of the native protein encoded by the targeted gene is inhibited to achieve a desired phenotypic response. Modifications of the antisense sequences may be made as long as the sequences hybridize to and interfere with expression of the corresponding mRNA. In this manner, antisense constructions having 70%, 80%, 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides or greater may be used. Thus, the promoter sequences disclosed herein may be operably linked to antisense DNA sequences to reduce or inhibit expression of a native protein in the plant.

“RNAi” refers to a series of related techniques to reduce the expression of genes (see, for example, U.S. Pat. No. 6,506,559, herein incorporated by reference in its entirety). Older techniques referred to by other names are now thought to rely on the same mechanism, but are given different names in the literature. These include “antisense inhibition,” the production of antisense RNA transcripts capable of suppressing the expression of the target protein and “co-suppression” or “sense-suppression,” which refer to the production of sense RNA transcripts capable of suppressing the expression of identical or substantially similar foreign or endogenous genes (U.S. Pat. No. 5,231,020, incorporated herein by reference in its entirety). Such techniques rely on the use of constructs resulting in the accumulation of double stranded RNA with one strand complementary to the target gene to be silenced. The promoter sequences of the disclosure may be used to drive expression of constructs that will result in RNA interference including microRNAs and siRNAs.

As used herein, the terms “promoter” or “transcriptional initiation region” mean a regulatory region of DNA usually comprising a TATA box or a DNA sequence capable of directing RNA polymerase II to initiate RNA synthesis at the appropriate transcription initiation site for a particular coding sequence. A promoter may additionally comprise other recognition sequences generally positioned upstream or 5′ to the TATA box or the DNA sequence capable of directing RNA polymerase II to initiate RNA synthesis, referred to as upstream promoter elements, which influence the transcription initiation rate. It is recognized that having identified the nucleotide sequences for the promoter regions disclosed herein, it is within the state of the art to isolate and identify further promoters in the 5′ untranslated region upstream from the particular promoter regions identified herein. Additionally, chimeric promoters may be provided. Such chimeras include portions of the promoter sequence fused to fragments and/or variants of heterologous transcriptional regulatory regions. Thus, the promoter regions disclosed herein can comprise upstream promoters such as, those responsible for tissue and temporal expression of the coding sequence, enhancers and the like.

As used herein, the term “regulatory element” also refers to a sequence of DNA, usually, but not always, upstream (5′) to the coding sequence of a structural gene, which includes sequences which control the expression of the coding region by providing the recognition for RNA polymerase and/or other factors required for transcription to start at a particular site. An example of a regulatory element that provides for the recognition for RNA polymerase or other transcriptional factors to ensure initiation at a particular site is a promoter element. A promoter element comprises a core promoter element, responsible for the initiation of transcription, as well as other regulatory elements that modify gene expression. It is to be understood that nucleotide sequences, located within introns or 3′ of the coding region sequence may also contribute to the regulation of expression of a coding region of interest. Examples of suitable introns include, but are not limited to, the maize IVS6 intron, or the maize actin intron. A regulatory element may also include those elements located downstream (3′) to the site of transcription initiation, or within transcribed regions, or both. In the context of the present disclosure a post-transcriptional regulatory element may include elements that are active following transcription initiation, for example translational and transcriptional enhancers, translational and transcriptional repressors and mRNA stability determinants.

The promoters or variants or fragments thereof of the present disclosure, may be operatively associated with heterologous regulatory elements or promoters to modulate the activity of the heterologous regulatory element. Such modulation includes enhancing or repressing transcriptional activity of the heterologous regulatory element, modulating post-transcriptional events, or either enhancing or repressing transcriptional activity of the heterologous regulatory element and modulating post-transcriptional events. For example, one or more promoters or fragments thereof of the present disclosure may be operatively associated with constitutive, inducible or tissue specific promoters or fragments thereof, to modulate the activity of such promoters within desired tissues in plant cells.

The promoter sequences of the present disclosure or variants or fragments thereof, when operably linked to a morphogenic gene and/or heterologous nucleotide sequence of interest can drive stable or transient expression of the morphogenic gene and/or heterologous nucleotide sequence in the L1 tissue of the plant.

A “heterologous nucleotide sequence”, “heterologous polynucleotide of interest”, or “heterologous polynucleotide” as used throughout the disclosure, is a sequence that is not naturally occurring with or operably linked to a promoter sequence of the disclosure. While this nucleotide sequence is heterologous to the promoter sequence, it may be homologous or native or heterologous or foreign to the plant host. Likewise, the promoter sequence may be homologous or native or heterologous or foreign to the plant host and/or the polynucleotide of interest.

The isolated promoter sequences of the present disclosure can be modified to provide for a range of expression levels of the heterologous nucleotide sequence. Thus, less than the entire promoter region may be utilized and the ability to drive expression of the nucleotide sequence of interest retained. It is recognized that expression levels of the mRNA may be altered in different ways with deletions of portions of the promoter sequences. The mRNA expression levels may be decreased, or alternatively, expression may be increased as a result of promoter deletions if, for example, there is a negative regulatory element (for a repressor) that is removed during the truncation process. Generally, at least about 20 nucleotides of an isolated promoter sequence will be used to drive expression of a nucleotide sequence.

It is recognized that to increase transcription levels, enhancers may be utilized in combination with the promoter regions of the disclosure. Enhancers are nucleotide sequences that act to increase the expression of a promoter region. Enhancers are known in the art and include the SV40 enhancer region, the 35S enhancer element and the like. Some enhancers are also known to alter normal promoter expression patterns, for example, by causing a promoter to be expressed constitutively when without the enhancer, the same promoter is expressed only in one specific tissue or a few specific tissues.

Modifications of the isolated promoter sequences of the present disclosure can provide for a range of expression of the heterologous nucleotide sequence. Thus, they may be modified to be weak promoters or strong promoters. Generally, a “weak promoter” means a promoter that drives expression of a coding sequence at a low level. A “low level” of expression is intended to mean expression at levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts.

It is recognized that the promoters of the disclosure may be used with their native coding sequences to increase or decrease expression, thereby resulting in a change in phenotype of the transformed plant. The nucleotide sequences disclosed herein (see Table 1), as well as variants and fragments thereof, are useful in the genetic manipulation of any plant. The regulatory sequences are useful in this aspect when operably linked with a heterologous nucleotide sequence whose expression is to be controlled to achieve a desired phenotypic response. The term “operably linked” means that the transcription or translation of the heterologous nucleotide sequence is under the influence of the promoter sequence. In this manner, the nucleotide sequences for the promoters of the disclosure may be provided in expression cassettes along with heterologous nucleotide sequences of interest for expression in the plant of interest, more particularly for expression in the reproductive tissue of the plant.

In one aspect of the disclosure, expression cassettes comprise a transcriptional initiation region comprising one of the promoter nucleotide sequences of the present disclosure or variants or fragments thereof, operably linked to a morphogenic gene and/or a heterologous nucleotide sequence. Such an expression cassette can be provided with a plurality of restriction sites for insertion of the nucleotide sequence to be under the transcriptional regulation of the regulatory regions. The expression cassette may additionally contain selectable marker genes as well as 3′ termination regions.

The expression cassette can include, in the 5′-3′ direction of transcription, a transcriptional initiation region (i.e., a promoter, or variant or fragment thereof, of the disclosure), a translational initiation region, a heterologous nucleotide sequence of interest, a translational termination region and optionally, a transcriptional termination region functional in the host organism. The regulatory regions (i.e., promoters, transcriptional regulatory regions, and translational termination regions) and/or the polynucleotide of the aspects may be native/analogous to the host cell or to each other. Alternatively, the regulatory regions and/or the polynucleotide of the aspects may be heterologous to the host cell or to each other. As used herein, “heterologous” in reference to a sequence is a sequence that originates from a foreign species or, if from the same species, is substantially modified from its native form in composition and/or genomic locus by deliberate human intervention. For example, a promoter operably linked to a heterologous polynucleotide is from a species different from the species from which the polynucleotide was derived or, if from the same/analogous species, one or both are substantially modified from their original form and/or genomic locus or the promoter is not the native promoter for the operably linked polynucleotide.

The termination region may be native with the transcriptional initiation region, may be native with the operably linked DNA sequence of interest, may be native with the plant host, or may be derived from another source (i.e., foreign or heterologous to the promoter, the DNA sequence being expressed, the plant host, or any combination thereof). Convenient termination regions are available from the Ti-plasmid of A. tumefaciens, such as the octopine synthase and nopaline synthase termination regions. See also, Guerineau, et al., (1991)Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903; and Joshi, et al., (1987) Nucleic Acid Res. 15:9627-9639, herein incorporated by reference in their entirety.

The expression cassette comprising the sequences of the present disclosure may also contain at least one additional nucleotide sequence for a gene, heterologous nucleotide sequence, heterologous polynucleotide of interest, or heterologous polynucleotide to be cotransformed into the organism. Alternatively, the additional nucleotide sequence(s) can be provided on another expression cassette.

Where appropriate, the nucleotide sequences whose expression is to be under the control of the tissue-preferred promoter sequence of the present disclosure and any additional nucleotide sequence(s) may be optimized for increased expression in the transformed plant. That is, these nucleotide sequences can be synthesized using plant preferred codons for improved expression. See, for example, Campbell and Gowri, (1990) Plant Physiol. 92:1-11, herein incorporated by reference in its entirety, for a discussion of host-preferred codon usage. Methods are available in the art for synthesizing plant-preferred genes. See, for example, U.S. Pat. Nos. 5,380,831, 5,436,391 and Murray, et al., (1989) Nucleic Acids Res. 17:477-498, herein incorporated by reference in their entirety.

Additional sequence modifications are known to enhance gene expression in a cellular host. These include elimination of sequences encoding spurious polyadenylation signals, exon-intron splice site signals, transposon-like repeats and other such well-characterized sequences that may be deleterious to gene expression. The G-C content of the heterologous nucleotide sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include, without limitation: picornavirus leaders, for example, EMCV leader (Encephalomyocarditis 5′ noncoding region) (Elroy-Stein, et al., (1989) Proc. Nat. Acad. Sci. USA 86:6126-6130); potyvirus leaders, for example, TEV leader (Tobacco Etch Virus) (Allison, et al., (1986) Virology 154:9-20); MDMV leader (Maize Dwarf Mosaic Virus); human immunoglobulin heavy-chain binding protein (BiP) (Macejak, et al., (1991) Nature 353:90-94); untranslated leader from the coat protein mRNA of alfalfa mosaic virus (AMV RNA 4) (Jobling, et al., (1987) Nature 325:622-625); tobacco mosaic virus leader (TMV) (Gallie, et al., (1989) Molecular Biology of RNA, pages 237-256) and maize chlorotic mottle virus leader (MCMV) (Lommel, et al., (1991) Virology 81:382-385), herein incorporated by reference in their entirety. See, also, Della-Cioppa, et al., (1987) Plant Physiology 84:965-968, herein incorporated by reference in its entirety. Methods known to enhance mRNA stability can also be utilized, for example, introns, such as the maize Ubiquitin intron (Christensen and Quail, (1996) Transgenic Res. 5:213-218; Christensen, et al., (1992) Plant Molecular Biology 18:675-689) or the maize AdhI intron (Kyozuka, et al., (1991) Mol. Gen. Genet. 228:40-48; Kyozuka, et al., (1990) Maydica 35:353-357) and the like, herein incorporated by reference in their entirety.

The DNA constructs of the aspects can also include further enhancers, either translation or transcription enhancers, as may be required. These enhancer regions are well known to persons skilled in the art, and can include the ATG initiation codon and adjacent sequences. The initiation codon must be in phase with the reading frame of the coding sequence to ensure translation of the entire sequence. The translation control signals and initiation codons can be from a variety of origins, both natural and synthetic. Translational initiation regions may be provided from the source of the transcriptional initiation region, or from the structural gene. The sequence can also be derived from the regulatory element selected to express the gene, and can be specifically modified to increase translation of the mRNA. It is recognized that to increase transcription levels enhancers may be utilized in combination with the promoter regions of the aspects. Enhancers are known in the art and include the SV40 enhancer region, the 35S enhancer element, and the like.

In preparing the expression cassette, the various DNA fragments may be manipulated, to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites or the like. For this purpose, in vitro mutagenesis, primer repair, restriction, annealing, resubstitutions, for example, transitions and transversions, may be involved.

Reporter genes or selectable marker genes may also be included in the expression cassettes of the present disclosure. Examples of suitable reporter genes known in the art can be found in, for example, Jefferson, et al., (1991) in Plant Molecular Biology Manual, ed. Gelvin, et al., (Kluwer Academic Publishers), pp. 1-33; DeWet, et al., (1987) Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330, herein incorporated by reference in their entirety.

Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella, et al., (1983) EMBO 1 2:987-992); methotrexate (Herrera Estrella, et al., (1983) Nature 303:209-213; Meijer, et al., (1991) Plant Mol. Biol. 16:807-820); hygromycin (Waldron, et al., (1985) Plant Mol. Biol. 5:103-108 and Zhijian, et al., (1995) Plant Science 108:219-227); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mot. Biol. 15:127-36); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J. 6:2513-2518), herein incorporated by reference in their entirety.

Other genes that could serve utility in the recovery of transgenic events would include, but are not limited to, examples such as GUS (beta-glucuronidase; Jefferson, (1987) Plant Mot. Biol. Rep. 5:387), GFP (green fluorescence protein; Chalfie, et al., (1994) Science 263:802), luciferase (Riggs, et al., (1987) Nucleic Acids Res. 15(19):8115 and Luehrsen, et al., (1992) Methods Enzymol. 216:397-414) and the maize genes encoding for anthocyanin production (Ludwig, et al., (1990) Science 247:449), herein incorporated by reference in their entirety.

The expression cassette comprising the promoter sequences of the present disclosure operably linked a morphogenic gene and optionally further operably linked to a heterologous nucleotide sequence, a heterologous polynucleotide of interest, a heterologous polynucleotide nucleotide, or a sequence of interest can be used to transform any plant. In this manner, genetically modified plants, plant cells, plant tissue, seed, root and the like can be obtained.

As used herein, “vector” refers to a DNA molecule such as a plasmid, cosmid or bacterial phage for introducing a nucleotide construct, for example, an expression cassette or construct, into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

The methods of the disclosure involve introducing a polypeptide or polynucleotide into a plant. As used herein, “introducing” means presenting to the plant the polynucleotide or polypeptide in such a manner that the sequence gains access to the interior of a cell of the plant. The methods of the disclosure do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, stable transformation methods, transient transformation methods and virus-mediated methods.

A “stable transformation” is a transformation in which the nucleotide construct introduced into a plant integrates into the genome of the plant and is capable of being inherited by the progeny thereof. “Transient transformation” means that a polynucleotide is introduced into the plant and does not integrate into the genome of the plant or a polypeptide is introduced into a plant.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway, et al., (1986) Biotechniques 4:320-334), electroporation (Riggs, et al., (1986) Proc. Natl. Acad. Sci. USA 83:5602-5606), Agrobacterium-mediated transformation (Townsend, et al., U.S. Pat. No. 5,563,055 and Zhao, et al., U.S. Pat. No. 5,981,840), direct gene transfer (Paszkowski, et al., (1984) EMBO J. 3:2717-2722) and ballistic particle acceleration (see, for example, U.S. Pat. Nos. 4,945,050; 5,879,918; 5,886,244; 5,932,782; Tomes, et al., (1995) in Plant Cell, Tissue, and Organ Culture: Fundamental Methods, ed. Gamborg and Phillips (Springer-Verlag, Berlin); McCabe, et al., (1988) Biotechnology 6:923-926) and Lec1 transformation (WO 00/28058). Also see, Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783 and 5,324,646; Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, New York), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotechnology 14:745-750 (maize via Agrobacterium tumefaciens), all of which are herein incorporated by reference in their entirety. Methods and compositions for rapid plant transformation are also found in U.S. 2017/0121722, herein incorporated in its entirety by reference. Vectors useful in plant transformation are found in U.S. patent application Ser. No. 15/765,521, herein incorporated by reference in its entirety.

In specific aspects, the DNA constructs comprising the promoter sequences of the disclosure can be provided to a plant using a variety of transient transformation methods. Such transient transformation methods include, but are not limited to, viral vector systems and the precipitation of the polynucleotide in a manner that precludes subsequent release of the DNA. Thus, transcription from the particle-bound DNA can occur, but the frequency with which it is released to become integrated into the genome is greatly reduced. Such methods include the use of particles coated with polyethylimine (PEI; Sigma # P3143).

In other aspects, the polynucleotide of the disclosure may be introduced into plants by contacting plants with a virus or viral nucleic acids. Generally, such methods involve incorporating a nucleotide construct of the disclosure within a viral DNA or RNA molecule. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931 and Porta, et al., (1996) Molecular Biotechnology 5:209-221, herein incorporated by reference in their entirety.

Methods are known in the art for the targeted insertion of a polynucleotide at a specific location in the plant genome. In one aspect, the insertion of the polynucleotide at a desired genomic location is achieved using a site-specific recombination system. See, for example, WO99/25821, WO99/25854, WO99/25840, WO99/25855 and WO99/25853, all of which are herein incorporated by reference in their entirety. Briefly, the polynucleotide of the disclosure can be contained in transfer cassette flanked by two non-identical recombination sites. The transfer cassette is introduced into a plant having stably incorporated into its genome a target site which is flanked by two non-identical recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick, et al., (1986) Plant Cell Reports 5:81-84, herein incorporated by reference in its entirety. These plants may then be grown, and either pollinated with the same transformed strain or different strains, and the resulting progeny having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a nucleotide construct of the disclosure, for example, an expression cassette of the disclosure, stably incorporated into its genome.

There are a variety of methods for the regeneration of plants from plant tissue. The particular method of regeneration will depend on the starting plant tissue and the particular plant species to be regenerated. The regeneration, development and cultivation of plants from single plant protoplast transformants or from various transformed explants is well known in the art (Weissbach and Weissbach, (1988) In: Methods for Plant Molecular Biology, (Eds.), Academic Press, Inc., San Diego, Calif., herein incorporated by reference in its entirety). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the aspects containing a desired polynucleotide is cultivated using methods well known to one skilled in the art.

The aspects provide compositions for screening compounds that modulate expression within plants. The vectors, cells and plants can be used for screening candidate molecules for agonists and antagonists of the regulatory sequences disclosed herein. For example, a reporter gene can be operably linked to a regulatory sequence and expressed as a transgene in a plant. Compounds to be tested are added and reporter gene expression is measured to determine the effect on promoter activity.

In an aspect, the disclosed methods and compositions can be used to introduce into somatic embryos with increased efficiency and speed polynucleotides useful to target a specific site for modification in the genome of a plant derived from the somatic embryo. Site specific modifications that can be introduced with the disclosed methods and compositions include those produced using any method for introducing site specific modification, including, but not limited to, through the use of gene repair oligonucleotides (e.g. US Publication 2013/0019349), or through the use of double-stranded break technologies such as TALENs, meganucleases, zinc finger nucleases, CRISPR-Cas, and the like. For example, the disclosed methods and compositions can be used to introduce a CRISPR-Cas system into a plant cell or plant, for the purpose of genome modification of a target sequence in the genome of a plant or plant cell, for selecting plants, for deleting a base or a sequence, for gene editing, and for inserting a polynucleotide of interest into the genome of a plant or plant cell. Thus, the disclosed methods and compositions can be used together with a CRISPR-Cas system to provide for an effective system for modifying or altering target sites and nucleotides of interest within the genome of a plant, plant cell or seed. The Cas endonuclease gene is a plant optimized Cas9 endonuclease, wherein the plant optimized Cas9 endonuclease is capable of binding to and creating a double strand break in a genomic target sequence the plant genome.

The Cas endonuclease is guided by the guide nucleotide to recognize and optionally introduce a double strand break at a specific target site into the genome of a cell. The CRISPR-Cas system provides for an effective system for modifying target sites within the genome of a plant, plant cell or seed. Further provided are methods and compositions employing a guide polynucleotide/Cas endonuclease system to provide an effective system for modifying target sites within the genome of a cell and for editing a nucleotide sequence in the genome of a cell. Once a genomic target site is identified, a variety of methods can be employed to further modify the target sites such that they contain a variety of polynucleotides of interest. The disclosed compositions and methods can be used to introduce a CRISPR-Cas system for editing a nucleotide sequence in the genome of a cell. The nucleotide sequence to be edited (the nucleotide sequence of interest) can be located within or outside a target site that is recognized by a Cas endonuclease.

CRISPR loci (Clustered Regularly Interspaced Short Palindromic Repeats) (also known as SPIDRs-SPacer Interspersed Direct Repeats) constitute a family of recently described DNA loci. CRISPR loci consist of short and highly conserved DNA repeats (typically 24 to 40 bp, repeated from 1 to 140 times—also referred to as CRISPR-repeats) which are partially palindromic. The repeated sequences (usually specific to a species) are interspaced by variable sequences of constant length (typically 20 to 58 by depending on the CRISPR locus (WO2007/025097 published Mar. 1, 2007).

CRISPR loci were first recognized in E. coli (Ishino et al. (1987) J. Bacterial. 169:5429-5433; Nakata et al. (1989) J. Bacterial. 171:3553-3556). Similar interspersed short sequence repeats have been identified in Haloferax mediterranei, Streptococcus pyogenes, Anabaena, and Mycobacterium tuberculosis (Groenen et al. (1993) Mol. Microbiol. 10:1057-1065; Hoe et al. (1999) Emerg. Infect. Dis. 5:254-263; Masepohl et al. (1996) Biochim. Biophys. Acta 1307:26-30; Mojica et al. (1995) Mol. Microbiol. 17:85-93). The CRISPR loci differ from other SSRs by the structure of the repeats, which have been termed short regularly spaced repeats (SRSRs) (Janssen et al. (2002) OMICS J. Integ. Biol. 6:23-33; Mojica et al. (2000) Mol. Microbiol. 36:244-246). The repeats are short elements that occur in clusters, that are always regularly spaced by variable sequences of constant length (Mojica et al. (2000) Mol. Microbiol. 36:244-246).

Cas gene includes a gene that is generally coupled, associated or close to or in the vicinity of flanking CRISPR loci. The terms “Cas gene” and “CRISPR-associated (Cas) gene” are used interchangeably herein. A comprehensive review of the Cas protein family is presented in Haft et al. (2005) Computational Biology, PLoS Comput Biol 1 (6): e60. doi:10.1371/journal.pcbi.0010060.

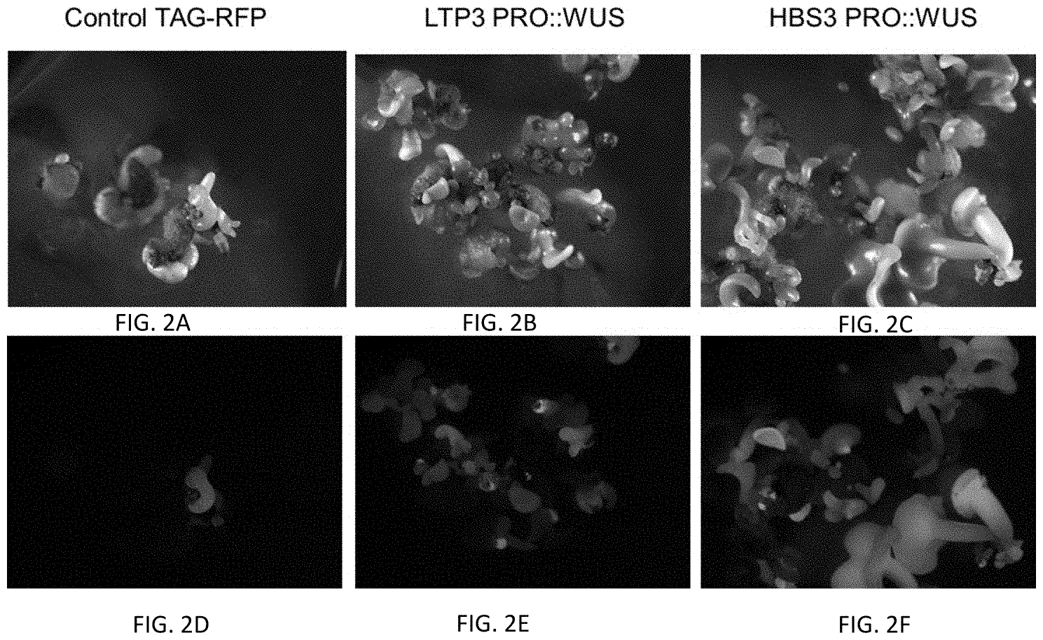

In addition to the four initially described gene families, an additional 41 CRISPR-associated (Cas) gene families have been described in WO/2015/026883, which is incorporated herein by reference. This reference shows that CRISPR systems belong to different classes, with different repeat patterns, sets of genes, and species ranges. The number of Cas genes at a given CRISPR locus can vary between species. Cas endonuclease relates to a Cas protein encoded by a Cas gene, wherein the Cas protein is capable of introducing a double strand break into a DNA target sequence. The Cas endonuclease is guided by the guide polynucleotide to recognize and optionally introduce a double strand break at a specific target site into the genome of a cell. As used herein, the term “guide polynucleotide/Cas endonuclease system” includes a complex of a Cas endonuclease and a guide polynucleotide that is capable of introducing a double strand break into a DNA target sequence. The Cas endonuclease unwinds the DNA duplex in close proximity of the genomic target site and cleaves both DNA strands upon recognition of a target sequence by a guide nucleotide, but only if the correct protospacer-adjacent motif (PAM) is approximately oriented at the 3′ end of the target sequence (see FIG. 2A and FIG. 2B of WO/2015/026883, published Feb. 26, 2015).

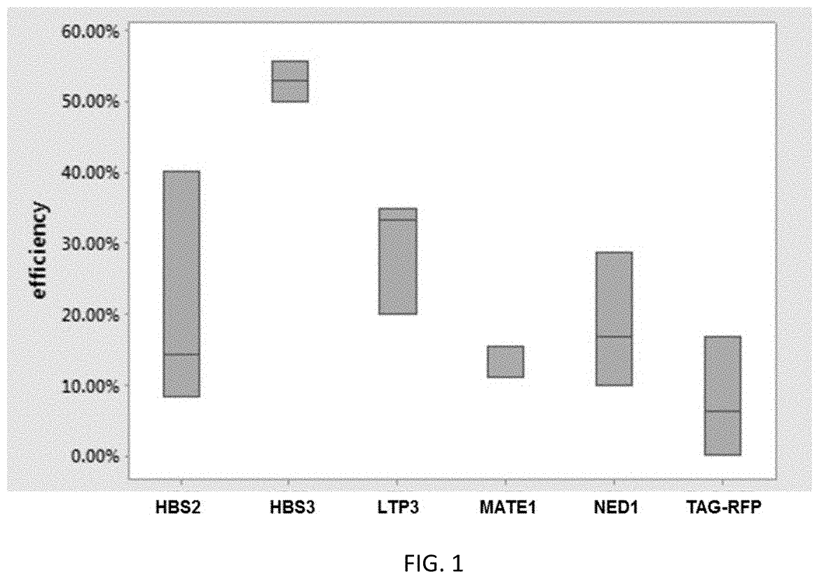

In an aspect, the Cas endonuclease gene is a Cas9 endonuclease, such as, but not limited to, Cas9 genes listed in SEQ ID NOs: 462, 474, 489, 494, 499, 505, and 518 of WO2007/025097, published Mar. 1, 2007, and incorporated herein by reference. In another aspect, the Cas endonuclease gene is plant, maize or soybean optimized Cas9 endonuclease, such as, but not limited to those shown in FIG. 1A of WO/2015/026883. In another aspect, the Cas endonuclease gene is operably linked to a SV40 nuclear targeting signal upstream of the Cas codon region and a bipartite VirD2 nuclear localization signal (Tinland et al. (1992) Proc. Natl. Acad. Sci. USA 89:7442-6) downstream of the Cas codon region.

In an aspect, the Cas endonuclease gene is a Cas9 endonuclease gene of SEQ ID NO:1, 124, 212, 213, 214, 215, 216, 193 or nucleotides 2037-6329 of SEQ ID NO:5, or any functional fragment or variant thereof, of WO/2015/026883.

As related to the Cas endonuclease, the terms “functional fragment,” “fragment that is functionally equivalent,” and “functionally equivalent fragment” are used interchangeably herein. These terms refer to a portion or subsequence of the Cas endonuclease sequence of the present disclosure in which the ability to create a double-strand break is retained.

As related to the Cas endonuclease, the terms “functional variant,” “variant that is functionally equivalent” and “functionally equivalent variant” are used interchangeably herein. These terms refer to a variant of the Cas endonuclease of the present disclosure in which the ability to create a double-strand break is retained. Fragments and variants can be obtained via methods such as site-directed mutagenesis and synthetic construction.

In an aspect, the Cas endonuclease gene is a plant codon optimized Streptococcus pyogenes Cas9 gene that can recognize any genomic sequence of the form N(12-30)NGG can in principle be targeted.

Endonucleases are enzymes that cleave the phosphodiester bond within a polynucleotide chain, and include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex. Endonucleases also include meganucleases, also known as homing endonucleases (HEases), which like restriction endonucleases, bind and cut at a specific recognition site, however the recognition sites for meganucleases are typically longer, about 18 bp or more (Patent application PCT/US 12/30061 filed on Mar. 22, 2012). Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. Meganucleases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI-for enzymes encoded by free-standing ORFs, introns, and inteins, respectively. One step in the recombination process involves polynucleotide cleavage at or near the recognition site. This cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families. TAL effector nucleases are a new class of sequence-specific nucleases that can be used to make double-strand breaks at specific target sequences in the genome of a plant or other organism. (Miller, et al. (2011) Nature Biotechnology 29:143-148). Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a nonspecific endonuclease domain, for example nuclease domain from a Type Ms endonuclease such as Fokl. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3 finger domain recognized a sequence of 9 contiguous nucleotides, with a dimerization requirement of the nuclease, two sets of zinc finger triplets are used to bind an 18 nucleotide recognition sequence.

Bacteria and archaea have evolved adaptive immune defenses termed clustered regularly interspaced short palindromic repeats (CRISPR)/CRISPR-associated (Cas) systems that use short RNA to direct degradation of foreign nucleic acids ((WO2007/025097 published Mar. 1, 2007). The type II CRISPR/Cas system from bacteria employs a crRNA and tracrRNA to guide the Cas endonuclease to its DNA target. The crRNA (CRISPR RNA) contains the region complementary to one strand of the double strand DNA target and base pairs with the tracrRNA (trans-activating CRISPR RNA) forming a RNA duplex that directs the Cas endonuclease to cleave the DNA target.

As used herein, the term “guide nucleotide” relates to a synthetic fusion of two RNA molecules, a crRNA (CRISPR RNA) comprising a variable targeting domain, and a tracrRNA. In an aspect, the guide nucleotide comprises a variable targeting domain of 12 to 30 nucleotide sequences and a RNA fragment that can interact with a Cas endonuclease.

As used herein, the term “guide polynucleotide” relates to a polynucleotide sequence that can form a complex with a Cas endonuclease and enables the Cas endonuclease to recognize and optionally cleave a DNA target site. The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide nucleotide”.

The guide polynucleotide can be a double molecule (also referred to as duplex guide polynucleotide) comprising a first nucleotide sequence domain (referred to as Variable Targeting domain or VT domain) that is complementary to a nucleotide sequence in a target DNA and a second nucleotide sequence domain (referred to as Cas endonuclease recognition domain or CER domain) that interacts with a Cas endonuclease polypeptide. The CER domain of the double molecule guide polynucleotide comprises two separate molecules that are hybridized along a region of complementarity. The two separate molecules can be RNA, DNA, and/or RNA-DNA-combination sequences. In an aspect, the first molecule of the duplex guide polynucleotide comprising a VT domain linked to a CER domain is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the cRNA naturally occurring in Bacteria and Archaea. In an aspect, the size of the fragment of the cRNA naturally occurring in Bacteria and Archaea that is present in a crNucleotide disclosed herein can range from, but is not limited to, 2, 3, 4, 5, 6, 7, 8, 9,10, 11, 12, 13, 14, 15, 16, 17, 18, 19, 20 or more nucleotides.

In an aspect, the second molecule of the duplex guide polynucleotide comprising a CER domain is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides. In an aspect, the RNA that guides the RNA Cas9 endonuclease complex, is a duplexed RNA comprising a duplex crRNA-tracrRNA.

The guide polynucleotide can also be a single molecule comprising a first nucleotide sequence domain (referred to as Variable Targeting domain or VT domain) that is complementary to a nucleotide sequence in a target DNA and a second nucleotide domain (referred to as Cas endonuclease recognition domain or CER domain) that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. In an aspect, the single guide polynucleotide comprises a crNucleotide (comprising a VT domain linked to a CER domain) linked to a tracrNucleotide (comprising a CER domain), wherein the linkage is a nucleotide sequence comprising a RNA sequence, a DNA sequence, or a RNA-DNA combination sequence. The single guide polynucleotide being comprised of sequences from the crNucleotide and tracrNucleotide may be referred to as “single guide nucleotide” (when composed of a contiguous stretch of RNA nucleotides) or “single guide DNA” (when composed of a contiguous stretch of DNA nucleotides) or “single guide nucleotide-DNA” (when composed of a combination of RNA and DNA nucleotides). In an aspect of the disclosure, the single guide nucleotide comprises a cRNA or cRNA fragment and a tracrRNA or tracrRNA fragment of the type II CRISPR/Cas system that can form a complex with a type II Cas endonuclease, wherein the guide nucleotide Cas endonuclease complex can direct the Cas endonuclease to a plant genomic target site, enabling the Cas endonuclease to introduce a double strand break into the genomic target site. One aspect of using a single guide polynucleotide versus a duplex guide polynucleotide is that only one expression cassette needs to be made to express the single guide polynucleotide.

The term “variable targeting domain” or “VT domain” is used interchangeably herein and includes a nucleotide sequence that is complementary to one strand (nucleotide sequence) of a double strand DNA target site. The % complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable target domain can be at least 12, 13, 14, 15, 16, 17, 18, 19, 20, 21, 22, 23, 24, 25, 26, 27, 28, 29 or 30 nucleotides in length. In an aspect, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” of a guide polynucleotide is used interchangeably herein and includes a nucleotide sequence (such as a second nucleotide sequence domain of a guide polynucleotide), that interacts with a Cas endonuclease polypeptide. The CER domain can be composed of a DNA sequence, a RNA sequence, a modified DNA sequence, a modified RNA sequence (see for example modifications described herein), or any combination thereof.

The nucleotide sequence linking the crNucleotide and the tracrNucleotide of a single guide polynucleotide can comprise a RNA sequence, a DNA sequence, or a RNA-DNA combination sequence. In an aspect, the nucleotide sequence linking the crNucleotide and the tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another aspect, the nucleotide sequence linking the crNucleotide and the tracrNucleotide of a single guide polynucleotide can comprise a tetraloop sequence, such as, but not limiting to a GAAA tetraloop sequence.

Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

In an aspect, the guide nucleotide and Cas endonuclease are capable of forming a complex that enables the Cas endonuclease to introduce a double strand break at a DNA target site.

In an aspect of the disclosure the variable target domain is 12, 13, 14, 15, 16, 17, 18, 19, 20, 21, 22, 23, 24, 25, 26, 27, 28, 29 or 30 nucleotides in length.

In an aspect of the disclosure, the guide nucleotide comprises a cRNA (or cRNA fragment) and a tracrRNA (or tracrRNA fragment) of the type II CRISPR/Cas system that can form a complex with a type II Cas endonuclease, wherein the guide nucleotide Cas endonuclease complex can direct the Cas endonuclease to a plant genomic target site, enabling the Cas endonuclease to introduce a double strand break into the genomic target site. The guide nucleotide can be introduced into a plant or plant cell directly using any method known in the art such as, but not limited to, particle bombardment or topical applications.

In an aspect, the guide nucleotide can be introduced indirectly by introducing a recombinant DNA molecule comprising the corresponding guide DNA sequence operably linked to a plant specific promoter that is capable of transcribing the guide nucleotide in the plant cell. The term “corresponding guide DNA” includes a DNA molecule that is identical to the RNA molecule but has a “T” substituted for each “U” of the RNA molecule.

In an aspect, the guide nucleotide is introduced via particle bombardment or using the disclosed methods and compositions for Agrobacterium transformation of a recombinant DNA construct comprising the corresponding guide DNA operably linked to a plant U6 polymerase III promoter.

In an aspect, the RNA that guides the RNA Cas9 endonuclease complex, is a duplexed RNA comprising a duplex crRNA-tracrRNA. One advantage of using a guide nucleotide versus a duplexed crRNA-tracrRNA is that only one expression cassette needs to be made to express the fused guide nucleotide.

The terms “target site,” “target sequence,” “target DNA,” “target locus,” “genomic target site,” “genomic target sequence,” and “genomic target locus” are used interchangeably herein and refer to a polynucleotide sequence in the genome (including choloroplastic and mitochondrial DNA) of a plant cell at which a double-strand break is induced in the plant cell genome by a Cas endonuclease. The target site can be an endogenous site in the plant genome, or alternatively, the target site can be heterologous to the plant and thereby not be naturally occurring in the genome, or the target site can be found in a heterologous genomic location compared to where it occurs in nature.

As used herein, terms “endogenous target sequence” and “native target sequence” are used interchangeably herein to refer to a target sequence that is endogenous or native to the genome of a plant and is at the endogenous or native position of that target sequence in the genome of the plant. In an aspect, the target site can be similar to a DNA recognition site or target site that that is specifically recognized and/or bound by a double-strand break inducing agent such as a LIG3-4 endonuclease (US patent publication 2009-0133152 A1 (published May 21, 2009) or a MS26++meganuclease (U.S. patent application Ser. No. 13/526,912 filed Jun. 19, 2012).

An “artificial target site” or “artificial target sequence” are used interchangeably herein and refer to a target sequence that has been introduced into the genome of a plant. Such an artificial target sequence can be identical in sequence to an endogenous or native target sequence in the genome of a plant but be located in a different position (i.e., a non-endogenous or non-native position) in the genome of a plant.

An “altered target site,” “altered target sequence” “modified target site,” and “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

A summary of SEQ ID NOS: 1-189 is presented in Table 1.

TABLE 1
Summary of SEQ ID NOS: 1-189.

| | Polynucleotide | | |
|---|---|---|---|
| SEQ | (DNA) or | | |
| ID | Polypeptide | | |
| NO. | (PRT) | Name | Description |
| 1 | DNA | GM-HBSTART3 | Glycine max HBSTART3 |
| | | | (Homeodomain StAR-related lipid |
| | | | transfer3) promoter sequence |
| 2 | DNA | GM-HBSTART3 | Glycine max HBSTART3 |
| | | (TRUNCATED) | (Homeodomain StAR-related lipid |
| | | | transfer3) promoter sequence |
| | | | (TRUNCATED) |
| 3 | DNA | AT-ML1 | Arabidopsis thaliana ML1 (MERISTEM |
| | | | LAYER1) promoter sequence |
| 4 | DNA | GM-ML1-Like | Glycine max ML1-Like (MERISTEM |
| | | | LAYER1-Like) promoter sequence |
| 5 | DNA | GM-ML1-Like | Glycine max ML1-Like (MERISTEM |
| | | (TRUNCATED) | LAYER1-Like) promoter sequence |
| | | | (TRUNCATED) |
| 6 | DNA | ZM-HBSTART3 | Zea mays HBSTART3 (Homeodomain |
| | | | StAR-related lipid transfer3) promoter |
| | | | sequence |
| 7 | DNA | OS-HBSTART3 | Oryza sativa HBSTART3 |
| | | | (Homeodomain StAR-related lipid |
| | | | transfer3) promoter sequence |
| 8 | DNA | AT-PDF1 | Arabidopsis thaliana PDF1 |
| | | | (PROTODERMAL FACTOR1) |
| | | | promoter sequence |
| 9 | DNA | GM-PDF1 | Glycine max PDF1 (PROTODERMAL |
| | | | FACTOR1) promoter sequence |
| 10 | DNA | GM-PDF1 | Glycine max PDF1 (PROTODERMAL |
| | | (TRUNCATED) | FACTOR1) promoter sequence |
| | | | (TRUNCATED) |
| 11 | DNA | SB-PDF1 | Sorghum bicolor PDF1 |
| | | | (PROTODERMAL FACTOR1) |
| | | | promoter sequence |
| 12 | DNA | OS-PDF1 | Oryza sativa PDF1 (PROTODERMAL |
| | | | FACTOR1) promoter sequence |
| 13 | DNA | OS-PDF1 | Oryza sativa PDF1 (PROTODERMAL |
| | | (TRUNCATED) | FACTOR1) promoter sequence |
| | | | (TRUNCATED) |
| 14 | DNA | PT-PDF1 | Populus trichocarpa PDF1 |
| | | | (PROTODERMAL FACTOR1) |
| | | | promoter sequence |
| 15 | DNA | PT-PDF1 | Populus trichocarpa PDF1 |
| | | (TRUNCATED) | (PROTODERMAL FACTOR1) |
| | | | promoter sequence (TRUNCATED) |
| 16 | DNA | SI-PDF1 | Setaria italica PDF1 (PROTODERMAL |
| | | | FACTOR1) promoter sequence |
| 17 | DNA | SI-PDF1 | Setaria italica PDF1 (PROTODERMAL |
| | | (TRUNCATED) | FACTOR1) promoter sequence |
| | | | (TRUNCATED) |
| 18 | DNA | AT-PDF2 | Arabidopsis thaliana PDF2 |
| | | | (PROTODERMAL FACTOR2) |
| | | | promoter sequence |
| 19 | DNA | GM-PDF2 | Glycine max PDF2 (PROTODERMAL |
| | | | FACTOR2) promoter sequence |
| 20 | DNA | GM-PDF2 | Glycine max PDF2 (PROTODERMAL |
| | | (TRUNCATED) | FACTOR2) promoter sequence |
| | | | (TRUNCATED) |
| 21 | DNA | ZM-GL1 | Zea mays GL1 (GLABROUS1) promoter |
| | | | sequence |
| 22 | DNA | AT-PDF2a | Arabidopsis thaliana PDF2a |
| | | | (PROTODERMAL FACTOR2a) |
| | | | promoter sequence |
| 23 | DNA | AT-PDF2a | Arabidopsis thaliana PDF2a |
| | | (TRUNCATED) | (PROTODERMAL FACTOR2a) |
| | | | promoter sequence (TRUNCATED) |
| 24 | DNA | GM-PDF2a | Glycine max PDF2a (PROTODERMAL |
| | | | FACTOR2a) promoter sequence |
| 25 | DNA | GM-PDF2a | Glycine max PDF2a (PROTODERMAL |
| | | (TRUNCATED) | FACTOR2a) promoter sequence |
| | | | (TRUNCATED) |
| 26 | DNA | OS-PDF2 | Oryza sativa PDF2 (PROTODERMAL |
| | | | FACTOR2) promoter sequence |
| 27 | DNA | OS-PDF2 | Oryza sativa PDF2 (PROTODERMAL |
| | | (TRUNCATED) | FACTOR2) promoter sequence |
| | | | (TRUNCATED) |
| 28 | DNA | PT-PDF2 | Populus trichocarpa PDF2 |
| | | | (PROTODERMAL FACTOR2) |
| | | | promoter sequence) |
| 29 | DNA | PT-PDF2 | Populus trichocarpa PDF2 |
| | | (TRUNCATED) | (PROTODERMAL FACTOR2) |
| | | | promoter sequence (TRUNCATED) |
| 30 | DNA | VV-PDF2 | Vitis vinifera PDF2 (PROTODERMAL |
| | | | FACTOR2) promoter sequence |
| 31 | DNA | VV-PDF2 | Vitis vinifera PDF2 (PROTODERMAL |
| | | (TRUNCATED) | FACTOR2) promoter sequence |
| | | | (TRUNCATED) |
| 32 | DNA | ZM-PDF2 | Zea mays PDF2 (PROTODERMAL |
| | | | FACTOR2) promoter sequence |
| 33 | DNA | SI-PDF2 | Setaria italica PDF2 (PROTODERMAL |
| | | | FACTOR2) promoter sequence |
| 34 | DNA | SI-PDF2 | Setaria italica PDF2 (PROTODERMAL |
| | | (TRUNCATED) | FACTOR2) promoter |
| | | | sequence(TRUNCATED) |
| 35 | DNA | VV-PDF2a | Vitis vinifera PDF2a (PROTODERMAL |
| | | | FACTOR2a) promoter sequence |
| 36 | DNA | PT-PDF2a | Populus trichocarpa PDF2a |
| | | | (PROTODERMAL FACTOR2a) |
| | | | promoter sequence |
| 37 | DNA | PT-PDF2a | Populus trichocarpa PDF2a |
| | | (TRUNCATED) | (PROTODERMAL FACTOR2a) |
| | | | promoter sequence (TRUNCATED) |
| 38 | DNA | MT-PDF2 | Medicago trunculata PDF2 |
| | | | (PROTODERMAL FACTOR2) |
| | | | promoter sequence |
| 39 | DNA | MT-PDF2 | Medicago trunculata PDF2 |
| | | (TRUNCATED) | (PROTODERMAL FACTOR2) |
| | | | promoter sequence (TRUNCATED) |
| 40 | DNA | AT-HDG2 | Arabidopsis thaliana HDG2 |
| | | | (HOMEODOMAIN GLABROUS2) |
| | | | promoter sequence |
| 41 | DNA | GM-HDG2 | Glycine max HDG2 (HOMEODOMAIN |
| | | | GLABROUS2) promoter sequence |
| 42 | DNA | GM-HDG2 | Glycine max HDG2 (HOMEODOMAIN |
| | | (TRUNCATED) | GLABROUS2) promoter sequence |
| | | | (TRUNCATED) |
| 43 | DNA | SB-HDG2 | Sorghum bicolor HDG2 |
| | | | (HOMEODOMAIN GLABROUS2) |
| | | | promoter sequence |
| 44 | DNA | SB-HDG2 | Sorghum bicolor HDG2 |
| | | (TRUNCATED) | (HOMEODOMAIN GLABROUS2) |
| | | | promoter sequence (TRUNCATED) |
| 45 | DNA | AT-CER6 | Arabidopsis thaliana CER6 |
| | | | (ECERIFERUM6) promoter sequence |
| 46 | DNA | AT-CER60 | Arabidopsis thaliana CER60 |
| | | | (ECERIFERUM60) promoter sequence |
| 47 | DNA | AT-CER60 | Arabidopsis thaliana CER60 |
| | | (TRUNCATED) | (ECERIFERUM60) promoter sequence |
| | | | (TRUNCATED) |
| 48 | DNA | GM-CER6 | Glycine max CER6 (ECERIFERUM6) |
| | | | promoter sequence |
| 49 | DNA | GM-CER6 | Glycine max CER6 (ECERIFERUM6) |
| | | (TRUNCATED) | promoter sequence (TRUNCATED) |
| 50 | DNA | PT-CER6 | Populus trichocarpa CER6 |
| | | | (ECERIFERUM6) promoter sequence |
| 51 | DNA | PT-CER6 | Populus trichocarpa CER6 |
| | | (TRUNCATED) | (ECERIFERUM6) promoter sequence |
| | | | (TRUNCATED) |
| 52 | DNA | VV-CER6 | Vitis vinifera CER6 (ECERIFERUM6) |
| | | | promoter sequence |
| 53 | DNA | VV-CER6 | Vitis vinifera CER6 (ECERIFERUM6) |
| | | (TRUNCATED) | promoter sequence (TRUNCATED) |
| 54 | DNA | SB-CER6 | Sorghum bicolor CER6 |
| | | | (ECERIFERUM6) promoter sequence |
| 55 | DNA | ZM-CER6 | Zea mays CER6 (ECERIFERUM6) |
| | | | promoter sequence |
| 56 | DNA | SI-CER6 | Setaria italica CER6 (ECERIFERUM6) |
| | | | promoter sequence |
| 57 | DNA | SI-CER6 | Setaria italica CER6 (ECERIFERUM6) |
| | | (TRUNCATED) | promoter sequence (TRUNCATED) |
| 58 | DNA | OS-CER6 | Oryza sativa CER6 (ECERIFERUM6) |
| | | | promoter sequence |
| 59 | DNA | OS-CER6 | Oryza sativa CER6 (ECERIFERUM6) |
| | | (TRUNCATED) | promoter sequence (TRUNCATED) |
| 60 | DNA | AT-WUS | Arabidopsis thaliana WUS coding |
| | | | sequence |
| 61 | PRT | AT-WUS | Arabidopsis thaliana WUS protein |
| | | | sequence |
| 62 | DNA | LJ-WUS | Lotus japonicus WUS coding sequence |
| 63 | PRT | LJ-WUS | Lotus japonicus WUS protein sequence |
| 64 | DNA | GM-WUS | Glycine max WUS coding sequence |
| 65 | PRT | GM-WUS | Glycine max WUS protein sequence |
| 66 | DNA | CS-WUS | Camelina sativa WUS coding sequence |
| 67 | PRT | CS-WUS | Camelina sativa WUS protein sequence |
| 68 | DNA | CR-WUS | Capsella rubella WUS coding sequence |
| 69 | PRT | CR-WUS | Capsella rubella WUS protein sequence |
| 70 | DNA | AA-WUS | Arabis alpina WUS coding sequence |
| 71 | PRT | AA-WUS | Arabis alpina WUS protein sequence |
| 72 | DNA | RS-WUS | Raphanus sativus WUS coding sequence |
| 73 | PRT | RS-WUS | Raphanus sativus WUS protein sequence |
| 74 | DNA | BN-WUS | Brassica napus WUS coding sequence |
| 75 | PRT | BN-WUS | Brassica napus WUS protein sequence |
| 76 | DNA | BO-WUS | Brassica oleracea var. oleracea WUS |
| | | | coding sequence |
| 77 | PRT | BO-WUS | Brassica oleracea var. oleracea WUS |
| | | | protein sequence |
| 78 | DNA | HA-WUS | Helianthus annuus WUS coding |
| | | | sequence |
| 79 | PRT | HA-WUS | Helianthus annuus WUS protein |
| | | | sequence |
| 80 | DNA | PT-WUS | Populus trichocarpa WUS coding |
| | | | sequence |
| 81 | PRT | PT-WUS | Populus trichocarpa WUS protein |
| | | | sequence |
| 82 | DNA | VV-WUS | Vitis vinifera WUS coding sequence |
| 83 | PRT | vv-WUS | Vitis vinifera WUS protein sequence |
| 84 | DNA | AT-WUS | Arabidopsis thaliana WUS coding |
| | | | sequence (soy optimized) |
| 85 | PRT | AT-WUS | Arabidopsis thaliana WUS protein |
| | | | sequence |
| 86 | DNA | LJ-WUS | Lotus japonicus WUS coding sequence |
| | | | (soy optimized) |
| 87 | PRT | LJ-WUS | Lotus japonicus WUS protein sequence |
| 88 | DNA | MT-WUS | Medicago trunculata WUS coding |
| | | | sequence (soy optimized) |
| 89 | PRT | MT-WUS | Medicago trunculata WUS protein |
| | | | sequence |
| 90 | DNA | PY-WUS | Petunia hybrida WUS coding sequence |
| | | | (soy optimized) |
| 91 | PRT | PY-WUS | Petunia hybrida WUS protein sequence |
| 92 | DNA | PV-WUS | Phaseolus vulgaris WUS coding |
| | | | sequence (soy optimized) |
| 93 | PRT | PV-WUS | Phaseolus vulgaris WUS protein |
| | | | sequence |
| 94 | DNA | ZM-WUS1 | Zea mays WUS1 coding sequence |
| 95 | PRT | ZM-WUS1 | Zea mays WUS1 protein sequence |
| 96 | DNA | ZM-WUS2 | Zea mays WUS2 coding sequence |
| 97 | PRT | ZM-WUS2 | Zea mays WUS2 protein sequence |
| 98 | DNA | ZM-WUS3 | Zea mays WUS3 coding sequence |
| 99 | PRT | ZM-WUS3 | Zea mays WUS3 protein sequence |
| 100 | DNA | ZM-WOX2A | Zea mays WOX2A coding sequence |
| 101 | PRT | ZM-WOX2A | Zea mays WOX2A protein sequence |
| 102 | DNA | ZM-WOX4 | Zea mays WOX4 coding sequence |
| 103 | PRT | ZM-WOX4 | Zea mays WOX4 protein sequence |
| 104 | DNA | ZM-WOX5A | Zea mays WOX5A coding sequence |
| 105 | PRT | ZM-WOX5A | Zea mays WOX5A protein sequence |
| 106 | DNA | ZM-WOX9 | Zea mays WOX9 coding sequence |
| 107 | PRT | ZM-WOX9 | Zea mays WOX9 protein sequence |
| 108 | DNA | GM-HBSTART2 | Glycine max HBSTART2 |
| | | | (Homeodomain StAR-related lipid |
| | | | transfer2) promoter sequence |
| 109 | DNA | GM-MATE1 | Glycine max MATE1 (Multi- |
| | | | antimicrobial extrusion protein1) |
| | | | promoter sequence |
| 110 | DNA | GM-NED1 | Glycine max NED1 (NAD dependent |
| | | | epimerase/dehydratase1) promoter |
| | | | sequence |
| 111 | DNA | PHP80728 | Synthetic construct comprising the T- |
| | | | DNA (RB to LB) (RB + GM-UBQ |
| | | | PRO::GM-UBQ INTRON1::TAG- |
| | | | RFP::UBQ3 TERM + GM-SAMS |
| | | | PRO::GM-SAMS INTRON1::GM- |
| | | | HRA::GM-ALS TERM + LB) |
| 112 | DNA | PHP80730 | Synthetic construct comprising the T- |
| | | | DNA (RB to LB) (RB + GM-LTP3 |
| | | | PRO::AT-WUS::UBQ14 TERM + GM- |
| | | | UBQ PRO::GM-UBQ INTRON1::TAG- |
| | | | RFP::UBQ3 TERM + GM-SAMS |
| | | | PRO::GM-SAMS INTRON1::GM- |
| | | | HRA::GM-ALS TERM + LB) |
| 113 | DNA | PHP80734 | Synthetic construct comprising the T- |
| | | | DNA (RB to LB) (RB + GM- |
| | | | HBSTART2 PRO::AT-WUS::UBQ14 |
| | | | TERM + GM-UBQ PRO::GM-UBQ |
| | | | INTRON1::TAG-RFP::UBQ3 TERM + |
| | | | GM-SAMS PRO::GM-SAMS |
| | | | INTRON1::GM-HRA::GM-ALS |
| | | | TERM + LB) |
| 114 | DNA | PHP80736 | Synthetic construct comprising the T- |
| | | | DNA (RB to LB) (RB + GM-MATE1 |
| | | | PRO::AT-WUS::UBQ14 TERM + GM- |
| | | | UBQ PRO::GM-UBQ INTRON1::TAG- |
| | | | RFP::UBQ3 TERM + GM-SAMS |
| | | | PRO::GM-SAMS INTRON1::GM- |
| | | | HRA::GM-ALS TERM + LB) |
| 115 | DNA | PHP81060 | Synthetic construct comprising the T- |
| | | | DNA (RB to LB) (RB + GM-NED1 |
| | | | PRO::AT-WUS::UBQ14 TERM + GM- |
| | | | UBQ PRO::GM-UBQ INTRON1::TAG- |
| | | | RFP::UBQ3 TERM + GM-SAMS |
| | | | PRO::GM-SAMS INTRON1::GM- |
| | | | HRA::GM-ALS TERM + LB) |
| 116 | DNA | PHP81343 | Synthetic construct comprising the T- |
| | | | DNA (RB to LB) (RB + GM- |
| | | | HBSTART3 PRO::AT-WUS::UBQ14 |
| | | | TERM + GM-UBQ PRO::GM-UBQ |
| | | | INTRON1::TAG-RFP::UBQ3 TERM + |
| | | | GM-SAMS PRO::GM-SAMS |
| | | | INTRON1::GM-HRA::GM-ALS |
| | | | TERM + LB) |
| 117 | DNA | QC318 | GM-EF1A PRO::GM-EF1A |
| | | | INTRON1::ZS-YELLOW::NOS TERM + |
| | | | GM-SAMS PRO::GM-SAMS |
| | | | INTRON1::GM-ALS::GM-ALS TERM |
| 118 | DNA | pVER9662 | AT-UBIQ10 PRO::AT-UBIQ10 |
| | | | INTRON1::AT-WUS::UBQ14 TERM |
| 119 | DNA | UBIGMWUS | AT-UBIQ10 PRO::AT-UBIQ10 |
| | | | INTRON1::GM-WUS:: UBQ3 TERM |
| 120 | DNA | UBIMTWUS | AT-UBIQ10 PRO::AT-UBIQ10 |
| | | | INTRON1::MT-WUS:: UBQ3 TERM |
| 121 | DNA | UBILJWUS | AT-UBIQ10 PRO::AT-UBIQ10 |
| | | | INTRON1::LJ-WUS:: UBQ3 TERM |
| 122 | DNA | UBIPVWUS | AT-UBIQ10 PRO::AT-UBIQ10 |
| | | | INTRON1::PV-WUS:: UBQ3 TERM |
| 123 | DNA | UBIPYWUS | AT-UBIQ10 PRO::AT-UBIQ10 |
| | | | INTRON1::PY-WUS:: UBQ3 TERM |
| 124 | DNA | GM-LTP3 | Glycine max LTP3 (Lipid Transfer |
| | | | Protein3) promoter sequence |
| 125 | DNA | AT-ML1 | Arabidopsis thaliana ML1 (MERISTEM |
| | | (TRUNCATED) | LAYER1) promoter sequence |
| | | | (TRUNCATED) |
| 126 | DNA | AT-CER6 | Arabidopsis thaliana CER6 |
| | | (TRUNCATED1) | (ECERIFERUM6) promoter sequence |
| | | | (TRUNCATED) |
| 127 | DNA | GG- WUS | Gnetum gnemon WUS coding sequence |
| 128 | PRT | GG- WUS | Gnetum gnemon WUS protein sequence |
| 129 | DNA | MD-WUS | Malus domestica WUS coding sequence |
| 130 | PRT | MD-WUS | Malus domestica WUS protein sequence |
| 131 | DNA | ME-WUS | Manihot esculenta WUS coding |
| | | | sequence |
| 132 | PRT | ME-WUS | Manihot esculenta WUS protein |
| | | | sequence |
| 133 | DNA | KF-WUS | Kalanchoe fedtschenkoi WUS coding |
| | | | sequence |
| 134 | PRT | KF-WUS | Kalanchoe fedtschenkoi WUS protein |
| | | | sequence |
| 135 | DNA | GH-WUS | Gossypium hirsutum WUS coding |
| | | | sequence |
| 136 | PRT | GH-WUS | Gossypium hirsutum WUS protein |
| | | | sequence |
| 137 | DNA | ZOSMA-WUS | Zostera marina WUS coding sequence |
| 138 | PRT | ZOSMA-WUS | Zostera marina WUS protein sequence |
| 139 | DNA | AMBTR-WUS | Amborella trichopoda WUS coding |
| | | | sequence |
| 140 | PRT | AMBTR-WUS | Amborella trichopoda WUS protein |
| | | | sequence |
| 141 | DNA | AC-WUS | Aquilegia coerulea WUS coding |
| | | | sequence |
| 142 | PRT | AC-WUS | Aquilegia coerulea WUS protein |
| | | | sequence |
| 143 | DNA | AH-WUS | Amaranthus hypochondriacus WUS |
| | | | coding sequence |
| 144 | PRT | AH-WUS | Amaranthus hypochondriacus WUS |
| | | | protein sequence |
| 145 | DNA | CUCSA-WUS | Cucumis sativus WUS coding sequence |
| 146 | PRT | CUCSA -WUS | Cucumis sativus WUS protein sequence |
| 147 | DNA | PINTA-WUS | Pinus taeda WUS coding sequence |
| 148 | PRT | PINTA-WUS | Pinus taeda WUS protein sequence |
| 149 | DNA | AT-PDF1 | Arabidopsis thaliana PDF1 |
| | | (TRUNCATED) | (PROTODERMAL FACTOR1) |
| | | | promoter sequence (TRUNCATED) |
| 150 | DNA | AT-PDF2 | Arabidopsis thaliana PDF1 |
| | | (TRUNCATED) | (PROTODERMAL FACTOR1) |
| | | | promoter sequence (TRUNCATED) |
| 151 | DNA | AT-HDG2 | Arabidopsis thaliana HDG2 |
| | | (TRUNCATED) | (HOMEODOMAIN GLABROUS2) |
| | | | promoter sequence (TRUNCATED) |
| 152 | DNA | AT-ANL2 | Arabidopsis thaliana Anthocyanless2 |
| | | | promoter |
| 153 | DNA | RV021090 | Synthetic construct comprising the T- |
| | | | DNA (RB to LB): RB + CAMV35S |
| | | | PRO::HA-WUS::OS-T28 TERM + GM- |
| | | | UBQ PRO::GM-UBQ 5′ UTR::GM- |
| | | | UBQ INTRON1::ZS-YELLOW1 |
| | | | N1::NOS TERM + AT-UBIQ10 |
| | | | PRO::AT-UBIQ10 5′ UTR::AT-UBIQ10 |
| | | | INTRON1::CTP::SPCN::UBQ14 |
| | | | TERM + LB |
| 154 | DNA | RV026522 | T-DNA with GNEGN-WUS (GG - |
| | | | Gnetum gnemon) |
| | | | Synthetic construct comprising the T- |
| | | | DNA (RB to LB): RB + CAMV35S |
| | | | PRO:: GG-WUS::OS-T28 TERM + GM- |
| | | | UBQ PRO::GM-UBQ 5UTR::GM-UBQ |
| | | | INTRON1::ZS-YELLOW1 N1::NOS |
| | | | TERM + AT-UBIQ10 PRO::AT- |
| | | | UBIQ10 5UTR::AT-UBIQ10 |
| | | | INTRON1::CTP::SPCN::UBQ14 |
| | | | TERM + LB |
| 155 | DNA | RV026526 | T-DNA with VITVI-WUS (VV - Vitis |
| | | | vinifera) |
| | | | Synthetic construct comprising the T- |
| | | | DNA (RB to LB): RB + CAMV35S |
| | | | PRO::VV-WUS::OS-T28 TERM + GM- |
| | | | UBQ PRO::GM-UBQ 5′ UTR::GM- |
| | | | UBQ INTRON1::ZS-YELLOW1 |
| | | | N1::NOS TERM + AT-UBIQ10 |
| | | | PRO::AT-UBIQ10 5′ UTR::AT-UBIQ10 |
| | | | INTRON1::CTP::SPCN::UBQ14 |
| | | | TERM + LB |
| 156 | DNA | RV026534 | T-DNA with PETHY-WUS (PH - |
| | | | Petunia hybrida) |
| | | | Synthetic construct comprising the T- |
| | | | DNA (RB to LB): RB + CAMV35S |
| | | | PRO::PH-WUS::OS-T28 TERM + GM- |
| | | | UBQ PRO::GM-UBQ 5′ UTR::GM- |
| | | | UBQ INTRON1::ZS-YELLOW1 |
| | | | N1::NOS TERM + AT-UBIQ10 |
| | | | PRO::AT-UBIQ10 5′ UTR::AT-UBIQ10 |
| | | | INTRON1::CTP::SPCN::UBQ14 |
| | | | TERM + LB |
| 157 | DNA | RV026531 | T-DNA with MALDO-WUS (MD - |
| | | | Malus domestica) |
| | | | Synthetic construct comprising the T- |
| | | | DNA (RB to LB): RB + CAMV35S |
| | | | PRO::MD-WUS::OS-T28 TERM + GM- |
| | | | UBQ PRO::GM-UBQ 5UTR::GM-UBQ |
| | | | INTRON1::ZS-YELLOW1 N1::NOS |
| | | | TERM + AT-UBIQ10 PRO::AT- |
| | | | UBIQ10 5UTR::AT-UBIQ10 |
| | | | INTRON1::CTP::SPCN::UBQ14 |
| | | | TERM + LB |
| 158 | DNA | RV026524 | T-DNA with MANES-WUS (ME - |
| | | | Manihot esculenta) |
| | | | Synthetic construct comprising the T- |
| | | | DNA (RB to LB): RB + CAMV35S |
| | | | PRO::ME-WUS::OS-T28 TERM + GM- |
| | | | UBQ PRO::GM-UBQ 5′ UTR::GM- |
| | | | UBQ INTRON1::ZS-YELLOW1 |
| | | | N1::NOS TERM + AT-UBIQ10 |
| | | | PRO::AT-UBIQ10 5′ UTR::AT-UBIQ10 |
| | | | INTRON1::CTP::SPCN::UBQ14 |
| | | | TERM + LB |
| 159 | DNA | RV026523 | T-DNA with KALFE-WUS (KF - |
| | | | Kalanchoe fedtschenkoi) |
| | | | Synthetic construct comprising the T- |
| | | | DNA (RB to LB): RB + CAMV35S |
| | | | PRO::KF-WUS::OS-T28 TERM + GM- |
| | | | UBQ PRO::GM-UBQ 5′ UTR::GM- |
| | | | UBQ INTRON1::ZS-YELLOW1 |
| | | | N1::NOS TERM + AT-UBIQ10 |
| | | | PRO::AT-UBIQ10 5′ UTR::AT-UBIQ10 |
| | | | INTRON1::CTP::SPCN::UBQ14 |
| | | | TERM + LB |
| 160 | DNA | RV026530 | T-DNA with GOSHI-WUS (GH - |
| | | | Gossypium hirsutum) |
| | | | Synthetic construct comprising the T- |
| | | | DNA (RB to LB): RB + CAMV35S |
| | | | PRO::GH-WUS::OS-T28 TERM + GM- |
| | | | UBQ PRO::GM-UBQ 5′ UTR::GM- |
| | | | UBQ INTRON1::ZS-YELLOW1 |
| | | | N1::NOS TERM + AT-UBIQ10 |
| | | | PRO::AT-UBIQ10 5′ UTR::AT- |
| | | | UBIQ10 |
| | | | INTRON1::CTP::SPCN::UBQ14 |
| | | | TERM + LB |
| 161 | DNA | RV026527 | T-DNA with ZOSMA-WUS (Zostera |
| | | | marina) |
| | | | Synthetic construct comprising the T- |
| | | | DNA (RB to LB): RB + CAMV35S |
| | | | PRO::ZOSMA-WUS::OS-T28 TERM + |
| | | | GM-UBQ PRO::GM-UBQ 5′ |
| | | | UTR::GM-UBQ INTRON1::ZS- |
| | | | YELLOW1 N1::NOS TERM + AT- |
| | | | UBIQ10 PRO::AT-UBIQ10 5′ |
| | | | UTR::AT-UBIQ10 |
| | | | INTRON1::CTP::SPCN::UBQ14 |
| | | | TERM + LB |
| 162 | DNA | RV026529 | T-DNA with AMBTR-WUS (Amborella |
| | | | trichopoda) |
| | | | Synthetic construct comprising the T- |
| | | | DNA (RB to LB): RB + CAMV35S |
| | | | PRO::AMBTR-WUS::OS-T28 TERM + |
| | | | GM-UBQ PRO::GM-UBQ 5′ |
| | | | UTR::GM-UBQ INTRON1::ZS- |
| | | | YELLOW1 N1::NOS TERM + AT- |
| | | | UBIQ10 PRO::AT-UBIQ10 5′ |
| | | | UTR::AT-UBIQ10 |
| | | | INTRON1::CTP::SPCN::UBQ14 |
| | | | TERM + LB |
| 163 | DNA | RV026528 | T-DNA with AQUCO-WUS (Aquilegia |
| | | | coerulea) |
| | | | Synthetic construct comprising the T- |
| | | | DNA (RB to LB): RB + CAMV35S |
| | | | PRO::AC-WUS::OS-T28 TERM + GM- |
| | | | UBQ PRO::GM-UBQ 5′ UTR::GM- |
| | | | UBQ INTRON1::ZS-YELLOW1 |
| | | | N1::NOS TERM + AT-UBIQ10 |
| | | | PRO::AT-UBIQ10 5′ UTR::AT-UBIQ10 |
| | | | INTRON1::CTP::SPCN::UBQ14 |
| | | | TERM + LB |
| 164 | DNA | RV026520 | T-DNA with POPTR-WUS (Populus |
| | | | trichocarpa) |
| | | | Synthetic construct comprising the T- |
| | | | DNA (RB to LB): RB + CAMV35S |
| | | | PRO::PT-WUS::OS-T28 TERM + GM- |
| | | | UBQ PRO::GM-UBQ 5′ UTR::GM- |
| | | | UBQ INTRON1::ZS-YELLOW1 |
| | | | N1::NOS TERM + AT-UBIQ10 |
| | | | PRO::AT-UBIQ10 5; UTR::AT-UBIQ10 |
| | | | INTRON1::CTP::SPCN::UBQ14 |
| | | | TERM + LB |
| 165 | DNA | RV026533 | T-DNA with AMAHY-WUS |
| | | | (Amaranthus hypochondriacus) |
| | | | Synthetic construct comprising the T- |
| | | | DNA (RB to LB): RB + CAMV35S |
| | | | PRO::AH-WUS::OS-T28 TERM + GM- |
| | | | UBQ PRO::GM-UBQ 5′ UTR::GM- |
| | | | UBQ INTRON1::ZS-YELLOW1 |
| | | | N1::NOS TERM + AT-UBIQ10 |
| | | | PRO::AT-UBIQ10 5′ UTR::AT-UBIQ10 |
| | | | INTRON1::CTP::SPCN::UBQ14 |
| | | | TERM + LB |
| 166 | DNA | RV026521 | T-DNA with CUCSA-WUS (Cucumis |
| | | | sativus) |
| | | | Synthetic construct comprising the T- |
| | | | DNA (RB to LB): RB + CAMV35S |
| | | | PRO::CS-WUS::OS-T28 TERM + GM- |
| | | | UBQ PRO::GM-UBQ 5′ UTR::GM- |
| | | | UBQ INTRON1::ZS-YELLOW1 |
| | | | N1::NOS TERM + AT-UBIQ10 |
| | | | PRO::AT-UBIQ10 5′ UTR::AT-UBIQ10 |
| | | | INTRON1::CTP::SPCN::UBQ14 |
| | | | TERM + LB |
| 167 | DNA | RV026525 | T-DNA with PINTA-WUS (Pinus taeda) |
| | | | Synthetic construct comprising the T- |
| | | | DNA (RB to LB): RB + CAMV35S |
| | | | PRO::PINTA-WUS::OS-T28 TERM + |
| | | | GM-UBQ PRO::GM-UBQ 5′ |
| | | | UTR::GM-UBQ INTRON1::ZS- |
| | | | YELLOW1 N1::NOS TERM + AT- |
| | | | UBIQ10 PRO::AT-UBIQ10 5′ |
| | | | UTR::AT-UBIQ10 |
| | | | INTRON1::CTP::SPCN::UBQ14 |
| | | | TERM + LB |
| 168 | DNA | RV022814 | T-DNA with NO WUS cassette |
| | | | Synthetic construct comprising the T- |
| | | | DNA (RB to LB): RB + CCDB + GM- |
| | | | UBQ PRO-V1::GM-UBQ 5′ UTR::GM- |
| | | | UBQ INTRON1::CTP::SPCN::UBQ14 |
| | | | TERM + GM-EF1A2 PRO:: GM-EF1A2 |
| | | | 5′ UTR:: GM-EF1A2 INTRON1:: GM- |
| | | | EF1A2 5′ UTR::DS-RED2::UBQ3 |
| | | | TERM + LB |
| 169 | DNA | RV027344 | T-DNA with AT-PDF2 PRO |
| | | | Synthetic construct comprising the T- |
| | | | DNA (RB to LB): RB + AT-PDF2 |
| | | | PRO::HA-WUS::OS-T28 TERM + GM- |
| | | | UBQ PRO::GM-UBQ 5′ UTR::GM- |
| | | | UBQ INTRON1::ZS-YELLOW1 |
| | | | N1::NOS TERM + AT-UBIQ10 |
| | | | PRO::AT-UBIQ10 5′ UTR::AT-UBIQ10 |
| | | | INTRON1::CTP::SPCN::UBQ14 |
| | | | TERM + LB |
| 170 | DNA | RV026532 | T-DNA for PRO TEST NEG-NO WUS |
| | | | Synthetic construct comprising the T- |
| | | | DNA (RB to LB): RB + GM-UBQ |
| | | | PR0-V1::GM-UBQ 5′ UTR::GM-UBQ |
| | | | INTRON1::ZS-YELLOW1 N1::NOS |
| | | | TERM + GM-UBQ PR0-V1::GM-UBQ |
| | | | 5′ UTR::GM-UBQ |
| | | | INTRON1::CTP::SPCN::UBQ14 TERM + |
| | | | GM-EF1A2 PRO:: GM-EF1A2 5′ |
| | | | UTR:: GM-EF1A2 INTR0N1:: GM- |
| | | | EF1A2 5′ UTR::DS-RED2::UBQ3 |
| | | | TERM + LB |
| 171 | DNA | RV027337 | T-DNA with AT-ANL2 PRO |
| | | | Synthetic construct comprising the T- |
| | | | DNA (RB to LB): RB + AT-ANL2 |
| | | | PRO::HA-WUS::OS-T28 TERM + GM- |
| | | | UBQ PRO::GM-UBQ 5′ UTR::GM- |
| | | | UBQ INTRON1::ZS-YELLOW1 |
| | | | N1::NOS TERM + AT-UBIQ10 |
| | | | PRO::AT-UBIQ10 5′ UTR::AT-UBIQ10 |
| | | | INTRON1::CTP::SPCN::UBQ14 |
| | | | TERM + LB |
| 172 | DNA | RV027338 | T-DNA with AT-CER6 PRO |
| | | | Synthetic construct comprising the T- |
| | | | DNA (RB to LB): RB + AT-CER6 |
| | | | PRO::HA-WUS::OS-T28 TERM + GM- |
| | | | UBQ PRO::GM-UBQ 5′ UTR::GM- |
| | | | UBQ INTRON1::ZS-YELLOW1 |
| | | | N1::NOS TERM + AT-UBIQ10 |
| | | | PRO::AT-UBIQ10 5′ UTR::AT-UBIQ10 |
| | | | INTRON1::CTP::SPCN::UBQ14 |
| | | | TERM + LB |
| 173 | DNA | RV027340 | T-DNA with AT-HDG2 PRO |
| | | | Synthetic construct comprising the T- |
| | | | DNA (RB to LB): RB + AT-HDG2 |
| | | | PRO::HA-WUS::OS-T28 TERM + GM- |
| | | | UBQ PRO::GM-UBQ 5′ UTR::GM- |
| | | | UBQ INTRON1::ZS-YELLOW1 |
| | | | N1::NOS TERM + AT-UBIQ10 |
| | | | PRO::AT-UBIQ10 5′ UTR::AT-UBIQ10 |
| | | | INTRON1::CTP::SPCN::UBQ14 |
| | | | TERM + LB |
| 174 | DNA | RV027342 | T-DNA with AT-ML1 PRO |
| | | | Synthetic construct comprising the T- |
| | | | DNA (RB to LB): RB + AT-ML1 |
| | | | PRO::HA-WUS::OS-T28 TERM + GM- |
| | | | UBQ PRO::GM-UBQ 5′ UTR::GM- |
| | | | UBQ INTRON1::ZS-YELLOW1 |
| | | | N1::NOS TERM + AT-UBIQ10 |
| | | | PRO::AT-UBIQ10 5′ UTR::AT-UBIQ10 |
| | | | INTRON1::CTP::SPCN::UBQ14 |
| | | | TERM + LB |
| 175 | DNA | RV027343 | T-DNA with AT-PDF1 PRO |
| | | | Synthetic construct comprising the T- |
| | | | DNA (RB to LB): RB + AT-PDF1 |
| | | | PRO::HA-WUS::OS-T28 TERM + GM- |
| | | | UBQ PRO::GM-UBQ 5′ UTR::GM- |
| | | | UBQ INTRON1::ZS-YELLOW1 |
| | | | N1::NOS TERM + AT-UBIQ10 |
| | | | PRO::AT-UBIQ10 5′ UTR::AT-UBIQ10 |
| | | | INTRON1::CTP::SPCN::UBQ14 |
| | | | TERM + LB |
| 176 | DNA | PCR Target1 | Synthetic sequence PCR Target1 |
| 177 | DNA | PCR Target2 | Synthetic sequence PCR Target2 |
| 178 | DNA | PCR Target3 | Synthetic sequence PCR Target3 |
| 179 | DNA | PCR Target4 | Synthetic sequence PCR Target4 |
| 180 | DNA | PCR Target5 | Synthetic sequence PCR Target5 |
| 181 | DNA | PCR Target6 | Synthetic sequence PCR Target6 |
| 182 | DNA | PCR Target7 | Synthetic sequence PCR Target7 |
| 183 | DNA | PCR Target8 | Synthetic sequence PCR Target8 |
| 184 | DNA | PCR Target9 | Synthetic sequence PCR Target9 |
| 185 | DNA | PCR Target10 | Synthetic sequence PCR Target10 |
| 186 | DNA | PCR Target11 | Synthetic sequence PCR Target11 |
| 187 | DNA | PCR Target12 | Synthetic sequence PCR Target12 |
| 188 | DNA | PCR Target13 | Synthetic sequence PCR Target13 |
| 189 | DNA | AT-CER6 | Arabidopsis thaliana CER6 |
| | | (TRUNCATED2) | (ECERIFERUM6) promoter sequence |
| | | | (TRUNCATED2) |

The following examples are offered by way of illustration and not by way of limitation.

### EXAMPLES

The aspects of the disclosure are further defined in the following Examples, in which parts and percentages are by weight and degrees are Celsius, unless otherwise stated. These Examples, while indicating aspects of the disclosure, are given by way of illustration only. From the above discussion and these Examples, one skilled in the art can ascertain the essential characteristics of the aspects of the disclosure, and without departing from the spirit and scope thereof, can make various changes and modifications of them to adapt to various usages and conditions. Thus, various modifications in addition to those shown and described herein will be apparent to those skilled in the art from the foregoing description. Such modifications are also intended to fall within the scope of the appended claims.

### Example 1. Plant Materials and Media Compositions

A wide range of tissue or explant types can be used in the current method, including suspension cultures, immature cotyledons, mature cotyledons, split seed, embryonic axes, hypocotyls, epicotyls and leaves. The compositions of various media used in soybean transformation, tissue culture and regeneration are outlined in Table 2. In this table, medium M1 is used for initiation of suspension cultures, if this is the starting material for transformation. Media M2 and M3 represent typical co-cultivation media useful for Agrobacterium transformation of the entire range of explants listed above. Medium M4 is useful for selection (with the appropriate selective agent), M5 is used for somatic embryo maturation, and medium M6 is used for germination to produce T0 plantlets.

TABLE 2
Composition of Cultivation Media M1-M5

| | M1 | | M2 | | M3 | | M4 | | M5 | M6 |
|---|---|---|---|---|---|---|---|---|---|---|
| MS salt with | 4.44 | g/L | | | | | 4.4 | g/L | 4.44 g/L | |
| B5 vitamins | | | | | | | | | | |
| (PhytoTech | | | | | | | | | | |
| M404) | | | | | | | | | | |
| Gamborg B-5 | | | | | | | | | | 3.21 g/L |
| basal medium | | | | | | | | | | |
| (PhytoTech | | | | | | | | | | |
| G398) | | | | | | | | | | |
| Modified MS | | | 2.68 | g/L | 2.68 | g/L | | | | |
| salt | | | | | | | | | | |
| (PhytoTech | | | | | | | | | | |
| M571) | | | | | | | | | | |
| B5 vitamins | | | 1 | ml | 1 | ml | 1 | ml | | |
| (1000X) | | | | | | | | | | |
| (PhytoTech | | | | | | | | | | |
| G249) | | | | | | | | | | |
| 2,4-D stock | 4 | ml | 1 | ml | 1 | ml | 4 | ml | | |
| 10 mg/ml | | | | | | | | | | |
| KNO₃ | | | 1.64 | g/L | 1.64 | g/L | | | | |
| (NH₄)₂SO₄ | | | 0.463 | g/L | 0.463 | g/L | | | | |
| Asparagine | | | 1 | g/L | 1 | g/L | | | | |
| Sucrose | | | 68.5 | g/L | 85.6 | g/L | | | | 20 g/L |
| Glucose | 31.5 | g/L | 36 | g/L | 49.6 | g/L | 31.5 | g/L | | |
| Maltose | | | | | | | | | 60 g/L | |
| MgCl₂•6H₂O | | | | | | | | | 0.75 g/L | |
| Activated | | | | | | | | | 5 g/L | |
| charcoal | | | | | | | | | | |
| (PhytoTech | | | | | | | | | | |
| C325) | | | | | | | | | | |
| Casein | | | 1 | g/L | 1 g/L | g/L | | | | |
| hydrolysate | | | | | | | | | | |
| (PhytoTech | | | | | | | | | | |
| C184) | | | | | | | | | | |
| pH | 7.0 | | 5.4 | | 5.4 | | 7.0 | | 5.7 | 5.7 |
| Acetosyringone | | | 300 | μM | 300 | μM | | | | |
| TC agar | 4 | g/L | | | | | | | | 5 g/L |
| Gelrite (Plant | | | | | | | 2 | g/L | 2 g/L | |
| Media Cat# | | | | | | | | | | |
| 714246) | | | | | | | | | | |

After 1-5 days of co-culture, the tissue is cultured on M3 medium with no selection for one week (recovery period), and then moved onto selection. For selection, an antibiotic or herbicide is added to M3 medium for the selection of stable transformants. To begin counter-selection against Agrobacterium, 300 mg/l Timentin® (sterile ticarcillin disodium mixed with clavulanate potassium, PlantMedia, Dublin, Ohio, USA) is also added, and both the selective agent and Timentin® are maintained in the medium throughout selection (up to total 8 weeks). The selective media is replaced weekly. After 6-8 weeks on selective medium, transformed tissue becomes visible as green tissue against a background of bleached (or necrotic), less healthy tissue. These pieces of tissue are cultured for an additional 4-8 weeks.

Green and healthy somatic embryos are then transferred to M5 media containing 100 mg/L Timentin®. After a total of 4 weeks of maturation on M5 media, mature somatic embryos are placed in a sterile, empty Petri dish, sealed with Micropore™ tape (3M Health Care, St. Paul, Minn., USA) or placed in a plastic box (with no fiber tape) for 4-7 days at room temperature.

Desiccated embryos are planted in M6 media where they are left to germinate at 26° C. with an 18-hour photoperiod at 60-100 μE/m²/s light intensity. After 4-6 weeks in germination media, the plantlets are transferred to moistened Jiffy-7 peat pellets (Jiffy Products Ltd, Shippagan, Canada), and kept enclosed in clear plastic tray boxes until acclimatized in a Percival incubator under the following conditions, a 16-hour photoperiod at 60-100 μE/m²/s, 26° C./24° C. day/night temperatures. Finally, hardened plantlets are potted in 2 gallon pots containing moistened SunGro 702 and grown to maturity, bearing seed, in a greenhouse.

### Example 2. Transformation of Soybean

Standard protocols for particle bombardment (Finer and McMullen, 1991, In Vitro Cell Dev. Biol.—Plant 27:175-182), Agrobacterium-mediated transformation (Jia et al., 2015, Int J. Mol. Sci. 16:18552-18543; US20170121722 incorporated herein by reference in its entirety), or Ochrobactrum-mediated transformation (US20180216123 incorporated herein by regerence in its entirety) for soybean can be used with the methods of the disclosure.

### Example 3. The GM-LTP3 Promoter Controlling WUS Expression Improves Soy Transformation

The Agrobacterium strain AGL1, containing a T-DNA with the expression cassette GM-LTP3 PRO::AT-WUS::UBQ14 TERM+GM-UBQ PRO::GM-UBQ INTRON1::TAG-RFP::UBQ3 TERM+GM-SAMS PRO::GM-SAMS INTRON1::GM-HRA::GM-ALS TERM (PHP80730; SEQ ID NO:112), was used to transform Pioneer soybean variety PHY21. Four days after the Agrobacterium infection was started, the tissue was washed with sterile culture medium to remove excess bacteria. Nine days later the tissue was moved to somatic embryo maturation medium, and twenty-two days after that the transgenic somatic embryos were ready for dry-down. At this point, well-formed, mature somatic embryos were fluorescing red under an epifluorescence stereo-microscope with an RFP filter set. The somatic embryos that developed were functional and germinated to produce healthy plants in the greenhouse. This rapid method of producing somatic embryos and germinating to form plants reduced the typical timeframe from Agrobacterium infection to moving transgenic T0 plants into the greenhouse from four months (for conventional soybean transformation) to two months.

### Example 4. The GM-HBSTART3 Promoter Controlling WUS Expression Improves Soy Transformation Maturation Efficiency

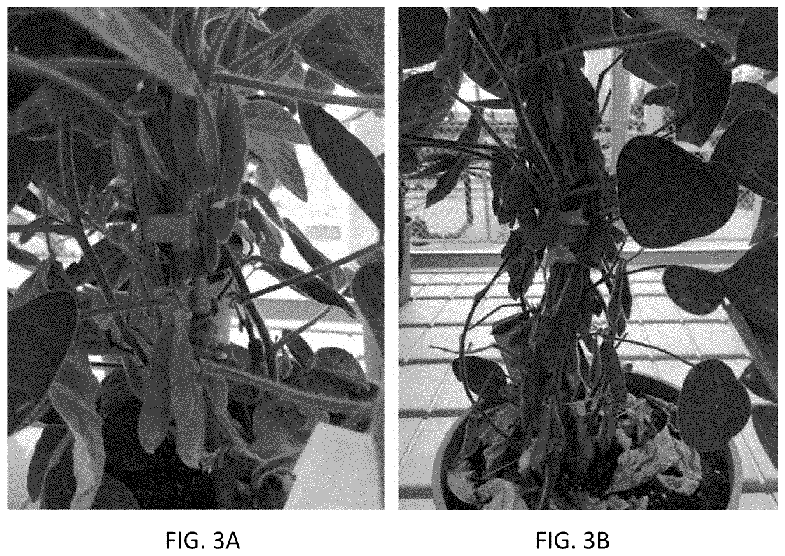

In soybean varieties that are difficult to transform, it is commonly observed that many of the transgenic somatic embryos that do form during the soybean transformation process fail to mature properly. Failure of the somatic embryos to mature ultimately results in greatly reduced frequencies of germination to form transgenic plantlets that will survive in the greenhouse. Promoters (GM-HBSTART3 (SEQ ID NO:1), GM-LTP3 (SEQ ID NO:124), GM-HBSTART2 (SEQ ID NO:108), GM-MATE1(SEQ ID NO:109), GM-NED1 (SEQ ID NO:110)) driving WUS expression were tested to determine their impact on soy transformation maturation efficiency. The GM-HBSTART3 promoter (SEQ ID NO:1) driving WUS expression (PHP81343; SEQ ID NO:116) greatly improved the efficiency of somatic embryo maturation, increasing maturation efficiency from a median value of less than 8% in the TAG-RFP control (PHP80728; SEQ ID NO:111) to over 50%. See FIG. 1. This directly translated into an increased frequency of transgenic mature somatic embryos that were capable of germinating to form transgenic T0 plants. See FIGS. 3A and 3B.

Using other promoters to drive expression of WUS resulted in intermediate maturation efficiencies, with median levels of 13%, 33%, 13% and 15% for the GM-HBSTART2 (PHP80734; SEQ ID NO:113), GM-LTP3 (PHP80730; SEQ ID NO:112), GM-MATE1 (PHP80736; SEQ ID NO:114) or the GM-NED1(PHP81060; SEQ ID NO:115) promoters, respectively. See FIG. 1. In the TAG-RFP control treatment (PHP80728; SEQ ID NO:111), somatic embryos were small and under-developed at 5-6 weeks after Agrobacterium-infection, while the somatic embryos in the GM-LTP3 PRO::WUS (PHP80730; SEQ ID NO:112) and GM-HBSTART3 PRO::WUS (PHP81343; SEQ ID NO:116) treatments were approximately 2 to 5-fold larger, respectively. See FIG. 2A-2F.

### Example 5. The GM-HBSTART3 or the GM-LTP3 Promoter Controlling Expression of Morphogenic Genes Improves Transformation

Using the GM-HBSTART3 promoter (SEQ ID NO: 1), the GM-LTP3 promoter (SEQ ID NO:124) or any of the other promoters disclosed herein to drive expression of an Arabidopsis LEC1, LEC2, KN1, STM or LEC1-like (Kwong et al., (2003) The Plant Cell, Vol. 15, 5-18) gene in expression cassettes comprising a fluorescent marker, is found to increase the frequency of somatic embryo formation and the recovery of transgenic T0 plants. Agrobacterium strain LBA4404 is used to transform various explant types, including immature cotyledons, split seeds, isolated embryonic axes, mature cotyledonary nodes, hypocotyl, epicotyl or leaf tissue of Pioneer soybean variety PHY21. Four days after the Agrobacterium infection is started, the tissue is washed with sterile culture medium to remove excess bacteria. It is expected that approximately nine days later the tissue is moved to somatic embryo maturation medium, and approximately twenty-two days after that the transgenic somatic embryos are ready for dry-down. At this point, well-formed, mature somatic embryos fluoresce under an epifluorescence stereo-microscope with an appropriate filter set. The somatic embryos that develop are functional and germinate to produce healthy plants in the greenhouse. It is expected that this rapid method of producing somatic embryos and germinating to form plants reduces the typical timeframe from Agrobacterium infection to moving transgenic T0 plants into the greenhouse from four months (for conventional soybean transformation) to approximately two to three months.

### Example 6. Use of the GM-HBSTART3 or the GM-LTP3 Promoter to Control Expression of the Agrobacterium IPT Gene Stimulates Direct Shoot Formation and Improves Transformation

Using the GM-HBSTART3 promoter (SEQ ID NO: 1), the GM-LTP3 promoter (SEQ ID NO:124) or any of the other promoters disclosed herein to drive expression of an Agrobacterium IPT gene in expression cassettes comprising a fluorescent marker, is found to increase the frequency of multiple shoot formation and the recovery of transgenic T0 plants. Agrobacterium strain LBA4404 is used to transform various explant types, including immature cotyledons, split seeds, isolated embryonic axes, mature cotyledonary nodes, hypocotyl, epicotyl or leaf tissue of Pioneer soybean variety PHY21. Four days after the Agrobacterium infection is started, the tissue is washed with sterile culture medium to remove excess bacteria and moved onto medium that promotes multiple shoot proliferation. It is expected that nine days later the tissue is moved to medium that favors shoot development, and twenty-two after that the transgenic shoots are moved onto medium that promotes rooting. At this point, incipient plantlets fluoresce under an epifluorescence stereo-microscope with an appropriate filter set. Functional plantlets develop rapidly and continue to grow and produce healthy plants in the greenhouse. It is expected that this rapid method of directly forming transgenic plants reduces the typical timeframe from Agrobacterium infection to moving transgenic T0 plants into the greenhouse from four months (for conventional soybean transformation) to approximately two to three months.

### Example 7. The GM-HBSTART3 or the GM-LTP3 Promoter Controlling Expression of the Arabidopsis Monopteros-Delta Gene Stimulates Direct Shoot Formation and Improves Transformation

Using the GM-HBSTART3 promoter (SEQ ID NO: 1), the GM-LTP3 promoter (SEQ ID NO:124) or any of the other promoters disclosed herein to drive expression of an Arabidopsis MONOPTEROS-DELTA gene in expression cassettes comprising a fluorescent marker, is found to increase the frequency of multiple shoot formation and the recovery of transgenic T0 plants. Agrobacterium strain LBA4404 is used to transform various explant types, including immature cotyledons, split seeds, isolated embryonic axes, mature cotyledonary nodes, hypocotyl, epicotyl or leaf tissue in the Pioneer soybean variety PHY21. Four days after the Agrobacterium infection is started, the tissue is washed with sterile culture medium to remove excess bacteria and moved onto medium that promotes multiple shoot proliferation. It is expected that nine days later the tissue is moved to medium that favors shoot development, and twenty-two days after that the transgenic shoots are moved onto medium that promotes rooting. At this point, incipient plantlets fluoresce under an epifluorescence stereo-microscope with an appropriate filter set. Functional plantlets develop rapidly and continue to grow and produce healthy plants in the greenhouse. It is expected that this rapid method of directly forming transgenic plants reduces the typical timeframe from Agrobacterium infection to moving transgenic T0 plants into the greenhouse from four months (for conventional soybean transformation) to approximately two to three months.

### Example 8. The GM-HBSTART3 or the GM-LTP3 Promoter Controlling Expression of Growth Enhancing Genes Improves Transformation

Using the GM-HBSTART3 promoter (SEQ ID NO: 1), the GM-LTP3 promoter (SEQ ID NO:124) or any of the other promoters disclosed herein to drive expression of an Agrobacterium AV-6B gene, an Agrobacterium IAA-h gene, an Agrobacterium IAA-m gene, an Arabidopsis SERK or an Arabidopsis AGL15 gene in expression cassettes comprising a fluorescent marker, is found to increase the frequency of somatic embryo formation and the recovery of transgenic T0 plants. Agrobacterium strain LBA4404 is used to transform various explant types, including immature cotyledons, split seeds, isolated embryonic axes, mature cotyledonary nodes, hypocotyl, epicotyl or leaf tissue of Pioneer soybean variety PHY21. Four days after the Agrobacterium infection is started, the tissue is washed with sterile culture medium to remove excess bacteria. It is expected that nine days later the tissue is moved to somatic embryo maturation medium, and twenty-two days after that the transgenic somatic embryos are ready for dry-down. At this point, well-formed, mature somatic embryos fluoresce under an epifluorescence stereo-microscope with an appropriate filter set. The somatic embryos that develop are functional and germinate to produce healthy plants in the greenhouse. It is expected that this rapid method of producing somatic embryos and germinating to form plants reduces the typical timeframe from Agrobacterium infection to moving transgenic T0 plants into the greenhouse from four months (for conventional soybean transformation) to approximately two to three months.

### Example 9. Use of Viral Enhancer Elements in Conjunction with the GM-HBSTART3 Promoter T0 Drive Expression of WUS Results in Further Improvement of Soy Transformation

Relative to using the GM-HBSTART3 promoter (SEQ ID NO: 1), the GM-LTP3 promoter (SEQ ID NO:124) or any of the other promoters disclosed herein alone, use of a viral enhancer element such as the 35S enhancer adjacent to the GM-HBSTART3 promoter (SEQ ID NO: 1), the GM-LTP3 promoter (SEQ ID NO:124) or any of the other promoters disclosed herein to drive expression of WUS in expression cassettes comprising a fluorescent marker, results in a further increase in the frequency of somatic embryo formation and the frequency of somatic embryo maturation, resulting in an overall increase in the recovery of transgenic T0 plants. Agrobacterium strain LBA4404 is used to transform various explant types, including immature cotyledons, split seeds, isolated embryonic axes, mature cotyledonary nodes, hypocotyl, epicotyl or leaf tissue of Pioneer soybean variety PHY21. Four days after the Agrobacterium infection is started, the tissue is washed with sterile culture medium to remove excess bacteria. Nine days later the tissue is moved to somatic embryo maturation medium, and twenty-two days after that transgenic somatic embryos are ready for dry-down. At this point, well-formed, mature somatic embryos fluoresce under an epifluorescence stereo-microscope with an appropriate filter set. The somatic embryos that develop are functional and germinate to produce healthy plants in the greenhouse. This rapid method of producing somatic embryos and germinating to form plants reduces the typical timeframe from Agrobacterium infection to moving transgenic T0 plants into the greenhouse from four months (for conventional soybean transformation) to approximately two to three months.

Other enhancer elements are tested in a similar fashion, and are shown to also result in increased transformation relative to using the GM-HBSTART3 promoter (SEQ ID NO: 1) alone. These enhancers include the viral enhancers such as the Cauliflower Mosaic Virus 35S and the Mirabilis Mosaic Virus 2×MMV as well as the endogenous plant enhancer elements.

### Example 10. Orthologs of the Arabidopsis WUS Gene Function in Soybean T0 Stimulate Somatic Embryo Formation

The following treatments were compared. For the particle gun transformation treatments, all contained plasmid QC318 (SEQ ID NO: 117) with GM-EF1A PRO::GM-EF1A INTRON1::ZS-YELLOW::NOS TERM+GM-SAMS PRO::GM-SAMS INTRON1::GM-ALS::GM-ALS TERM. The treatments included 1) a control with no addition genes, 2) pVER9662 (SEQ ID NO: 118) with the AT-UBI PRO driving expression of the Arabidopsis WUS gene, 3) UBIGMWUS (SEQ ID NO: 119) with the AT-UBI PRO driving expression of the Glycine max WUS gene, 4) UBIMTWUS (SEQ ID NO: 120) with the AT-UBI PRO driving expression of the Medicago truncatula WUS gene, 5) UBILJWUS (SEQ ID NO: 121) with the AT-UBI PRO driving expression of the Lotus japonica WUS gene, 6) UBIPVWUS (SEQ ID NO: 122) with the AT-UBI PRO driving expression of the Phaseolus vulgaris WUS gene, and 7) UBIPYWUS (SEQ ID NO: 123) with the AT-UBI PRO driving expression of the petunia WUS gene.

Immature cotyledons were isolated from the seed, pre-cultured for two weeks, and then transformed with the particle gun, co-introducing a mixture of two plasmids, the first containing an expression cassette consisting of the AT-UBI PRO driving expression of the cDNA sequence for each of the WUS orthologs (pVER9662 (SEQ ID NO: 118), UBIGMWUS (SEQ ID NO: 119), UBIMTWUS (SEQ ID NO: 120), UBILJWUS (SEQ ID NO: 121), UBIPVWUS (SEQ ID NO: 122), and UBIPYWUS (SEQ ID NO: 123)) plus an expression cassette for ZS-YELLOW (QC318 (SEQ ID NO: 117)). The explants were cultured for two weeks. At two weeks, the number of fluorescing globular somatic embryos were counted and tabulated for each treatment.

Two weeks after particle gun transformation, fluorescent globular somatic embryos were rarely observed on the bombarded control cotyledons, while for all the other treatments (containing WUS genes from different dicot species driven by the AT-UBI promoter), numerous fluorescent somatic embryos were observed on bombarded cotyledons.

### Example 11. Orthologs of the Arabidopsis WUS Gene Function in Brassica T0 Stimulate Somatic Embryo Formation

Various explant types, including immature cotyledons, split seeds, isolated embryonic axes, mature cotyledonary nodes, hypocotyl, epicotyl or leaf tissue of Brassica are isolated for transformation with orthologs of the Arabidopsis WUS gene. The following treatments are compared. For the particle gun transformation treatments, all contain plasmid QC318 (SEQ ID NO: 117) with GM-EF1A PRO::GM-EF1A INTRON1::ZS-YELLOW::NOS TERM+GM-SAMS PRO::GM-SAMS INTRON1::GM-ALS::GM-ALS TERM. The treatments include 1) a control with no addition genes, 2) pVER9662 (SEQ ID NO: 118) with the AT-UBI PRO driving expression of the Arabidopsis WUS gene, 3) UBIGMWUS (SEQ ID NO: 119) with the AT-UBI PRO driving expression of the Glycine max WUS gene, 4) UBIMTWUS (SEQ ID NO: 120) with the AT-UBI PRO driving expression of the Medicago truncatula WUS gene, 5) UBILJWUS (SEQ ID NO: 121) with the AT-UBI PRO driving expression of the Lotus japonica WUS gene, 6) UBIPVWUS (SEQ ID NO: 122) with the AT-UBI PRO driving expression of the Phaseolus vulgaris WUS gene, and 7) UBIPYWUS (SEQ ID NO: 123) with the AT-UBI PRO driving expression of the petunia WUS gene.

Various explant types, including immature cotyledons, split seeds, isolated embryonic axes, mature cotyledonary nodes, hypocotyl, epicotyl or leaf tissue of Brassica are isolated for transformation with the particle gun, co-introducing a mixture of two plasmids, the first containing an expression cassette consisting of the AT-UBI PRO driving expression of the cDNA sequence for each of the WUS orthologs (pVER9662 (SEQ ID NO: 118), UBIGMWUS (SEQ ID NO: 119), UBIMTWUS (SEQ ID NO: 120), UBILJWUS (SEQ ID NO: 121), UBIPVWUS (SEQ ID NO: 122), and UBIPYWUS (SEQ ID NO: 123)) plus an expression cassette for ZS-YELLOW (QC318 (SEQ ID NO: 117)). The explants are cultured for two weeks. At two weeks, the number of fluorescing globular somatic embryos are counted and tabulated for each treatment.

Two weeks after particle gun transformation, fluorescent globular somatic embryos are rarely observed on the bombarded control explant types, including immature cotyledons, split seeds, isolated embryonic axes, mature cotyledonary nodes, hypocotyl, epicotyl or leaf tissue of Brassica, while for all the other treatments (containing WUS genes from different dicot species driven by the AT-UBI promoter), numerous fluorescent somatic embryos are observed on bombarded explant types, including immature cotyledons, split seeds, isolated embryonic axes, mature cotyledonary nodes, hypocotyl, epicotyl or leaf tissue of Brassica.

### Example 12. Orthologs of the Arabidopsis WUS Gene Function in Sunflower T0 Stimulate Somatic Embryo Formation

Various explant types, including immature cotyledons, split seeds, isolated embryonic axes, mature cotyledonary nodes, hypocotyl, epicotyl or leaf tissue of sunflower are isolated for transformation with orthologs of the Arabidopsis WUS gene. The following treatments are compared. For the particle gun transformation treatments, all contain plasmid QC318 (SEQ ID NO: 117) with GM-EF1A PRO::GM-EF1A INTRON1::ZS-YELLOW::NOS TERM+GM-SAMS PRO::GM-SAMS INTRON1::GM-ALS::GM-ALS TERM. The treatments include 1) a control with no addition genes, 2) pVER9662 (SEQ ID NO: 118) with the AT-UBI PRO driving expression of the Arabidopsis WUS gene, 3) UBIGMWUS (SEQ ID NO: 119) with the AT-UBI PRO driving expression of the Glycine max WUS gene, 4) UBIMTWUS (SEQ ID NO: 120) with the AT-UBI PRO driving expression of the Medicago truncatula WUS gene, 5) UBILJWUS (SEQ ID NO: 121) with the AT-UBI PRO driving expression of the Lotus japonica WUS gene, 6) UBIPVWUS (SEQ ID NO: 122) with the AT-UBI PRO driving expression of the Phaseolus vulgaris WUS gene, and 7) UBIPYWUS (SEQ ID NO: 123) with the AT-UBI PRO driving expression of the petunia WUS gene.

Various explant types, including immature cotyledons, split seeds, isolated embryonic axes, mature cotyledonary nodes, hypocotyl, epicotyl or leaf tissue of sunflower are isolated for transformation with the particle gun, co-introducing a mixture of two plasmids, the first containing an expression cassette consisting of the AT-UBI PRO driving expression of the cDNA sequence for each of the WUS orthologs (pVER9662 (SEQ ID NO: 118), UBIGMWUS (SEQ ID NO: 119), UBIMTWUS (SEQ ID NO: 120), UBILJWUS (SEQ ID NO: 121), UBIPVWUS (SEQ ID NO: 122), and UBIPYWUS (SEQ ID NO: 123)) plus an expression cassette for ZS-YELLOW (QC318 (SEQ ID NO: 117)). The explants are cultured for two weeks. At two weeks, the number of fluorescing globular somatic embryos are counted and tabulated for each treatment.

Two weeks after particle gun transformation, fluorescent globular somatic embryos are rarely observed on the bombarded control explant types, including immature cotyledons, split seeds, isolated embryonic axes, mature cotyledonary nodes, hypocotyl, epicotyl or leaf tissue of sunflower, while for all the other treatments (containing WUS genes from different dicot species driven by the AT-UBI promoter), numerous fluorescent somatic embryos are observed on bombarded explant types, including immature cotyledons, split seeds, isolated embryonic axes, mature cotyledonary nodes, hypocotyl, epicotyl or leaf tissue of sunflower.

### Example 13. Orthologs of the Arabidopsis WUS Gene Stimulate Morphogenic Growth in Brassica

WUS genes from 12 different dicotyledonous species, 2 gymnosperms, and one monocot species were tested for efficacy by assessing their ability to stimulate growth of transgenic green shoot responses in Brassica while undergoing selection on spectinomycin-containing medium. For all treatments containing a WUS expression cassette, the configuration of the T-DNA was identical with the exception of the WUS gene used in the construct. This T-DNA configuration was RB+CAMV35S PRO::WUS::OS-T28 TERM+GM-UBQ PRO::GM-UBQ 5UTR::GM-UBQ INTRON1::ZS-YELLOW1 N1::NOS TERM+AT-UBIQ10 PRO::AT-UBIQ10 5UTR::AT-UBIQ10 INTRON1::CTP::SPCN::UBQ14 TERM+LB with the variable WUS gene in bold and italics.

Seeds of Brassica napus were surface sterilized in a 50% Clorox solution and germinated on solid medium containing MS basal salts and vitamins. The seedlings were grown at 28° C. in the light for 10 to 14 days, and the hypocotyls were dissected away from the cotyledons. The hypocotyl explants were transferred into 100×25 mm petri plates containing 10 mls of 20A medium (Table 3) with 200 mM acetosyringone and then sliced into sections 3-5 mm long. After slicing, 40 μl of Agrobacterium solution (Agrobacterium strain LBA4404 THY—at an Optical Density of 0.50 at 550 nM) containing the expression cassettes described above were added to the plates, and the petri plates containing the hypocotyl/Agrobacterium mixture were placed on a shaker platform and lightly agitated for 10 minutes. After 10 minutes of gentle agitation, the plates were moved into dim light and 21° C. for 3 days of co-cultivation.

After co-cultivation, the hypocotyl explants were removed from the Agrobacterium solution, and lightly blotted onto sterile filter paper before placing onto 70A selection media (Table 3) (containing 10 mg/l spectinomycin) and moved to the light room (26° C. and bright light). Explants remained on 70A selection media for two weeks prior to transfer to a second round of 70A selection (alternatively, explants were moved to 70B medium (Table 3) with 20 mg/l spectinomycin for the second round of selection). After two rounds of selection the explants were transferred to 70C shoot elongation media (Table 3) for 2-3 weeks and placed back into the light room. Shoots were then transferred onto 90A rooting media (Table 3) before being transferred to soil in the greenhouse.

TABLE 3
Media For Canola Transformation

Infection and co-cultivation medium (20A); MS salts and vitamins, 0.1 g/l myo-inositol,
0.5 g/l MES buffer, 0.1 mg/l a-NAA, 1 mg/l BAP, 10 ug/l gibberelic acid, 50 mg/l
thymidine, pH 5.5.
First Selection medium (70A); MS salts and vitamins, 0.1 g/l myo-inositol, 0.5 g/l
MES buffer, 0.1 mg/l a-NAA, 1 mg/l BAP, 10 ug/l gibberelic acid, 40 mg/l adenine
hemisulfate, 20 g/l sucrose, 0.5 g/l PVP40, 2 mg/l silver nitrate, 0.5 g/l carbenicillin,
5 mg/l spectinomycin, 5 g/l TC agar, pH 5.7.
Second Selection medium (70B); MS salts and vitamins, 0.1 g/l myo-inositol, 0.5 g/l
MES buffer, 0.1 mg/l a-NAA, 1 mg/l BAP, 10 ug/l gibberelic acid, 40 mg/l adenine
hemisulfate, 20 g/l sucrose, 0.5 g/l PVP40, 2 mg/l silver nitrate, 0.5 g/l carbenicillin,
10 mg/l spectinomycin, 5 g/l TC agar, pH 5.7.
Regeneration Medium (70C); MS salts and vitamins, 0.1 g/l myo-inositol, 0.5 g/l
MES buffer, 2.5 ug/l BAP, 40 mg/l adenine hemisulfate, 20 g/l sucrose, 0.5 g/l
PVP40, 0.5 g/l carbenicillin, 10 mg/l spectinomycin, 5 g/l TC agar, pH 5.7.
Rooting Medium (90A); MS salts and vitamins, 0.5 mg/l IBA, pH 5.7.

As shown in Table 4 WUS genes from different species stimulated growth responses of transgenic green shoots in canola. This stimulation of shoot development and the ability to recover spectinomycin-resistant shoots was variable, depending on the source of the WUS gene. For cucumber, this stimulation of transgenic shoots was marginally above the levels seen in the negative control treatments, but ranged for WUS genes from other species up to 95% for Gnetum gnemon (a gymnosperm), with a response above 70% being observed for WUS genes from grape, eelgrass (a monocot), Kalanchoe, petunia, apple, sunflower, cassava and Gnetum.

TABLE 4
Efficacy* of WUS genes from various species

| | | WUS | | | | | |
|---|---|---|---|---|---|---|---|
| | Plasmid | Gene | | | | # | |
| Plasmid | SEQ ID | SEQ ID | | | Total | green | % WUS |
| Number | NO: | NO: | Genus species | Common name | Explants | shoots | response |
| RV021090 | 153 | 78 | Helianthus annuus | Sunflower | 128 | 110 | 86% |
| RV026522 | 154 | 127 | Gnetum gnemon | Gnetum | 41 | 39 | 95% |
| RV026526 | 155 | 82 | Vitis vinifera | Grape | 81 | 58 | 72% |
| RV026534 | 156 | 90 | Petunia hybrida | Petunia | 121 | 97 | 80% |
| RV026531 | 157 | 129 | Malus domestica | Apple | 43 | 35 | 81% |
| RV026524 | 158 | 131 | Manihot esculenta | Cassava | 38 | 34 | 89% |
| RV026523 | 159 | 133 | Kalanchoe fedtschenkoi | Lavander scallops | 40 | 31 | 78% |
| RV026530 | 160 | 135 | Gossypium hirsutum | Cotton | 41 | 22 | 54% |
| RV026527 | 161 | 137 | Zostera marina | Eelgrass | 40 | 30 | 75% |
| RV026529 | 162 | 139 | Amborella trichopoda | Amborella | 44 | 20 | 45% |
| RV026528 | 163 | 141 | Aquilegia coerulea | Columbine | 40 | 21 | 53% |
| RV026520 | 164 | 80 | Populus trichocarpa | Poplar | 49 | 7 | 14% |
| RV026533 | 165 | 143 | Amaranthus hypochondriacus | Amaranth | 41 | 6 | 15% |
| RV026521 | 166 | 145 | Cucumis sativus | Cucumber | 69 | 4 | 6% |
| RV026525 | 167 | 147 | Pinus taeda | Pine | 43 | 5 | 12% |
| RV022814 | 168 | NA | None | No Wus | 38 | 2 | 5% |
| no agro | NA | NA | None | no agro | 124 | 2 | 2% |

*Efficacy of WUS genes from various species, as measured by the percentage of starting hypocotyl explants that produced green, fluorescent shoots from the explants (on spectinomycin selection) at 28 days after Agrobacterium infection, wherein the starting hypocotyl explants were transformed by an Agrobacterium with a T-DNA containing a WUS gene as described above.

### Example 14. Use of Promoters from the Hd-Zip IV Transcription Factor Family Controlling Expression of a Sunflower WUS Ortholog Stimulate Morphogenic Growth in Brassica

Arabidopsis promoters driving expression of an ortholog of the Arabidopsis WUS gene from sunflower (Helianthus annuus) were tested for efficacy by assessing their ability to stimulate growth of transgenic green shoot responses in Brassica while undergoing selection on spectinomycin-containing medium. For all treatments a T-DNA was used containing three expression cassettes RB+HD-ZIP IV PRO::HA-WUS::OS-T28 TERM+GM-UBQ PRO::GM-UBQ 5UTR::GM-UBQ INTRON1::ZS-YELLOW1 N1::NOS TERM+AT-UBIQ10 PRO::AT-UBIQ10 5UTR::AT-UBIQ10 INTRON1::CTP::SPCN::UBQ14 TERM+LB, with the variable promoter HD-ZIP IV PRO in bold and italics.

The promoters tested were from Arabidopsis, and included Protodermal Factor 1 (PDF1, SEQ ID NO: 149), Protodermal Factor2 (PDF2, SEQ ID NO: 150), Glabrous2 (HDG2, SEQ ID NO: 151), Meristem Layer1 (ML1, SEQ ID NO: 125), Eceriferum6 (CER6, SEQ ID NO: 126), and Anthocyanless (ANL1, SEQ ID NO: 152). The T-DNA configuration for the plasmids containing these promoters is shown in Table 1 in RV027343, RV027344, RV027340, RV027342, RV027338, and RV027337, respectively, which correspond to SEQ ID NOS. 175, 169, 173, 174, 172, and 171, respectively. These promoters were compared to the CaMV35S PRO (for T-DNA information, see RV021090, SEQ ID NO: 153) as a positive control, and a T-DNA with no WUS expression cassette as a negative control (for T-DNA information, see RV026532, SEQ ID NO: 170).

Surface sterilization of Brassica seed, germination to produce seedlings, preparation of the hypocotyl explants, transformation with Agrobacterium strain LBA4404 THY-, tissue culture and selection using spectinomycin were all performed as described in Example 13.

Results are presented on a scale of 0 to 10, with zero (0) being no response beyond that of the negative control and ten (10) matching the strong morphogenic response stimulation observed with the CaMV35S PRO. For the various promoters tested, the response observed with the PDF1 PRO was 5, for the PDF2 PRO the response was 2.3, for the HDG2 PRO the response was 2.2, for the ML1 the response was 2, for the CER6 PRO the response was 2, and for the ANL1 PRO the response was 2. The CaMV35S promoter is a strong constitutive promoter which can be undesirable when driving expression of WUS; while strong initial expression is beneficial immediately after integration of the T-DNA (to rapidly stimulate shoot formation), strong expression in the whole plant results in morphological abnormalities and sterility. The HD-ZIP IV promoters tested were expressed in the L1 (epidermal) layer of developing embryos and meristems, and are otherwise not expressed in the whole plant. Thus, a positive growth stimulation of shoot formation after T-DNA delivery with down-regulation of the WUS expression as the vegetative and reproductive portions of the plant develop was the desired result and was achieved. This rapid growth stimulation of shoot formation was demonstrated for all the HD-ZIP IV promoters tested.

1. A nucleic acid molecule comprising a morphogenic gene cassette comprising a tissue-preferred promoter having a nucleotide sequence selected from the group consisting of:
(a) at least one of SEQ ID NOS: 1-59, 108-110, 124-126, 149-152, and 189, wherein the nucleotide sequence initiates transcription in a plant cell;
(b) a nucleotide sequence that is at least 95% identical to at least one of SEQ ID NOS: 1-59, 108-110, 124-126, 149-152, and 189, wherein the nucleotide sequence initiates transcription in a plant cell;
(c) a nucleotide sequence that is at least 70% identity to at least one of SEQ ID NOS: 1-59, 108-110, 124-126, 149-152, and 189, wherein the nucleotide sequence initiates transcription in a plant cell;
(d) a fragment or variant of the nucleotide sequence of at least one of SEQ ID NOS: 1-59, 108-110, 124-126, 149-152, and 189, wherein the fragment or variant of the nucleotide sequence initiates transcription in a plant cell; or
(e) at least 100 contiguous nucleotides of a nucleotide sequence of at least one of SEQ ID NOS: 1-59, 108-110, 124-126, 149-152, and 189, wherein the at least 100 contiguous nucleotides of the nucleotide sequence initiates transcription in a plant cell;
wherein the tissue-preferred promoter of (a)-(e) is operably linked to a morphogenic gene, wherein the morphogenic gene encodes a WUS/WOX homeobox polypeptide,
wherein the WUS/WOX homeobox polypeptide comprises an amino acid sequence of any of SEQ ID NOS: 61, 63, 65, 67, 69, 71, 73, 75, 77, 79, 81, 83, 85, 87, 89, 91, 93, 128, 130, 132, 134, 136, 138, 140, 142, 144, 146, or 148; or wherein the WUS/WOX homeobox polypeptide is encoded by a nucleotide sequence of any of SEQ ID NOS: 60, 62, 64, 66, 68, 70, 72, 74, 76, 78, 80, 82, 84, 86, 88, 90, 92, 127, 129, 131, 133, 135, 137, 139, 141, 143, 145, or 147.

2. An expression cassette comprising the morphogenic gene cassette of claim 1.

3. A vector comprising the expression cassette of claim 2.

4. A plant cell comprising the expression cassette of claim 2.

5. The plant cell of claim 4, wherein the plant cell is from a monocot, a dicot, or a gymnosperm.

6. The plant cell of claim 5, wherein the monocot, the dicot, or the gymnosperm is selected from the group consisting of maize, alfalfa, sorghum, rice, millet, soybean, wheat, cotton, sunflower, barley, oats, rye, flax, sugarcane, banana, cassava, common bean, cowpea, tomato, potato, beet, grape, Eucalyptus, poplar, pine, douglas fir, citrus, papaya, cacao, cucumber, apple, Capsicum, bamboo, Triticale, melon, or Brassica.

7-8. (canceled)

9. The plant cell of claim 4, wherein the expression cassette further comprises a trait gene cassette comprising a heterologous polynucleotide encoding a gene product conferring nutritional enhancement, increased yield, abiotic stress tolerance, drought tolerance, cold tolerance, herbicide tolerance, pest resistance, pathogen resistance, insect resistance, nitrogen use efficiency (NUE), disease resistance, or an ability to alter a metabolic pathway.

10. The plant cell of claim 9, wherein the expression cassette further comprises a site-specific recombinase cassette comprising a nucleotide sequence encoding a site-specific recombinase selected from the group consisting of FLP, FLPe, KD, Cre, SSV1, lambda Int, phi C31 Int, HK022, R, B2, B3, Gin, Tn1721, CinH, ParA, Tn5053, Bxb1, TP907-1, or U153, wherein the site-specific recombinase is operably linked to a constitutive promoter, an inducible promoter, a tissue-specific promoter, or a developmentally regulated promoter.

11. The plant cell of claim 10, wherein the constitutive promoter, the inducible promoter, the tissue-specific promoter, or the developmentally regulated promoter is selected from the group consisting of UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, ZM-ADF PRO (ALT2), AXIG1, DR5, XVE, GLB1, OLE, LTP2, HSP17.7, HSP26, HSP18A, AT-HSP811, AT-HSP811L, GM-HSP173B, promoters activated by tetracycline, ethamethsulfuron or chlorsulfuron, PLTP, PLTP1, PLTP2, PLTP3, SDR, LGL, LEA-14A, or LEA-D34.

12. The plant cell of claim 11, wherein the morphogenic gene cassette and the site-specific recombinase cassette of the expression cassette are transiently expressed in the plant cell and the trait gene cassette of the expression cassette is stably incorporated into the genome of the plant cell.

13. The plant cell of claim 11, wherein the morphogenic gene cassette and the site-specific recombinase cassette of the expression cassette are excised from the plant cell and the trait gene cassette of the expression cassette is stably incorporated into the genome of the plant cell.

14. A plant comprising the expression cassette of claim 2.

15. The plant of claim 14, wherein the plant is a monocot, a dicot, or a gymnosperm.

16. The plant of claim 15, wherein the monocot, the dicot, or the gymnosperm is selected from the group consisting of maize, alfalfa, sorghum, rice, millet, soybean, wheat, cotton, sunflower, barley, oats, rye, flax, sugarcane, banana, cassava, common bean, cowpea, tomato, potato, beet, grape, Eucalyptus, poplar, pine, douglas fir, citrus, papaya, cacao, cucumber, apple, Capsicum, bamboo, Triticale, melon, or Brassica.

17-18. (canceled)

19. The plant of claim 14, wherein the expression cassette further comprises a trait gene cassette comprising a heterologous polynucleotide encoding a gene product conferring nutritional enhancement, increased yield, abiotic stress tolerance, drought tolerance, cold tolerance, herbicide tolerance, pest resistance, pathogen resistance, insect resistance, nitrogen use efficiency (NUE), disease resistance, or an ability to alter a metabolic pathway.

20. The plant of claim 19, wherein the expression cassette further comprises a site-specific recombinase cassette comprising a nucleotide sequence encoding a site-specific recombinase selected from the group consisting of FLP, FLPe, KD, Cre, SSV1, lambda Int, phi C31 Int, HK022, R, B2, B3, Gin, Tn1721, CinH, ParA, Tn5053, Bxb1, TP907-1, or U153, wherein the site-specific recombinase is operably linked to a constitutive promoter, an inducible promoter, a tissue-specific promoter, or a developmentally regulated promoter.

21. The plant of claim 20, wherein, wherein the constitutive promoter, the inducible promoter, the tissue-specific promoter, or the developmentally regulated promoter is selected from the group consisting of UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, ZM-ADF PRO (ALT2), AXIG1, DR5, XVE, GLB1, OLE, LTP2, HSP17.7, HSP26, HSP18A, AT-HSP811, AT-HSP811L, GM-HSP173B, promoters activated by tetracycline, ethamethsulfuron or chlorsulfuron, PLTP, PLTP1, PLTP2, PLTP3, SDR, LGL, LEA-14A, or LEA-D34.

22. The plant of claim 21, wherein the morphogenic gene cassette and the site-specific recombinase cassette of the expression cassette are transiently expressed in the plant and the trait gene cassette of the expression cassette is stably incorporated into the genome of the plant.

23. A seed of the plant of claim 22, wherein the seed comprises the trait gene cassette of the expression cassette.

24. The plant of claim 21, wherein the morphogenic gene cassette and the site-specific recombinase cassette of the expression cassette are excised from the plant and the trait gene cassette of the expression cassette is stably incorporated into the genome of the plant.

25. A seed of the plant of claim 24, wherein the seed comprises the trait gene cassette of the expression cassette.

26-30. (canceled)

31. A method of producing a transgenic plant comprising:
transforming a plant cell with a recombinant expression cassette comprising (a) a tissue-preferred promoter cassette, wherein the tissue-preferred promoter cassette comprises a nucleotide sequence selected from the group consisting of:
(i) at least one of SEQ ID NOS: 1-59, 108-110, 124-126, 149-152, and 189, wherein the nucleotide sequence initiates transcription in the plant cell;
(ii) a nucleotide sequence that is at least 95% identical to at least one of SEQ ID NOS: 1-59, 108-110, 124-126, 149-152, and 189, wherein the nucleotide sequence initiates transcription in the plant cell;
(iii) a nucleotide sequence that is at least 70% identical to at least one of SEQ ID NOS: 1-59, 108-110, 124-126, 149-152, and 189, wherein the nucleotide sequence initiates transcription in the plant cell;
(iv) a nucleotide sequence that is a fragment or variant of at least one of SEQ ID NOS: 1-59, 108-110, 124-126, 149-152, and 189, wherein the nucleotide sequence initiates transcription in the plant cell; or
(v) a nucleotide sequence that is at least a 100-bp fragment of at least one of SEQ ID NOS: 1-59, 108-110, 124-126, 149-152, and 189, wherein the nucleotide sequence initiates transcription in the plant cell, wherein the nucleotide sequence of (i)-(v) is operably linked to a morphogenic gene, wherein the morphogenic gene encodes a WUS/WOX homeobox polypeptide,
wherein the WUS/WOX homeobox polypeptide comprises an amino acid sequence of any of SEQ ID NOS: 61, 63, 65, 67, 69, 71, 73, 75, 77, 79, 81, 83, 85, 87, 89, 91, 93, 128, 130, 132, 134, 136, 138, 140, 142, 144, 146, or 148; or wherein the WUS/WOX homeobox polypeptide is encoded by a nucleotide sequence of any of SEQ ID NOS: 60, 62, 64, 66, 68, 70, 72, 74, 76, 78, 80, 82, 84, 86, 88, 90, 92, 127, 129, 131, 133, 135, 137, 139, 141, 143, 145, or 147, and (b) a trait gene cassette comprising a heterologous polynucleotide of interest encoding a gene product conferring nutritional enhancement, increased yield, abiotic stress tolerance, drought tolerance, cold tolerance, herbicide tolerance, pest resistance, pathogen resistance, insect resistance, nitrogen use efficiency (NUE), disease resistance, or an ability to alter a metabolic pathway;
selecting a transgenic plant cell having the recombinant expression cassette; and
regenerating the transgenic plant from the transgenic plant cell.

32. The method of claim 31, wherein the plant cell is a monocot, a dicot, or gymnosperm.

33. The method of claim 32, wherein the monocot, the dicot, or the gymnosperm is selected from the group consisting of maize, alfalfa, sorghum, rice, millet, soybean, wheat, cotton, sunflower, barley, oats, rye, flax, sugarcane, banana, cassava, common bean, cowpea, tomato, potato, beet, grape, Eucalyptus, poplar, pine, douglas fir, citrus, papaya, cacao, cucumber, apple, Capsicum, bamboo, Triticale, melon, or Brassica.

34-35. (canceled)

36. The method of claim 31, wherein the expression cassette further comprises a site-specific recombinase cassette comprising a nucleotide sequence encoding a site-specific recombinase selected from the group consisting of FLP, FLPe, KD, Cre, SSV1, lambda Int, phi C31 Int, HK022, R, B2, B3, Gin, Tn1721, CinH, ParA, Tn5053, Bxb1, TP907-1, or U153, wherein the site-specific recombinase is operably linked to a constitutive promoter, an inducible promoter, a tissue-specific promoter, or a developmentally regulated promoter.

37. The method of claim 36, wherein, wherein the constitutive promoter, the inducible promoter, the tissue-specific promoter, or the developmentally regulated promoter is selected from the group consisting of UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, ZM-ADF PRO (ALT2), AXIG1, DR5, XVE, GLB1, OLE, LTP2, HSP17.7, HSP26, HSP18A, AT-HSP811, AT-HSP811L, GM-HSP173B, promoters activated by tetracycline, ethamethsulfuron or chlorsulfuron, PLTP, PLTP1, PLTP2, PLTP3, SDR, LGL, LEA-14A, or LEA-D34.

38. The method of claim 37, further comprising excising the tissue-preferred promoter cassette and the site-specific recombinase cassette from the recombinant expression cassette.

39. The transgenic plant produced by the method of claim 38.

40. A seed of the transgenic plant of claim 39, wherein the seed comprises the trait gene cassette of the recombinant expression cassette.

41. The method of claim 31, wherein the tissue-preferred promoter cassette comprises a first T-DNA and the trait gene cassette comprises a second T-DNA.

42. The method of claim 41, wherein the first T-DNA and the second T-DNA reside in the same bacterial strain for transforming the plant cell.

43. The method of claim 42, further comprising segregating the first T-DNA away from the second T-DNA.

44. The transgenic plant produced by the method of claim 43.

45. A seed of the transgenic plant of claim 44, wherein the seed comprises the trait gene cassette of the recombinant expression cassette.

46. The method of claim 41, wherein the first T-DNA resides in a first bacterial strain and the second T-DNA resides in a second bacterial strain and the first bacterial strain and the second bacterial strain are mixed in a ratio for transforming the plant cell.

47. The method of claim 46, further comprising segregating the first T-DNA away from the second T-DNA.

48. The transgenic plant produced by the method of claim 47.

49. A seed of the transgenic plant of claim 48, wherein the seed comprises the trait gene cassette of the recombinant expression cassette.

50. A method of improving somatic embryo maturation efficiency or regeneration efficiency comprising:
transforming a plant cell with a recombinant expression cassette comprising (a) a tissue-preferred promoter cassette, wherein the tissue-preferred promoter cassette comprises a nucleotide sequence selected from the group consisting of:
(i) at least one of SEQ ID NOS: 1-59, 108-110, 124-126, 149-152, and 189, wherein the nucleotide sequence initiates transcription in the plant cell;
(ii) a nucleotide sequence that is at least 95% identical to at least one of SEQ ID NOS: 1-59, 108-110, 124-126, 149-152, and 189, wherein the nucleotide sequence initiates transcription in the plant cell;
(iii) a nucleotide sequence that is at least 70% identical to at least one of SEQ ID NOS: 1-59, 108-110, 124-126, 149-152, and 189, wherein the nucleotide sequence initiates transcription in the plant cell;
(iv) a nucleotide sequence that is a fragment or variant of at least one of SEQ ID NOS: 1-59, 108-110, 124-126, 149-152, and 189, wherein the nucleotide sequence initiates transcription in the plant cell;
(v) a nucleotide sequence that is at least a 100-bp fragment of at least one of SEQ ID NOS: 1-59, 108-110, 124-126, 149-152, and 189, wherein the nucleotide sequence initiates transcription in the plant cell, wherein the nucleotide sequence of (a)-(e) is operably linked to a morphogenic gene, wherein the morphogenic gene encodes a WUS/WOX homeobox polypeptide,
wherein the WUS/WOX homeobox polypeptide comprises an amino acid sequence of any of SEQ ID NOS: 61, 63, 65, 67, 69, 71, 73, 75, 77, 79, 81, 83, 85, 87, 89, 91, 93, 128, 130, 132, 134, 136, 138, 140, 142, 144, 146, or 148; or wherein the WUS/WOX homeobox polypeptide is encoded by a nucleotide sequence of any of SEQ ID NOS: 60, 62, 64, 66, 68, 70, 72, 74, 76, 78, 80, 82, 84, 86, 88, 90, 92, 127, 129, 131, 133, 135, 137, 139, 141, 143, 145, or 147, and (b) a trait gene cassette comprising a heterologous polynucleotide of interest encoding a gene product conferring nutritional enhancement, increased yield, abiotic stress tolerance, drought tolerance, cold tolerance, herbicide tolerance, pest resistance, pathogen resistance, insect resistance, nitrogen use efficiency (NUE), disease resistance, or an ability to alter a metabolic pathway;
selecting a transgenic plant cell having the recombinant expression cassette; and
regenerating the transgenic plant from the transgenic plant cell, wherein the recombinant expression cassette results in improved somatic embryo maturation efficiency or regeneration efficiency when compared to a transgenic plant cell not comprising the recombinant expression cassette.

51. The method of claim 50, wherein the plant cell is a monocot, a dicot, or a gymnosperm.

52. The method of claim 51, wherein the monocot, the dicot, or the gymnosperm is selected from the group consisting of maize, alfalfa, sorghum, rice, millet, soybean, wheat, cotton, sunflower, barley, oats, rye, flax, sugarcane, banana, cassava, common bean, cowpea, tomato, potato, beet, grape, Eucalyptus, poplar, pine, douglas fir, citrus, papaya, cacao, cucumber, apple, Capsicum, bamboo, Triticale, melon, or Brassica.

53-54. (canceled)

55. The method of claim 50, wherein the expression cassette further comprises a site-specific recombinase cassette comprising a nucleotide sequence encoding a site-specific recombinase selected from the group consisting of FLP, FLPe, KD, Cre, SSV1, lambda Int, phi C31 Int, HK022, R, B2, B3, Gin, Tn1721, CinH, ParA, Tn5053, Bxb1, TP907-1, or U153, wherein the site-specific recombinase is operably linked to a constitutive promoter, an inducible promoter, a tissue-specific promoter, or a developmentally regulated promoter.

56. The method of claim 55, wherein, wherein the constitutive promoter, the inducible promoter, the tissue-specific promoter, or the developmentally regulated promoter is selected from the group consisting of UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, ZM-ADF PRO (ALT2), AXIG1, DR5, XVE, GLB1, OLE, LTP2, HSP17.7, HSP26, HSP18A, AT-HSP811, AT-HSP811L, GM-HSP173B, promoters activated by tetracycline, ethamethsulfuron or chlorsulfuron, PLTP, PLTP1, PLTP2, PLTP3, SDR, LGL, LEA-14A, or LEA-D34.

57. The method of claim 56, further comprising excising the tissue-preferred promoter cassette and the site-specific recombinase cassette from the recombinant expression cassette.

58. The transgenic plant produced by the method of claim 57.

59. A seed of the transgenic plant of claim 58, wherein the seed comprises the trait gene cassette of the recombinant expression cassette.

60. The method of claim 50, wherein the tissue-preferred promoter cassette comprises a first T-DNA and the trait gene cassette comprises a second T-DNA.

61. The method of claim 60, wherein the first T-DNA and the second T-DNA reside in the same bacterial strain for transforming the plant cell.

62. The method of claim 61, further comprising segregating the first T-DNA away from the second T-DNA.

63. The transgenic plant produced by the method of claim 62.

64. A seed of the transgenic plant of claim 63, wherein the seed comprises the trait gene cassette of the recombinant expression cassette.

65. The method of claim 60, wherein the first T-DNA resides in a first bacterial strain and the second T-DNA resides in a second bacterial strain and the first bacterial strain and the second bacterial strain are mixed in a ratio for transforming the plant cell.

66. The method of claim 65, further comprising segregating the first T-DNA away from the second T-DNA.

67. The transgenic plant produced by the method of claim 66.

68. A seed of the transgenic plant of claim 67, wherein the seed comprises the trait gene cassette of the recombinant expression cassette.

69-111. (canceled)
